SEQUENCE LISTING (129 sequences)

SEQ ID NO: 1 - DNA, 990 bp, Escherichia coli
  source 1..990: mol_type = genomic DNA
atgaaactcgccgtttatagcacaaaacagtacgacaagaagtacctgcaacaggtgaacgagtcctttggctttgagctggaattttttgactttctgctgacggaaaaaaccgctaaaactgccaatggctgcgaagcggtatgtattttcgtaaacgatgacggcagccgcccggtgctggaagagctgaaaaagcacggcgttaaatatatcgccctgcgctgtgccggtttcaataacgtcgaccttgacgcggcaaaagaactggggctgaaagtagtccgtgttccagcctatgatccagaggccgttgctgaacacgccatcggtatgatgatgacgctgaaccgccgtattcaccgcgcgtatcagcgtacccgtgatgctaacttctctctggaaggtctgaccggctttactatgtatggcaaaacggcaggcgttatcggtaccggtaaaatcggtgtggcgatgctgcgcattctgaaaggttttggtatgcgtctgctggcgttcgatccgtatccaagtgcagcggcgctggaactcggtgtggagtatgtcgatctgccaaccctgttctctgaatcagacgttatctctctgcactgcccgctgacaccggaaaactatcatctgttgaacgaagccgccttcgaacagatgaaaaatggcgtgatgatcgtcaataccagtcgcggtgcattgattgattctcaggcagcaattgaagcgctgaaaaatcagaaaattggttcgttgggtatggacgtgtatgagaacgaacgcgatctattctttgaagataaatccaacgacgtgatccaggatgacgtattccgtcgcctgtctgcctgccacaacgtgctgtttaccgggcaccaggcattcctgacagcagaagctctgaccagtatttctcagactacgctgcaaaacttaagcaatctggaaaaaggcgaaacctgcccgaacgaactggtttaa

SEQ ID NO: 2 - protein, 329 aa, Escherichia coli
MKLAVYSTKQYDKKYLQQVNESFGFELEFFDFLLTEKTAKTANGCEAVCIFVNDDGSRPVLEELKKHGVKYIALRCAGFNNVDLDAAKELGLKVVRVPAYDPEAVAEHAIGMMMTLNRRIHRAYQRTRDANFSLEGLTGFTMYGKTAGVIGTGKIGVAMLRILKGFGMRLLAFDPYPSAAALELGVEYVDLPTLFSESDVISLHCPLTPENYHLLNEAAFEQMKNGVMIVNTSRGALIDSQAAIEALKNQKIGSLGMDVYENERDLFFEDKSNDVIQDDVFRRLSACHNVLFTGHQAFLTAEALTSISQTTLQNLSNLEKGETCPNELV

SEQ ID NO: 3 - DNA, 2676 bp, Escherichia coli
  source 1..2676: mol_type = genomic DNA
atggctgttactaatgtcgctgaacttaacgcactcgtagagcgtgtaaaaaaagcccagcgtgaatatgccagtttcactcaagagcaagtagacaaaatcttccgcgccgccgctctggctgctgcagatgctcgaatcccactcgcgaaaatggccgttgccgaatccggcatgggtatcgtcgaagataaagtgatcaaaaaccactttgcttctgaatatatctacaacgcctataaagatgaaaaaacctgtggtgttctgtctgaagacgacacttttggtaccatcactatcgctgaaccaatcggtattatttgcggtatcgttccgaccactaacccgacttcaactgctatcttcaaatcgctgatcagtctgaagacccgtaacgccattatcttctccccgcacccgcgtgcaaaagatgccaccaacaaagcggctgatatcgttctgcaggctgctatcgctgccggtgctccgaaagatctgatcggctggatcgatcaaccttctgttgaactgtctaacgcactgatgcaccacccagacatcaacctgatcctcgcgactggtggtccgggcatggttaaagccgcatacagctccggtaaaccagctatcggtgtaggcgcgggcaacactccagttgttatcgatgaaactgctgatatcaaacgtgcagttgcatctgtactgatgtccaaaaccttcgacaacggcgtaatctgtgcttctgaacagtctgttgttgttgttgactctgtttatgacgctgtacgtgaacgttttgcaacccacggcggctatctgttgcagggtaaagagctgaaagctgttcaggatgttatcctgaaaaacggtgcgctgaacgcggctatcgttggtcagccagcctataaaattgctgaactggcaggcttctctgtaccagaaaacaccaagattctgatcggtgaagtgaccgttgttgatgaaagcgaaccgttcgcacatgaaaaactgtccccgactctggcaatgtaccgcgctaaagatttcgaagacgcggtagaaaaagcagagaaactggttgctatgggcggtatcggtcatacctcttgcctgtacactgaccaggataaccaaccggctcgcgtttcttacttcggtcagaaaatgaaaacggcgcgtatcctgattaacaccccagcgtctcagggtggtatcggtgacctgtataacttcaaactcgcaccttccctgactctgggttgtggttcttggggtggtaactccatctctgaaaacgttggtccgaaacacctgatcaacaagaaaaccgttgctaagcgagctgaaaacatgttgtggcacaaacttccgaaatctatctacttccgccgtggctccctgccaatcgcgctggatgaagtgattactgatggccacaaacgtgcgctcatcgtgactgaccgcttcctgttcaacaatggttatgctgatcagatcacttccgtactgaaagcagcaggcgttgaaactgaagtcttcttcgaagtagaagcggacccgaccctgagcatcgttcgtaaaggtgcagaactggcaaactccttcaaaccagacgtgattatcgcgctgggtggtggttccccgatggacgccgcgaagatcatgtgggttatgtacgaacatccggaaactcacttcgaagagctggcgctgcgctttatggatatccgtaaacgtatctacaagttcccgaaaatgggcgtgaaagcgaaaatgatcgctgtcaccaccacttctggtacaggttctgaagtcactccgtttgcggttgtaactgacgacgctactggtcagaaatatccgctggcagactatgcgctgactccggatatggcgattgtcgacgccaacctggttatggacatgccgaagtccctgtgtgctttcggtggtctggacgcagtaactcacgccatggaagcttatgtttctgtactggcatctgagttctctgatggtcaggctctgcaggcactgaaactgctgaaagaatatctgccagcgtcctaccacgaagggtctaaaaatccggtagcgcgtgaacgtgttcacagtgcagcgactatcgcgggtatcgcgtttgcgaacgccttcctgggtgtatgtcactcaatggcgcacaaactgggttcccagttccatattccgcacggtctggcaaacgccctgctgatttgtaacgttattcgctacaatgcgaacgacaacccgaccaagcagactgcattcagccagtatgaccgtccgcaggctcgccgtcgttatgctgaaattgccgaccacttgggtctgagcgcaccgggcgaccgtactgctgctaagatcgagaaactgctggcatggctggaaacgctgaaagctgaactgggtattccgaaatctatccgtgaagctggcgttcaggaagcagacttcctggcgaacgtggataaactgtctgaagatgcattcgatgaccagtgcaccggcgctaacccgcgttacccgctgatctccgagctgaaacagattctgctggatacctactacggtcgtgattatgtagaaggtgaaactgcagcgaagaaagaagctgctccggctaaagctgagaaaaaagcgaaaaaatccgcttaa

SEQ ID NO: 4 - protein, 891 aa, Escherichia coli
MAVTNVAELNALVERVKKAQREYASFTQEQVDKIFRAAALAAADARIPLAKMAVAESGMGIVEDKVIKNHFASEYIYNAYKDEKTCGVLSEDDTFGTITIAEPIGIICGIVPTTNPTSTAIFKSLISLKTRNAIIFSPHPRAKDATNKAADIVLQAAIAAGAPKDLIGWIDQPSVELSNALMHHPDINLILATGGPGMVKAAYSSGKPAIGVGAGNTPVVIDETADIKRAVASVLMSKTFDNGVICASEQSVVVVDSVYDAVRERFATHGGYLLQGKELKAVQDVILKNGALNAAIVGQPAYKIAELAGFSVPENTKILIGEVTVVDESEPFAHEKLSPTLAMYRAKDFEDAVEKAEKLVAMGGIGHTSCLYTDQDNQPARVSYFGQKMKTARILINTPASQGGIGDLYNFKLAPSLTLGCGSWGGNSISENVGPKHLINKKTVAKRAENMLWHKLPKSIYFRRGSLPIALDEVITDGHKRALIVTDRFLFNNGYADQITSVLKAAGVETEVFFEVEADPTLSIVRKGAELANSFKPDVIIALGGGSPMDAAKIMWVMYEHPETHFEELALRFMDIRKRIYKFPKMGVKAKMIAVTTTSGTGSEVTPFAVVTDDATGQKYPLADYALTPDMAIVDANLVMDMPKSLCAFGGLDAVTHAMEAYVSVLASEFSDGQALQALKLLKEYLPASYHEGSKNPVARERVHSAATIAGIAFANAFLGVCHSMAHKLGSQFHIPHGLANALLICNVIRYNANDNPTKQTAFSQYDRPQARRRYAEIADHLGLSAPGDRTAAKIEKLLAWLETLKAELGIPKSIREAGVQEADFLANVDKLSEDAFDDQCTGANPRYPLISELKQILLDTYYGRDYVEGETAAKKEAAPAKAEKKAKKSA

SEQ ID NO: 5 - DNA, 459 bp, Escherichia coli
  source 1..459: mol_type = genomic DNA
atggaactgacgactcgcactttacctgcgcggaaacatattgcgctggtggcacacgatcactgcaaacaaatgctgatgagctgggtggaacggcatcaaccgttactggaacaacacgtactgtatgcaacaggcactaccggtaacttaatttcccgcgcgaccggcatgaacgtcaacgcgatgttgagtggcccaatggggggtgaccagcaggttggcgcattgatctcagaagggaaaattgatgtattgattttcttctgggatccactaaatgccgtgccgcacgatcctgacgtgaaagccttgctgcgtctggcgacggtatggaacattccggtcgccaccaacgtggcaacggcagacttcataatccagtcgccgcatttcaacgacgcggtcgatattctgatccccgattatcagcgttatctcgcggaccgtctgaagtaa

SEQ ID NO: 6 - protein, 152 aa, Escherichia coli [sequence omitted]

SEQ ID NO: 7 - DNA, 1689 bp, Escherichia coli
  source 1..1689: mol_type = genomic DNA
atggcaagttcgggcacaacatcgacgcgtaagcgctttaccggcgcagaatttatcgttcatttcctggaacagcagggcattaagattgtgacaggcattccgggcggttctatcctgcctgtttacgatgccttaagccaaagcacgcaaatccgccatattctggcccgtcatgaacagggcgcgggctttatcgctcagggaatggcgcgcaccgacggtaaaccggcggtctgtatggcctgtagcggaccgggtgcgactaacctggtgaccgccattgccgatgcgcggctggactccatcccgctgatttgcatcactggtcaggttcccgcctcgatgatcggcaccgacgccttccaggaagtggacacctacggcatctctatccccatcaccaaacacaactatctggtcagacatatcgaagaactcccgcaggtcatgagcgatgccttccgcattgcgcaatcaggccgcccaggcccggtgtggatagacattcctaaggatgtgcaaacggcagtttttgagattgaaacacagcccgctatggcagaaaaagccgccgcccccgcctttagcgaagaaagcattcgtgacgcagcggcgatgattaacgctgccaaacgcccggtgctttatctgggcggcggtgtgatcaatgcgcccgcacgggtgcgtgaactggcggagaaagcgcaactgcctaccaccatgactttaatggcgctgggcatgttgccaaaagcgcatccgttgtcgctgggtatgctggggatgcacggcgtgcgcagcaccaactatattttgcaggaggcggatttgttgatagtgctcggtgcgcgttttgatgaccgggcgattggcaaaaccgagcagttctgtccgaatgccaaaatcattcatgtcgatatcgaccgtgcagagctgggtaaaatcaagcagccgcacgtggcgattcaggcggatgttgatgacgtgctggcgcagttgatcccgctggtggaagcgcaaccgcgtgcagagtggcaccagttggtagcggatttgcagcgtgagtttccgtgtccaatcccgaaagcgtgcgatccgttaagccattacggcctgatcaacgccgttgccgcctgtgtcgatgacaatgcaattatcaccaccgacgttggtcagcatcagatgtggaccgcgcaagcttatccgctcaatcgcccacgccagtggctgacctccggtgggctgggcacgatgggttttggcctgcctgcggcgattggcgctgcgctggcgaacccggatcgcaaagtgttgtgtttctccggcgacggcagcctgatgatgaatattcaggagatggcgaccgccagtgaaaatcagctggatgtcaaaatcattctgatgaacaacgaagcgctggggctggtgcatcagcaacagagtctgttctacgagcaaggcgtttttgccgccacctatccgggcaaaatcaactttatgcagattgccgccggattcggcctcgaaacctgtgatttgaataacgaagccgatccgcaggcttcattgcaggaaatcatcaatcgccctggcccggcgctgatccatgtgcgcattgatgccgaagaaaaagtttacccgatggtgccgccaggtgcggcgaatactgaaatggtgggggaataa

SEQ ID NO: 8 - protein, 562 aa, Escherichia coli
MASSGTTSTRKRFTGAEFIVHFLEQQGIKIVTGIPGGSILPVYDALSQSTQIRHILARHEQGAGFIAQGMARTDGKPAVCMACSGPGATNLVTAIADARLDSIPLICITGQVPASMIGTDAFQEVDTYGISIPITKHNYLVRHIEELPQVMSDAFRIAQSGRPGPVWIDIPKDVQTAVFEIETQPAMAEKAAAPAFSEESIRDAAAMINAAKRPVLYLGGGVINAPARVRELAEKAQLPTTMTLMALGMLPKAHPLSLGMLGMHGVRSTNYILQEADLLIVLGARFDDRAIGKTEQFCPNAKIIHVDIDRAELGKIKQPHVAIQADVDDVLAQLIPLVEAQPRAEWHQLVADLQREFPCPIPKACDPLSHYGLINAVAACVDDNAIITTDVGQHQMWTAQAYPLNRPRQWLTSGGLGTMGFGLPAAIGAALANPDRKVLCFSGDGSLMMNIQEMATASENQLDVKIILMNNEALGLVHQQQSLFYEQGVFAATYPGKINFMQIAAGFGLETCDLNNEADPQASLQEIINRPGPALIHVRIDAEEKVYPMVPPGAANTEMVGE

SEQ ID NO: 9 - DNA, 291 bp, Escherichia coli
  source 1..291: mol_type = genomic DNA
atgcaaaacacaactcatgacaacgtaattctggagctcaccgttcgcaaccatccgggcgtaatgacccacgtttgtggcctttttgcccgccgcgcttttaacgttgaaggcattctttgtctgccgattcaggacagcgacaaaagccatatctggctactggtcaatgacgaccagcgtctggagcagatgataagccaaatcgataagctggaagatgtcgtgaaagtgcagcgtaatcagtccgatccgacgatgtttaacaagatcgcggtgttttttcagtaa

SEQ ID NO: 10 - protein, 96 aa, Escherichia coli
MQNTTHDNVILELTVRNHPGVMTHVCGLFARRAFNVEGILCLPIQDSDKSHIWLLVNDDQRLEQMISQIDKLEDVVKVQRNQSDPTMFNKIAVFFQ

SEQ ID NO: 11 - DNA, 291 bp, synthetic construct
  misc_feature 1..291: note = ilvN* gene
  source 1..291: mol_type = other DNA
atgcaaaacacaactcatgacaacgtaattctggagctcaccgttcgcaaccatccggatgacttcacccacgtttgtggcctttttgcccgccgcgcttttaacgttgaaggcattctttgtctgccgattcaggacagcgacaaaagccatatctggctactggtcaatgacgaccagcgtctggagcagatgataagccaaatcgataagctggaagatgtcgtgaaagtgcagcgtaatcagtccgatccgacgatgtttaacaagatcgcggtgttttttcagtaa

SEQ ID NO: 12 - protein, 96 aa, synthetic construct
  REGION 1..96: note = IlvN* protein
MQNTTHDNVILELTVRNHPDDFTHVCGLFARRAFNVEGILCLPIQDSDKSHIWLLVNDDQRLEQMISQIDKLEDVVKVQRNQSDPTMFNKIAVFFQ

SEQ ID NO: 13 - DNA, 714 bp, Phage lambda
  source 1..714: mol_type = genomic DNA
atgagcacaaaaaagaaaccattaacacaagagcagcttgaggacgcacgtcgccttaaagcaatttatgaaaaaaagaaaaatgaacttggcttatcccaggaatctgtcgcagacaagatggggatggggcagtcaggcgttggtgctttatttaatggcatcaatgcattaaatgcttataacgccgcattgcttacaaaaattctcaaagttagcgttgaagaatttagcccttcaatcgccagagaaatctacgagatgtatgaagcggttagtatgcagccgtcacttagaagtgagtatgagtaccctgttttttctcatgttcaggcagggatgttctcacctaagcttagaacctttaccaaaggtgatgcggagagatgggtaagcacaaccaaaaaagccagtgattctgcattctggcttgaggttgaaggtaattccatgaccgcaccaacaggctccaagccaagctttcctgacggaatgttaattctcgttgaccctgagcaggctgttgagccaggtgatttctgcatagccagacttgggggtgatgagtttaccttcaagaaactgatcagggatagcggtcaggtgtttttacaaccactaaacccacagtacccaatgatcccatgcaatgagagttgttccgttgtggggaaagttatcgctagtcagtggcctgaagagacgtttggctga

SEQ ID NO: 14 - protein, 237 aa, Phage lambda
MSTKKKPLTQEQLEDARRLKAIYEKKKNELGLSQESVADKMGMGQSGVGALFNGINALNAYNAALLTKILKVSVEEFSPSIAREIYEMYEAVSMQPSLRSEYEYPVFSHVQAGMFSPKLRTFTKGDAERWVSTTKKASDSAFWLEVEGNSMTAPTGSKPSFPDGMLILVDPEQAVEPGDFCIARLGGDEFTFKKLIRDSGQVFLQPLNPQYPMIPCNESCSVVGKVIASQWPEETFG

SEQ ID NO: 15 - DNA, 930 bp, Escherichia coli
  source 1..930: mol_type = genomic DNA
atgaccacgaagaaagctgattacatttggttcaatggggagatggttcgctgggaagacgcgaaggtgcatgtgatgtcgcacgcgctgcactatggcacttcggtttttgaaggcatccgttgctacgactcgcacaaaggaccggttgtattccgccatcgtgagcatatgcagcgtctgcatgactccgccaaaatctatcgcttcccggtttcgcagagcattgatgagctgatggaagcttgtcgtgacgtgatccgcaaaaacaatctcaccagcgcctatatccgtccgctgatcttcgtcggtgatgttggcatgggagtaaacccgccagcgggatactcaaccgacgtgattatcgctgctttcccgtggggagcgtatctgggcgcagaagcgctggagcaggggatcgatgcgatggtttcctcctggaaccgcgcagcaccaaacaccatcccgacggcggcaaaagccggtggtaactacctctcttccctgctggtgggtagcgaagcgcgccgccacggttatcaggaaggtatcgcgctggatgtgaacggttatatctctgaaggcgcaggcgaaaacctgtttgaagtgaaagatggtgtgctgttcaccccaccgttcacctcctccgcgctgccgggtattacccgtgatgccatcatcaaactggcgaaagagctgggaattgaagtacgtgagcaggtgctgtcgcgcgaatccctgtacctggcggatgaagtgtttatgtccggtacggcggcagaaatcacgccagtgcgcagcgtagacggtattcaggttggcgaaggccgttgtggcccggttaccaaacgcattcagcaagccttcttcggcctcttcactggcgaaaccgaagataaatggggctggttagatcaagttaatcaataa

SEQ ID NO: 16 - protein, 309 aa, Escherichia coli
MTTKKADYIWFNGEMVRWEDAKVHVMSHALHYGTSVFEGIRCYDSHKGPVVFRHREHMQRLHDSAKIYRFPVSQSIDELMEACRDVIRKNNLTSAYIRPLIFVGDVGMGVNPPAGYSTDVIIAAFPWGAYLGAEALEQGIDAMVSSWNRAAPNTIPTAAKAGGNYLSSLLVGSEARRHGYQEGIALDVNGYISEGAGENLFEVKDGVLFTPPFTSSALPGITRDAIIKLAKELGIEVREQVLSRESLYLADEVFMSGTAAEITPVRSVDGIQVGEGRCGPVTKRIQQAFFGLFTGETEDKWGWLDQVNQ

SEQ ID NO: 17 - DNA, 1851 bp, Escherichia coli
  source 1..1851: mol_type = genomic DNA
atgcctaagtaccgttccgccaccaccactcatggtcgtaatatggcgggtgctcgtgcgctgtggcgcgccaccggaatgaccgacgccgatttcggtaagccgattatcgcggttgtgaactcgttcacccaatttgtaccgggtcacgtccatctgcgcgatctcggtaaactggtcgccgaacaaattgaagcggctggcggcgttgccaaagagttcaacaccattgcggtggatgatgggattgccatgggccacggggggatgctttattcactgccatctcgcgaactgatcgctgattccgttgagtatatggtcaacgcccactgcgccgacgccatggtctgcatctctaactgcgacaaaatcaccccggggatgctgatggcttccctgcgcctgaatattccggtgatctttgtttccggcggcccgatggaggccgggaaaaccaaactttccgatcagatcatcaagctcgatctggttgatgcgatgatccagggcgcagacccgaaagtatctgactcccagagcgatcaggttgaacgttccgcgtgtccgacctgcggttcctgctccgggatgtttaccgctaactcaatgaactgcctgaccgaagcgctgggcctgtcgcagccgggcaacggctcgctgctggcaacccacgccgaccgtaagcagctgttccttaatgctggtaaacgcattgttgaattgaccaaacgttattacgagcaaaacgacgaaagtgcactgccgcgtaatatcgccagtaaggcggcgtttgaaaacgccatgacgctggatatcgcgatgggtggatcgactaacaccgtacttcacctgctggcggcggcgcaggaagcggaaatcgacttcaccatgagtgatatcgataagctttcccgcaaggttccacagctgtgtaaagttgcgccgagcacccagaaataccatatggaagatgttcaccgtgctggtggtgttatcggtattctcggcgaactggatcgcgcggggttactgaaccgtgatgtgaaaaacgtacttggcctgacgttgccgcaaacgctggaacaatacgacgttatgctgacccaggatgacgcggtaaaaaatatgttccgcgcaggtcctgcaggcattcgtaccacacaggcattctcgcaagattgccgttgggatacgctggacgacgatcgcgccaatggctgtatccgctcgctggaacacgcctacagcaaagacggcggcctggcggtgctctacggtaactttgcggaaaacggctgcatcgtgaaaacggcaggcgtcgatgacagcatcctcaaattcaccggcccggcgaaagtgtacgaaagccaggacgatgcggtagaagcgattctcggcggtaaagttgtcgccggagatgtggtagtaattcgctatgaaggcccgaaaggcggtccggggatgcaggaaatgctctacccaaccagcttcctgaaatcaatgggtctcggcaaagcctgtgcgctgatcaccgacggtcgtttctctggtggcacctctggtctttccatcggccacgtctcaccggaagcggcaagcggcggcagcattggcctgattgaagatggtgacctgatcgctatcgacatcccgaaccgtggcattcagttacaggtaagcgatgccgaactggcggcgcgtcgtgaagcgcaggacgctcgaggtgacaaagcctggacgccgaaaaatcgtgaacgtcaggtctcctttgccctgcgtgcttatgccagcctggcaaccagcgccgacaaaggcgcggtgcgcgataaatcgaaactggggggttaa

SEQ ID NO: 18 - protein, 616 aa, Escherichia coli
MPKYRSATTTHGRNMAGARALWRATGMTDADFGKPIIAVVNSFTQFVPGHVHLRDLGKLVAEQIEAAGGVAKEFNTIAVDDGIAMGHGGMLYSLPSRELIADSVEYMVNAHCADAMVCISNCDKITPGMLMASLRLNIPVIFVSGGPMEAGKTKLSDQIIKLDLVDAMIQGADPKVSDSQSDQVERSACPTCGSCSGMFTANSMNCLTEALGLSQPGNGSLLATHADRKQLFLNAGKRIVELTKRYYEQNDESALPRNIASKAAFENAMTLDIAMGGSTNTVLHLLAAAQEAEIDFTMSDIDKLSRKVPQLCKVAPSTQKYHMEDVHRAGGVIGILGELDRAGLLNRDVKNVLGLTLPQTLEQYDVMLTQDDAVKNMFRAGPAGIRTTQAFSQDCRWDTLDDDRANGCIRSLEHAYSKDGGLAVLYGNFAENGCIVKTAGVDDSILKFTGPAKVYESQDDAVEAILGGKVVAGDVVVIRYEGPKGGPGMQEMLYPTSFLKSMGLGKACALITDGRFSGGTSGLSIGHVSPEAASGGSIGLIEDGDLIAIDIPNRGIQLQVSDAELAARREAQDARGDKAWTPKNRERQVSFALRAYASLATSADKGAVRDKSKLGG

SEQ ID NO: 19 - DNA, 1476 bp, Escherichia coli
  source 1..1476: mol_type = genomic DNA
atggctaactacttcaatacactgaatctgcgccagcagctggcacagctgggcaaatgtcgctttatgggccgcgatgaattcgccgatggcgcgagctaccttcagggtaaaaaagtagtcatcgtcggctgtggcgcacagggtctgaaccagggcctgaacatgcgtgattctggtctcgatatctcctacgctctgcgtaaagaagcgattgccgagaagcgcgcgtcctggcgtaaagcgaccgaaaatggttttaaagtgggtacttacgaagaactgatcccacaggcggatctggtgattaacctgacgccggacaagcagcactctgatgtagtgcgcaccgtacagccactgatgaaagacggcgcggcgctgggctactcgcacggtttcaacatcgtcgaagtgggcgagcagatccgtaaagatatcaccgtagtgatggttgcgccgaaatgcccaggcaccgaagtgcgtgaagagtacaaacgtgggttcggcgtaccgacgctgattgccgttcacccggaaaacgatccgaaaggcgaaggcatggcgattgccaaagcctgggcggctgcaaccggtggtcaccgtgcgggtgtgctggaatcgtccttcgttgcggaagtgaaatctgacctgatgggcgagcaaaccatcctgtgcggtatgttgcaggctggctctctgctgtgcttcgacaagctggtggaagaaggtaccgatccagcatacgcagaaaaactgattcagttcggttgggaaaccatcaccgaagcactgaaacagggcggcatcaccctgatgatggaccgtctctctaacccggcgaaactgcgtgcttatgcgctttctgaacagctgaaagagatcatggcacccctgttccagaaacatatggacgacatcatctccggcgaattctcttccggtatgatggcggactgggccaacgatgataagaaactgctgacctggcgtgaagagaccggcaaaaccgcgtttgaaaccgcgccgcagtatgaaggcaaaatcggcgagcaggagtacttcgataaaggcgtactgatgattgcgatggtgaaagcgggcgttgaactggcgttcgaaaccatggtcgattccggcatcattgaagagtctgcatattatgaatcactgcacgagctgccgctgattgccaacaccatcgcccgtaagcgtctgtacgaaatgaacgtggttatctctgataccgctgagtacggtaactatctgttctcttacgcttgtgtgccgttgctgaaaccgtttatggcagagctgcaaccgggcgacctgggtaaagctattccggaaggcgcggtagataacgggcaactgcgtgatgtgaacgaagcgattcgcagccatgcgattgagcaggtaggtaagaaactgcgcggctatatgacagatatgaaacgtattgctgttgcgggttaa

SEQ ID NO: 20 - protein, 491 aa, Escherichia coli
MANYFNTLNLRQQLAQLGKCRFMGRDEFADGASYLQGKKVVIVGCGAQGLNQGLNMRDSGLDISYALRKEAIAEKRASWRKATENGFKVGTYEELIPQADLVINLTPDKQHSDVVRTVQPLMKDGAALGYSHGFNIVEVGEQIRKDITVVMVAPKCPGTEVREEYKRGFGVPTLIAVHPENDPKGEGMAIAKAWAAATGGHRAGVLESSFVAEVKSDLMGEQTILCGMLQAGSLLCFDKLVEEGTDPAYAEKLIQFGWETITEALKQGGITLMMDRLSNPAKLRAYALSEQLKEIMAPLFQKHMDDIISGEFSSGMMADWANDDKKLLTWREETGKTAFETAPQYEGKIGEQEYFDKGVLMIAMVKAGVELAFETMVDSGIIEESAYYESLHELPLIANTIARKRLYEMNVVISDTAEYGNYLFSYACVPLLKPFMAELQPGDLGKAIPEGAVDNGQLRDVNEAIRSHAIEQVGKKLRGYMTDMKRIAVAG

SEQ ID NO: 21 - DNA, 1572 bp, Escherichia coli
  source 1..1572: mol_type = genomic DNA
atgagccagcaagtcattattttcgataccacattgcgcgacggtgaacaggcgttacaggcaagcttgagtgtgaaagaaaaactgcaaattgcgctggcccttgagcgtatgggtgttgacgtgatggaagtcggtttccccgtctcttcgccgggcgattttgaatcggtgcaaaccatcgcccgccaggttaaaaacagccgcgtatgtgcgttagctcgctgcgtggaaaaagatatcgacgtggcggccgaatccctgaaagtcgccgaagccttccgtattcatacctttattgccacttcgccaatgcacatcgccaccaagctgcgcagcacgctggacgaggtgatcgaacgcgctatctatatggtgaaacgcgcccgtaattacaccgatgatgttgaattttcttgcgaagatgccgggcgtacacccattgccgatctggcgcgagtggtcgaagcggcgattaatgccggtgccaccaccatcaacattccggacaccgtgggctacaccatgccgtttgagttcgccggaatcatcagcggcctgtatgaacgcgtgcctaacatcgacaaagccattatctccgtacatacccacgacgatttgggcctggcggtcggaaactcactggcggcggtacatgccggtgcacgccaggtggaaggcgcaatgaacgggatcggcgagcgtgccggaaactgttccctggaagaagtcatcatggcgatcaaagttcgtaaggatattctcaacgtccacaccgccattaatcaccaggagatatggcgcaccagccagttagttagccagatttgtaatatgccgatcccggcaaacaaagccattgttggcagcggcgcattcgcacactcctccggtatacaccaggatggcgtgctgaaaaaccgcgaaaactacgaaatcatgacaccagaatctattggtctgaaccaaatccagctgaatctgacctctcgttcggggcgtgcggcggtgaaacatcgcatggatgagatggggtataaagaaagtgaatataatttagacaatttgtacgatgctttcctgaagctggcggacaaaaaaggtcaggtgtttgattacgatctggaggcgctggccttcatcggtaagcagcaagaagagccggagcatttccgtctggattacttcagcgtgcagtctggctctaacgatatcgccaccgccgccgtcaaactggcctgtggcgaagaagtcaaagcagaagccgccaacggtaacggtccggtcgatgccgtctatcaggcaattaaccgcatcactgaatataacgtcgaactggtgaaatacagcctgaccgccaaaggccacggtaaagatgcgctgggtcaggtggatatcgtcgctaactacaacggtcgccgcttccacggcgtcggcctggctaccgatattgtcgagtcatctgccaaagccatggtgcacgttctgaacaatatctggcgtgccgcagaagtcgaaaaagagttgcaacgcaaagctcaacacaacgaaaacaacaaggaaaccgtgtga

SEQ ID NO: 22 - protein, 523 aa, Escherichia coli
MSQQVIIFDTTLRDGEQALQASLSVKEKLQIALALERMGVDVMEVGFPVSSPGDFESVQTIARQVKNSRVCALARCVEKDIDVAAESLKVAEAFRIHTFIATSPMHIATKLRSTLDEVIERAIYMVKRARNYTDDVEFSCEDAGRTPIADLARVVEAAINAGATTINIPDTVGYTMPFEFAGIISGLYERVPNIDKAIISVHTHDDLGLAVGNSLAAVHAGARQVEGAMNGIGERAGNCSLEEVIMAIKVRKDILNVHTAINHQEIWRTSQLVSQICNMPIPANKAIVGSGAFAHSSGIHQDGVLKNRENYEIMTPESIGLNQIQLNLTSRSGRAAVKHRMDEMGYKESEYNLDNLYDAFLKLADKKGQVFDYDLEALAFIGKQQEEPEHFRLDYFSVQSGSNDIATAAVKLACGEEVKAEAANGNGPVDAVYQAINRITEYNVELVKYSLTAKGHGKDALGQVDIVANYNGRRFHGVGLATDIVESSAKAMVHVLNNIWRAAEVEKELQRKAQHNENNKETV

SEQ ID NO: 23 - DNA, 1572 bp, synthetic construct
  misc_feature 1..1572: note = leuA* gene
  source 1..1572: mol_type = other DNA
atgagccagcaagtcattattttcgataccacattgcgcgacggtgaacaggcgttacaggcaagcttgagtgtgaaagaaaaactgcaaattgcgctggcccttgagcgtatgggtgttgacgtgatggaagtcggtttccccgtctcttcgccgggcgattttgaatcggtgcaaaccatcgcccgccaggttaaaaacagccgcgtatgtgcgttagctcgctgcgtggaaaaagatatcgacgtggcggccgaatccctgaaagtcgccgaagccttccgtattcatacctttattgccacttcgccaatgcacatcgccaccaagctgcgcagcacgctggacgaggtgatcgaacgcgctatctatatggtgaaacgcgcccgtaattacaccgatgatgttgaattttcttgcgaagatgccgggcgtacacccattgccgatctggcgcgagtggtcgaagcggcgattaatgccggtgccaccaccatcaacattccggacaccgtgggctacaccatgccgtttgagttcgccggaatcatcagcggcctgtatgaacgcgtgcctaacatcgacaaagccattatctccgtacatacccacgacgatttgggcctggcggtcggaaactcactggcggcggtacatgccggtgcacgccaggtggaaggcgcaatgaacgggatcggcgagcgtgccggaaactgttccctggaagaagtcatcatggcgatcaaagttcgtaaggatattctcaacgtccacaccgccattaatcaccaggagatatggcgcaccagccagttagttagccagatttgtaatatgccgatcccggcaaacaaagccattgttggcagcggcgcattcgcacactcctccggtatacaccaggatggcgtgctgaaaaaccgcgaaaactacgaaatcatgacaccagaatctattggtctgaaccaaatccagctgaatctgacctctcgttcggggcgtgcggcggtgaaacatcgcatggatgagatggggtataaagaaagtgaatataatttagacaatttgtacgatgctttcctgaagctggcggacaaaaaaggtcaggtgtttgattacgatctggaggcgctggccttcatcggtaagcagcaagaagagccggagcatttccgtctggattacttcagcgtgcagtctggctctaacgatatcgccaccgccgccgtcaaactggcctgtggcgaagaagtcaaagcagaagccgccaacggtaacggtccggtcgatgccgtctatcaggcaattaaccgcatcactgaatataacgtcgaactggtgaaatacagcctgaccgccaaaggccacggtaaagatgcgctggaccaggtggatatcgtcgctaactacaacggtcgccgcttccacggcgtcggcctggctaccgatattgtcgagtcatctgccaaagccatggtgcacgttctgaacaatatctggcgtgccgcagaagtcgaaaaagagttgcaacgcaaagctcaacacaacgaaaacaacaaggaaaccgtgtga

SEQ ID NO: 24 - protein, 523 aa, synthetic construct
  REGION 1..523: note = LeuA* protein
MSQQVIIFDTTLRDGEQALQASLSVKEKLQIALALERMGVDVMEVGFPVSSPGDFESVQTIARQVKNSRVCALARCVEKDIDVAAESLKVAEAFRIHTFIATSPMHIATKLRSTLDEVIERAIYMVKRARNYTDDVEFSCEDAGRTPIADLARVVEAAINAGATTINIPDTVGYTMPFEFAGIISGLYERVPNIDKAIISVHTHDDLGLAVGNSLAAVHAGARQVEGAMNGIGERAGNCSLEEVIMAIKVRKDILNVHTAINHQEIWRTSQLVSQICNMPIPANKAIVGSGAFAHSSGIHQDGVLKNRENYEIMTPESIGLNQIQLNLTSRSGRAAVKHRMDEMGYKESEYNLDNLYDAFLKLADKKGQVFDYDLEALAFIGKQQEEPEHFRLDYFSVQSGSNDIATAAVKLACGEEVKAEAANGNGPVDAVYQAINRITEYNVELVKYSLTAKGHGKDALDQVDIVANYNGRRFHGVGLATDIVESSAKAMVHVLNNIWRAAEVEKELQRKAQHNENNKETV

SEQ ID NO: 25 - DNA, 1092 bp, Escherichia coli
  source 1..1092: mol_type = genomic DNA
atgtcgaagaattaccatattgccgtattgccgggggacggtattggtccggaagtgatgacccaggcgctgaaagtgctggatgccgtgcgcaaccgctttgcgatgcgcatcaccaccagccattacgatgtaggcggcgcagccattgataaccacgggcaaccactgccgcctgcgacggttgaaggttgtgagcaagccgatgccgtgctgtttggctcggtaggcggcccgaagtgggaacatttaccaccagaccagcaaccagaacgcggcgcgctgctgcctctgcgtaagcacttcaaattattcagcaacctgcgcccggcaaaactgtatcaggggctggaagcattctgtccgctgcgtgcagacattgccgcaaacggcttcgacatcctgtgtgtgcgcgaactgaccggcggcatctatttcggtcagccaaaaggccgcgaaggtagcggacaatatgaaaaagcctttgataccgaggtgtatcaccgttttgagatcgaacgtatcgcccgcatcgcgtttgaatctgctcgcaagcgtcgccacaaagtgacgtcgatcgataaagccaacgtgctgcaatcctctattttatggcgggagatcgttaacgagatcgccacggaatacccggatgtcgaactggcgcatatgtacatcgacaacgccaccatgcagctgattaaagatccatcacagtttgacgttctgctgtgctccaacctgtttggcgacattctgtctgacgagtgcgcaatgatcactggctcgatggggatgttgccttccgccagcctgaacgagcaaggttttggactgtatgaaccggcgggcggctcggcaccagatatcgcaggcaaaaacatcgccaacccgattgcacaaatcctttcgctggcactgctgctgcgttacagcctggatgccgatgatgcggcttgcgccattgaacgcgccattaaccgcgcattagaagaaggcattcgcaccggggatttagcccgtggcgctgccgccgttagtaccgatgaaatgggcgatatcattgcccgctatgtagcagaaggggtgtaa

SEQ ID NO: 26 - protein, 363 aa, Escherichia coli
MSKNYHIAVLPGDGIGPEVMTQALKVLDAVRNRFAMRITTSHYDVGGAAIDNHGQPLPPATVEGCEQADAVLFGSVGGPKWEHLPPDQQPERGALLPLRKHFKLFSNLRPAKLYQGLEAFCPLRADIAANGFDILCVRELTGGIYFGQPKGREGSGQYEKAFDTEVYHRFEIERIARIAFESARKRRHKVTSIDKANVLQSSILWREIVNEIATEYPDVELAHMYIDNATMQLIKDPSQFDVLLCSNLFGDILSDECAMITGSMGMLPSASLNEQGFGLYEPAGGSAPDIAGKNIANPIAQILSLALLLRYSLDADDAACAIERAINRALEEGIRTGDLARGAAAVSTDEMGDIIARYVAEGV

SEQ ID NO: 27 - DNA, 1401 bp, Escherichia coli
  source 1..1401: mol_type = genomic DNA
atggctaagacgttatacgaaaaattgttcgacgctcacgttgtgtacgaagccgaaaacgaaaccccactgttatatatcgaccgccacctggtgcatgaagtgacctcaccgcaggcgttcgatggtctgcgcgcccacggtcgcccggtacgtcagccgggcaaaaccttcgctaccatggatcacaacgtctctacccagaccaaagacattaatgcctgcggtgaaatggcgcgtatccagatgcaggaactgatcaaaaactgcaaagaatttggcgtcgaactgtatgacctgaatcacccgtatcaggggatcgtccacgtaatggggccggaacagggcgtcaccttgccggggatgaccattgtctgcggcgactcgcataccgccacccacggcgcgtttggcgcactggcctttggtatcggcacttccgaagttgaacacgtactggcaacgcaaaccctgaaacagggccgcgcaaaaaccatgaaaattgaagtccagggcaaagccgcgccgggcattaccgcaaaagatatcgtgctggcaattatcggtaaaaccggtagcgcaggcggcaccgggcatgtggtggagttttgcggcgaagcaatccgtgatttaagcatggaaggtcgtatgaccctgtgcaatatggcaatcgaaatgggcgcaaaagccggtctggttgcaccggacgaaaccacctttaactatgtcaaaggccgtctgcatgcgccgaaaggcaaagatttcgacgacgccgttgcctactggaaaaccctgcaaaccgacgaaggcgcaactttcgataccgttgtcactctgcaagcagaagaaatttcaccgcaggtcacctggggcaccaatcccggccaggtgatttccgtgaacgacaatattcccgatccggcttcgtttgccgatccggttgaacgcgcgtcggcagaaaaagcgctggcctatatggggctgaaaccgggtattccgctgaccgaagtggctatcgacaaagtgtttatcggttcctgtaccaactcgcgcattgaagatttacgcgcggcagcggagatcgccaaagggcgaaaagtcgcgccaggcgtgcaggcactggtggttcccggctctggcccggtaaaagcccaggcggaagcggaaggtctggataaaatctttattgaagccggttttgaatggcgcttgcctggctgctcaatgtgtctggcgatgaacaacgaccgtctgaatccgggcgaacgttgtgcctccaccagcaaccgtaactttgaaggccgccaggggcgcggcgggcgcacgcatctggtcagcccggcaatggctgccgctgctgctgtgaccggacatttcgccgacattcgcaacattaaataa

SEQ ID NO: 28 - protein, 466 aa, Escherichia coli
MAKTLYEKLFDAHVVYEAENETPLLYIDRHLVHEVTSPQAFDGLRAHGRPVRQPGKTFATMDHNVSTQTKDINACGEMARIQMQELIKNCKEFGVELYDLNHPYQGIVHVMGPEQGVTLPGMTIVCGDSHTATHGAFGALAFGIGTSEVEHVLATQTLKQGRAKTMKIEVQGKAAPGITAKDIVLAIIGKTGSAGGTGHVVEFCGEAIRDLSMEGRMTLCNMAIEMGAKAGLVAPDETTFNYVKGRLHAPKGKDFDDAVAYWKTLQTDEGATFDTVVTLQAEEISPQVTWGTNPGQVISVNDNIPDPASFADPVERASAEKALAYMGLKPGIPLTEVAIDKVFIGSCTNSRIEDLRAAAEIAKGRKVAPGVQALVVPGSGPVKAQAEAEGLDKIFIEAGFEWRLPGCSMCLAMNNDRLNPGERCASTSNRNFEGRQGRGGRTHLVSPAMAAAAAVTGHFADIRNIK

SEQ ID NO: 29 - DNA, 606 bp, Escherichia coli
  source 1..606: mol_type = genomic DNA
atggcagagaaatttatcaaacacacaggcctggtggttccgctggatgccgccaatgtcgataccgatgcaatcatcccgaaacagtttttgcagaaagtgacccgtacgggttttggcgcgcatctgtttaacgactggcgttttctggatgaaaaaggccaacagccaaacccggacttcgtgctgaacttcccgcagtatcagggcgcttccattttgctggcacgagaaaacttcggctgtggctcttcgcgtgagcacgcgccctgggcattgaccgactacggttttaaagtggtgattgcgccgagttttgctgacatcttctacggcaatagctttaacaaccagctgctgccggtgaaattaagcgatgcagaagtggacgaactgtttgcgctggtgaaagctaatccggggatccatttcgacgtggatctggaagcgcaagaggtgaaagcgggagagaaaacctatcgctttaccatcgatgccttccgccgccactgcatgatgaacggtctggacagtattgggcttaccttgcagcacgacgacgccattgccgcttatgaagcaaaacaacctgcgtttatgaattaa

SEQ ID NO: 30 - protein, 201 aa, Escherichia coli
MAEKFIKHTGLVVPLDAANVDTDAIIPKQFLQKVTRTGFGAHLFNDWRFLDEKGQQPNPDFVLNFPQYQGASILLARENFGCGSSREHAPWALTDYGFKVVIAPSFADIFYGNSFNNQLLPVKLSDAEVDELFALVKANPGIHFDVDLEAQEVKAGEKTYRFTIDAFRRHCMMNGLDSIGLTLQHDDAIAAYEAKQPAFMN

SEQ ID NO: 31 - DNA, 1284 bp, Escherichia coli
  source 1..1284: mol_type = genomic DNA
atggctgatacaaaagcaaaactcaccctcaacggggatacagctgttgaactggatgtgctgaaaggcacgctgggtcaagatgttattgatatccgtactctcggttcaaaaggtgtgttcacctttgacccaggcttcacttcaaccgcatcctgcgaatctaaaattacttttattgatggtgatgaaggtattttgctgcaccgcggtttcccgatcgatcagctggcgaccgattctaactacctggaagtttgttacatcctgctgaatggtgaaaaaccgactcaggaacagtatgacgaatttaaaactacggtgacccgtcataccatgatccacgagcagattacccgtctgttccatgctttccgtcgcgactcgcatccaatggcagtcatgtgtggtattaccggcgcgctggcggcgttctatcacgactcgctggatgttaacaatcctcgtcaccgtgaaattgccgcgttccgcctgctgtcgaaaatgccgaccatggccgcgatgtgttacaagtattccattggtcagccatttgtttacccgcgcaacgatctctcctacgccggtaacttcctgaatatgatgttctccacgccgtgcgaaccgtatgaagttaatccgattctggaacgtgctatggaccgtattctgatcctgcacgctgaccatgaacagaacgcctctacctccaccgtgcgtaccgctggctcttcgggtgcgaacccgtttgcctgtatcgcagcaggtattgcttcactgtggggacctgcgcacggcggtgctaacgaagcggcgctgaaaatgctggaagaaatcagctccgttaaacacattccggaatttgttcgtcgtgcgaaagacaaaaatgattctttccgcctgatgggcttcggtcaccgcgtgtacaaaaattacgacccgcgcgccaccgtaatgcgtgaaacctgccatgaagtgctgaaagagctgggcacgaaggatgacctgctggaagtggctatggagctggaaaacatcgcgctgaacgacccgtactttatcgagaagaaactgtacccgaacgtcgatttctactctggtatcatcctgaaagcgatgggtattccgtcttccatgttcaccgtcattttcgcaatggcacgtaccgttggctggatcgcccactggagcgaaatgcacagtgacggtatgaagattgcccgtccgcgtcagctgtatacaggatatgaaaaacgcgactttaaaagcgatatcaagcgttaa

SEQ ID NO: 32 - protein, 427 aa, Escherichia coli
MADTKAKLTLNGDTAVELDVLKGTLGQDVIDIRTLGSKGVFTFDPGFTSTASCESKITFIDGDEGILLHRGFPIDQLATDSNYLEVCYILLNGEKPTQEQYDEFKTTVTRHTMIHEQITRLFHAFRRDSHPMAVMCGITGALAAFYHDSLDVNNPRHREIAAFRLLSKMPTMAAMCYKYSIGQPFVYPRNDLSYAGNFLNMMFSTPCEPYEVNPILERAMDRILILHADHEQNASTSTVRTAGSSGANPFACIAAGIASLWGPAHGGANEAALKMLEEISSVKHIPEFVRRAKDKNDSFRLMGFGHRVYKNYDPRATVMRETCHEVLKELGTKDDLLEVAMELENIALNDPYFIEKKLYPNVDFYSGIILKAMGIPSSMFTVIFAMARTVGWIAHWSEMHSDGMKIARPRQLYTGYEKRDFKSDIKR

SEQ ID NO: 33 - DNA, 996 bp, Escherichia coli
  source 1..996: mol_type = genomic DNA
atgactatcaaagtaggtatcaacggttttggccgtatcggtcgcattgttttccgtgctgctcagaaacgttctgacatcgagatcgttgcaatcaacgacctgttagacgctgattacatggcatacatgctgaaatatgactccactcacggccgtttcgacggtaccgttgaagtgaaagacggtcatctgatcgttaacggtaaaaaaatccgtgttaccgctgaacgtgatccggctaacctgaaatgggacgaagttggtgttgacgttgtcgctgaagcaactggtctgttcctgactgacgaaactgctcgtaaacacatcaccgctggtgcgaagaaagtggttatgactggtccgtctaaagacaacactccgatgttcgttaaaggcgctaacttcgacaaatatgctggccaggacatcgtttccaacgcttcctgcaccaccaactgcctggctccgctggctaaagttatcaacgataacttcggcatcatcgaaggtctgatgaccaccgttcacgctactaccgctactcagaaaaccgttgatggcccgtctcacaaagactggcgcggcggccgcggcgcttcccagaacatcatcccgtcctctaccggtgctgctaaagctgtaggtaaagtactgccagaactgaatggcaaactgactggtatggcgttccgcgttccgaccccgaacgtatctgtagttgacctgaccgttcgtctggaaaaagctgcaacttacgagcagatcaaagctgccgttaaagctgctgctgaaggcgaaatgaaaggcgttctgggctacaccgaagatgacgtagtatctaccgatttcaacggcgaagtttgcacttccgtgttcgatgctaaagctggtatcgctctgaacgacaacttcgtgaaactggtatcctggtacgacaacgaaaccggttactccaacaaagttctggacctgatcgctcacatctccaaataa

SEQ ID NO: 34 - protein, 331 aa, Escherichia coli
MTIKVGINGFGRIGRIVFRAAQKRSDIEIVAINDLLDADYMAYMLKYDSTHGRFDGTVEVKDGHLIVNGKKIRVTAERDPANLKWDEVGVDVVAEATGLFLTDETARKHITAGAKKVVMTGPSKDNTPMFVKGANFDKYAGQDIVSNASCTTNCLAPLAKVINDNFGIIEGLMTTVHATTATQKTVDGPSHKDWRGGRGASQNIIPSSTGAAKAVGKVLPELNGKLTGMAFRVPTPNVSVVDLTVRLEKAATYEQIKAAVKAAAEGEMKGVLGYTEDDVVSTDFNGEVCTSVFDAKAGIALNDNFVKLVSWYDNETGYSNKVLDLIAHISK

SEQ ID NO: 35 - DNA, 1434 bp, Streptococcus mutans
  source 1..1434: mol_type = genomic DNA
atggcaatgacaaaacaatataaaaattatgtcaatggcgagtggaagctttcagaaaatgaaattaaaatctacgaaccagccagtggagctgaattgggttcagttccagcaatgagtactgaagaagtagattatgtttatgcttcagccaagaaagctcaaccagcttggcgagcactttcatacatagaacgtgctgcctaccttcataaggtagcagatattttgatgcgtgataaagaaaaaataggtgctattctttccaaagaggttgctaaaggttataaatcagcagtcagcgaagttgttcgtactgcagaaatcattaattatgcagctgaagaaggtcttcgtatggaaggtgaagtccttgaaggcggcagttttgaagcagccagcaagaaaaaaattgccgttgttcgtcgtgaaccagtaggtcttgtattagctatttcaccatttaactaccctgttaacttggcaggttcgaaaattgcaccggctcttattgcgggaaatgttattgcttttaaaccaccgacgcaaggatcaatctcagggctcttacttgctgaagcatttgctgaagctggacttcctgcaggtgtctttaataccattacaggtcgtggttctgaaattggagactatattgtagaacatcaagccgttaactttatcaatttcactggttcaacaggaattggcgaacgtattggcaaaatggctggtatgcgtccgattatgcttgaactcggtggaaaagattcagccatcgttcttgaagatgcggaccttgaattgactgctaaaaatattattgcaggtgcttttggttattcaggtcaacgctgtacagcagttaaacgtgttcttgtgatggaaagtgttgctgatgaactggtcgaaaaaatccgtgaaaaagttcttgcattaacaattggtaatccagaagacgatgcagatattacaccgttgattgatacaaaatcagctgattatgtagaaggtcttattaatgatgccaatgataaaggagccactgcccttactgaaatcaaacgtgaaggtaatcttatctgtccaatcctctttgataaggtaacgacagatatgcgtcttgcttgggaagaaccatttggtcctgttcttccgatcattcgtgtgacatctgtagaagaagccattgaaatttctaacaaatcggaatatggacttcaggcttctatctttacaaatgatttcccacgcgcttttggtattgctgagcagcttgaagttggtacagttcatatcaataataagacacagcgcggcacggacaacttcccattcttaggggctaaaaaatcaggtgcaggtattcaaggggtaaaatattctattgaagctatgacaactgttaaatccgtcgtatttgatatcaaataa

SEQ ID NO: 36 - protein, 477 aa, Streptococcus mutans
MAMTKQYKNYVNGEWKLSENEIKIYEPASGAELGSVPAMSTEEVDYVYASAKKAQPAWRALSYIERAAYLHKVADILMRDKEKIGAILSKEVAKGYKSAVSEVVRTAEIINYAAEEGLRMEGEVLEGGSFEAASKKKIAVVRREPVGLVLAISPFNYPVNLAGSKIAPALIAGNVIAFKPPTQGSISGLLLAEAFAEAGLPAGVFNTITGRGSEIGDYIVEHQAVNFINFTGSTGIGERIGKMAGMRPIMLELGGKDSAIVLEDADLELTAKNIIAGAFGYSGQRCTAVKRVLVMESVADELVEKIREKVLALTIGNPEDDADITPLIDTKSADYVEGLINDANDKGATALTEIKREGNLICPILFDKVTTDMRLAWEEPFGPVLPIIRVTSVEEAIEISNKSEYGLQASIFTNDFPRAFGIAEQLEVGTVHINNKTQRGTDNFPFLGAKKSGAGIQGVKYSIEAMTTVKSVVFDIK

SEQ ID NO: 37 - DNA, 1428 bp, Streptococcus pyogenes
  source 1..1428: mol_type = genomic DNA
ttggcaaaacaatataaaaatttagtgaacggtgaatggaaactatcagaaaacgagattaccatttacgcaccagcaacaggtgaagagttaggatcagttccagcgatgacgcaggcagaggtagatgctgtttacgcttcagctaaaaaggctctatcagattggcgcgctttgtcttatgtggaacgtgcagcttaccttcataaagcggctgatattttagtacgtgatgctgaaaagatcggcgcgattctttcaaaagaagtagccaaaggtcacaaggcagctgtcagtgaagttattcgtaccgctgaaatcattaattatgcagcagaagaagggcttcgtatggaaggtgaagttcttgaaggtggtagcttcgaagctgcaagtaagaagaagattgctattgttcgtcgtgaaccagttggtttagttcttgccatctcaccttttaattatcccgttaacttggcaggttctaaaattgctccagctcttattgcaggaaatgttgttgctcttaaaccaccaacacaaggctctatttctggtttgttactagcagaagcttttgcagaagctggtattccagcaggtgtctttaataccattacagggcgaggttctgttatcggtgattatatcgttgagcacgaagcggttagctttatcaactttacaggttctactccaattggggaaggaatcggtaaattagcgggtatgcgaccaattatgcttgagcttggcggtaaggattctgctatcgttttggaagatgcagatcttgctttagcagcgaaaaatattgtagccggtgcttttggttactcaggccaacgttgtacagcggttaaacgtgttcttgtgatggacaaggtggcggatcaattggcggctgagattaaaacacttgttgaaaaactaagtgtcggaatgcctgaagacgatgctgatattacaccattaattgatacatcagctgctgattttgttgaagggttgattaaagatgcaactgataagggagctactgctttgacagcctttaatcgtgaaggcaatcttatttcacccgttctctttgatcatgtgacaactgacatgcgtttggcatgggaagagccgttcggcccagtattaccaattattcgtgtaaccactgtagaagaagccatcaagatttctaatgagtctgaatatggtttgcaagcttctatttttacaactaatttcccaaaagcttttggcattgctgagcaattagaagttggaactgttcaccttaacaataaaacacaacgtggaacagataatttcccattcttaggcgctaaaaaatcaggtgcaggggtacaaggagttaaatattctatcgaagctatgacaactgttaaatctgttgtatttgatatccagtaa

SEQ ID NO: 38 - protein, 475 aa, Streptococcus pyogenes
MAKQYKNLVNGEWKLSENEITIYAPATGEELGSVPAMTQAEVDAVYASAKKALSDWRALSYVERAAYLHKAADILVRDAEKIGAILSKEVAKGHKAAVSEVIRTAEIINYAAEEGLRMEGEVLEGGSFEAASKKKIAIVRREPVGLVLAISPFNYPVNLAGSKIAPALIAGNVVALKPPTQGSISGLLLAEAFAEAGIPAGVFNTITGRGSVIGDYIVEHEAVSFINFTGSTPIGEGIGKLAGMRPIMLELGGKDSAIVLEDADLALAAKNIVAGAFGYSGQRCTAVKRVLVMDKVADQLAAEIKTLVEKLSVGMPEDDADITPLIDTSAADFVEGLIKDATDKGATALTAFNREGNLISPVLFDHVTTDMRLAWEEPFGPVLPIIRVTTVEEAIKISNESEYGLQASIFTTNFPKAFGIAEQLEVGTVHLNNKTQRGTDNFPFLGAKKSGAGVQGVKYSIEAMTTVKSVVFDIQ

SEQ ID NO: 39 - DNA, 1449 bp, Clostridium acetobutylicum
  source 1..1449: mol_type = genomic DNA
atgtttgaaaatatatcatcaaatggagtttataaaaatctatttgatggaaaatgggttgaaagtaagacaaataaaaccatagaaacgcattctccttatgatggaagtttaattggaaaagttcaggccttatcaaaagaggaagttgatgagatttttaaaagttcaagaacagctcagaaaaaatggggtgaaactccaataaatgagcgtgctagaatcatgcgtaaagcagctgatatactagatgataacgcagaatatatagcaaaaattctttcaaatgagatagcaaaagatttaaaatcttctctttcagaagtaaaaagaacagctgattttataagatttacagctaatgaaggtactcatatggaaggagaagctattaactcagataattttcctggttctaaaaaagataaactttctctagttgaaagagttcctttaggaatagttttagctatatctccttttaattatcctgtaaatctttctgggtctaaggttgctccagcacttatagctggaaatagtgttgttttaaaaccttctacaactggtgctataagcgcacttcatcttgcagaaatttttaatgcagctggtcttccagcaggtgttttaaacactgtaacaggaaaagggtctgaaataggcgattatttaattacccatgaagaagtaaactttattaactttacgggaagctctgctgtaggtaagcatatttcaaaaatagctggaatgatacctatggttcttgagcttggtggtaaagatgctgctatagttctcgaagatgccaatcttgaaacaacagctaaaagcatagtatctggagcatatggatactccggccaaaggtgtactgctgtaaaaagagttcttgtaatggataaagtagctgatgaattagttgaacttgttacaaaaaaagttaaagaattaaaggtaggtaatccttttgatgatgttacaataaccccacttatagacaacaaggcagcagattatgttcaaactctcattgacgacgctatcgaaaagggtgcaactcttatcgttggaaataagcgtaaagaaaatttaatgtatcctactttatttgataatgtaactgctgatatgcgtattgcttgggaagaaccatttggaccagttttacctattattcgtgtaaaaagcatggatgaagcaatagaattagcaaatagatctgaatatggtcttcaatctgcagtatttactgaaaatatgcatgatgccttttatattgccaataaattagatgttggaactgttcaagtaaataataagcctgaaagaggcccagatcacttcccattccttggaacaaagtcatcaggtatgggcactcaaggaattcgatacagtatagaggcaatgacaaggcataaatcaatagttttaaacctataa

SEQ ID NO: 40 - protein, 482 aa, Clostridium acetobutylicum
MFENISSNGVYKNLFDGKWVESKTNKTIETHSPYDGSLIGKVQALSKEEVDEIFKSSRTAQKKWGETPINERARIMRKAADILDDNAEYIAKILSNEIAKDLKSSLSEVKRTADFIRFTANEGTHMEGEAINSDNFPGSKKDKLSLVERVPLGIVLAISPFNYPVNLSGSKVAPALIAGNSVVLKPSTTGAISALHLAEIFNAAGLPAGVLNTVTGKGSEIGDYLITHEEVNFINFTGSSAVGKHISKIAGMIPMVLELGGKDAAIVLEDANLETTAKSIVSGAYGYSGQRCTAVKRVLVMDKVADELVELVTKKVKELKVGNPFDDVTITPLIDNKAADYVQTLIDDAIEKGATLIVGNKRKENLMYPTLFDNVTADMRIAWEEPFGPVLPIIRVKSMDEAIELANRSEYGLQSAVFTENMHDAFYIANKLDVGTVQVNNKPERGPDHFPFLGTKSSGMGTQGIRYSIEAMTRHKSIVLNL

SEQ ID NO: 41 - DNA, 1440 bp, Bacillus cereus
  source 1..1440: mol_type = genomic DNA
atgacaactagcaatacgtacaaattttatttaaatggagaatggagagaaagctcttcaggagaaacaatcgaaattccatctccatacttacacgaagtaattggacaagtacaagcaattactcgcggagaagtagatgaagcaattgcatctgcaaaagaagctcaaaaatcatgggcagaagcttctctacaagatcgcgcaaagtatttatataaatgggctgatgagctcgtaaatatgcaggatgaaattgccgatatcattatgaaagaagtcggtaaaggctataaagatgcgaaaaaagaagttgttcgtacagctgacttcattcgttacacgatcgaagaagctttacatatgcacggagaaagcatgatgggcgatagcttcccgggcggtacaaaatctaaacttgcgattatccaacgcgcaccacttggtgttgtattagcaatcgcaccgtttaactacccagtaaacttatccgctgcaaaacttgcaccagcattaattatgggtaacgctgttattttcaaaccagcaactcaaggtgcaattagtggtattaaaatggtggaagcacttcataaagcagaccttccaaaaggtcttgtaaacgtagcgacaggacgtggttctgtaattggtgactacttagtagagcacgaaggtatcaatatggtatcgttcacaggtggtacaaacacaggtaaacatttagcgaaaaaagcttccatgattccacttgtattagaacttggtggtaaagaccctggtatcgttcgtgaagatgctgacttacaagatgctgcaaaccatatcgtaagcggtgcattctcttactctggtcaacgttgtacagctattaaacgtgttcttgtacacgaaaacgtagcagacgagcttgttagcttagtaaaagagcaagtagctaaactatcagttggatctccagaacaagacagcacaatcgttccattaatcgacgataagtctgctgacttcgttcaaggtttagttgacgacgctgtggaaaaaggtgctacaatcgttatcggtaacaaacgtgagcgtaacctaatttacccaacattaatcgaccacgtaacagaagaaatgaaagttgcttgggaagagccattcggcccactccttccaatcatccgtgtttcttcagatgagcaagcaattgaaattgctaacaaatcagagttcggtctgcaagcaagtgtcttcacaaaagacattaacaaagcatttgcaattgctaacaaaatcgaaactggttctgttcaaattaacggacgcacagagcgcggccctgaccacttcccattcatcggtgtaaaaggttcaggtatgggcgcacaaggtattcgtaagagccttgagtctatgacacgtgaaaaagtaactgtattaaacttagtttaa

SEQ ID NO: 42 - protein, 479 aa, Bacillus cereus
MTTSNTYKFYLNGEWRESSSGETIEIPSPYLHEVIGQVQAITRGEVDEAIASAKEAQKSWAEASLQDRAKYLYKWADELVNMQDEIADIIMKEVGKGYKDAKKEVVRTADFIRYTIEEALHMHGESMMGDSFPGGTKSKLAIIQRAPLGVVLAIAPFNYPVNLSAAKLAPALIMGNAVIFKPATQGAISGIKMVEALHKADLPKGLVNVATGRGSVIGDYLVEHEGINMVSFTGGTNTGKHLAKKASMIPLVLELGGKDPGIVREDADLQDAANHIVSGAFSYSGQRCTAIKRVLVHENVADELVSLVKEQVAKLSVGSPEQDSTIVPLIDDKSADFVQGLVDDAVEKGATIVIGNKRERNLIYPTLIDHVTEEMKVAWEEPFGPLLPIIRVSSDEQAIEIANKSEFGLQASVFTKDINKAFAIANKIETGSVQINGRTERGPDHFPFIGVKGSGMGAQGIRKSLESMTREKVTVLNLV

SEQ ID NO: 43 - DNA, 1491 bp, Pisum sativum
  source 1..1491: mol_type = genomic DNA
atggctgctaccggagttctggcagagattatagatggagatgtatacaaatattatgcagatggtgaatggaagaaatccacctctggtaaaagtgttgccatcatcaaccctactacaagaaaacctcaatacaaagttcaagcttgttcacaagaagaggttaataaggttatggattcagcaaaatcagcgcaaaaatcatgggcgaaaactccactttggaagagagctgaacttcttcacaaggctgcagcaattctgaaagaacacaaagctgcaattgcagaatgtttagtgaaagaaatagcaaagccagctaaagatgcagtcactgaggttgtaagatctggtgatttggtttcttactgtgctgaagaaggagtgaggattcttggagagggaaagtttttggtttctgatagttttcctggaaatgaaaggaccaaatattgtctcacttctaagattccattaggagttattttagccattccgccttttaactatcctgtcaatctcgctgtctcgaaaattgctcctgcattgattgctggaaattctattgtgcttaagcctccaactcagggagctgttgcggctctgcacatggttcattgctttcacttggctggttttccgaaaggcctaatcagctgtgttactggaaaaggctcggaaattggtgactttcttacaatgcatccaggagtcaactgtataagcttcactggtggagatactggcattgcgatttcaaagaagtcgggtatgattcctctacaaatggaattgggaggaaaagatgcttgcattgtacttgaagatgctgatcttgatttggttgctgcaaacatcataaaaggaggtttctcatacagtggtcagaggtgtactgctgtgaaggttgttctggtaatggaatcagtagcggatgctttagtcgagaaagtgaaggttaaggtagcgaaactaagtgttggaccgcccgaggatgactctgatatcacaccagttgtatcggaatcatcggccaatttcatcgaaggcttggtgaatgatgcgaaagagaaaggtgcaacattctgtcaggaatacaagagagaagggaatctcatttggcctttgttacttgataatgttaggccagacatgagaattgcatgggaagagccatttggaccggttttgcctgttattaggatcaattcggttgaagaaggtattcatcattgtaatgctagcaacttcggacttcagggatgtgtattcacgaaggatatcaataaagcaataatgattagtgatgcgatggaatcaggaacagttcagataaactccgcaccagctcgtggaccggatcattttccctttcagggaataaaggacagtggaattggatcccagggaattaccaacagcataaacatgatgacaaaagtgaaaactactgtgataaatttgcctagtccttcttataccatgggctaa

SEQ ID NO: 44 - protein, 496 aa, Pisum sativum
MAATGVLAEIIDGDVYKYYADGEWKKSTSGKSVAIINPTTRKPQYKVQACSQEEVNKVMDSAKSAQKSWAKTPLWKRAELLHKAAAILKEHKAAIAECLVKEIAKPAKDAVTEVVRSGDLVSYCAEEGVRILGEGKFLVSDSFPGNERTKYCLTSKIPLGVILAIPPFNYPVNLAVSKIAPALIAGNSIVLKPPTQGAVAALHMVHCFHLAGFPKGLISCVTGKGSEIGDFLTMHPGVNCISFTGGDTGIAISKKSGMIPLQMELGGKDACIVLEDADLDLVAANIIKGGFSYSGQRCTAVKVVLVMESVADALVEKVKVKVAKLSVGPPEDDSDITPVVSESSANFIEGLVNDAKEKGATFCQEYKREGNLIWPLLLDNVRPDMRIAWEEPFGPVLPVIRINSVEEGIHHCNASNFGLQGCVFTKDINKAIMISDAMESGTVQINSAPARGPDHFPFQGIKDSGIGSQGITNSINMMTKVKTTVINLPSPSYTMG

SEQ ID NO: 45 - DNA, 1020 bp, Escherichia coli
  source 1..1020: mol_type = genomic DNA
atgaccgtacgcgtagcgataaatggcttcggtcgcatcgggcgtaatgtggttcgtgctttgtatgaatccggacgccgggcggaaattaccgtggtggcaatcaacgaactggcggatgctgcgggcatggcgcatttgttgaaatatgacaccagccatggccgttttgcatgggaagtacgacaggaacgcgatcaactttttgttggtgatgacgccatccgcgtattgcatgaacgttcactgcaatcgctcccctggcgtgaacttggcgttgatgtagtcctcgactgcaccggcgtatatggctcccgcgagcatggcgaagcgcatattgccgccggggccaaaaaagtgctcttttcacatcctggcagtaacgatctcgacgcgaccgttgtttacggcgtcaatcaggatcaacttcgtgcggaacaccgcatcgtttctaacgcttcctgtaccacgaattgcataattcccgtcatcaaattgttagatgatgcgtacggtattgagtccggcactgtgaccacaattcactccgccatgcacgatcaacaggttattgatgcataccatcctgacctgcgtcgcacccgggcagccagccagtcgatcattccggtcgatactaaactggccgccggtatcacacgattttttccgcaatttaacgatcgctttgaagcgattgcggtacgtgtgccaaccataaatgtgacggcaatcgatttaagcgtgacggtgaagaaacctgtaaaagccaatgaagtcaacctgttgctgcaaaaagcagcacaaggtgcatttcatggtatagttgactatacggaattgccgttggtctctgtagattttaaccacgatccgcacagtgccattgtcgatggcacccaaacccgggtcagtggcgcacacctgatcaaaacgttggtctggtgcgataacgaatggggctttgctaaccgaatgctcgacacgacgttagctatggctactgttgctttcaggtaa

SEQ ID NO: 46 - protein, 339 aa, Escherichia coli
MTVRVAINGFGRIGRNVVRALYESGRRAEITVVAINELADAAGMAHLLKYDTSHGRFAWEVRQERDQLFVGDDAIRVLHERSLQSLPWRELGVDVVLDCTGVYGSREHGEAHIAAGAKKVLFSHPGSNDLDATVVYGVNQDQLRAEHRIVSNASCTTNCIIPVIKLLDDAYGIESGTVTTIHSAMHDQQVIDAYHPDLRRTRAASQSIIPVDTKLAAGITRFFPQFNDRFEAIAVRVPTINVTAIDLSVTVKKPVKANEVNLLLQKAAQGAFHGIVDYTELPLVSVDFNHDPHSAIVDGTQTRVSGAHLIKTLVWCDNEWGFANRMLDTTLAMATVAFR

SEQ ID NO: 47 - DNA, 1001 bp, Escherichia coli
  source 1..1001: mol_type = genomic DNA
atgagtaaagttggtattaacggttttggtcgtatcggtcgactggtgttgggtcgattacttgaagtcaaaagcaacatagacgttgtcgctattaatgatctcacttccccataaattctcgcctacctgctgaaacatgattcaaactacggaccattcccctggagcgttgattttacggaagattcacttatcgttgatgggaaaagtatcgcggtttacgccgaaaaagaggctaaaaatattccgtggaaagcgaaaggtgcagaaatcattgtcgaatgtactggcttttatacctccgccgagaaatcgcaggcgcatcttgatgctggtgcgaagaaggtgttgatttccgcccctgccggtgaaatgaaaactatcgtttataacgtcaatgacgacactctggatggcaacgacaccattgtttccgtggcgtcatgcaccactaactgtcttgcgccgatggccaaagccttgcatgacagtttcgggatagaagtcggcacgatgacgaccattcatgcctatactggcacccagtcactggtggatggcccgcgtggtaaagatttacgtgcttcacgcgcagcggcagaaaatatcattccccacactacgggggcggcaaaagccattggtctggtgatcccggaactgagcggcaaactgaaaggtcatgcgcaacgcgtgccggtgaaaacaggttcggtcactgaactggtatcgattctcggaaaaaagtgactgccgaagaggtgaataacgcacttaaacaagcaaccaccaataacgagtcatttggttataccgatgaagaaatagtctcttccgatatcattggcagccatttcggttcggtgtttgatgccacgcaaacggaaattaccgccgtgggcgatttacaactggtgaaaacggtcgcctggtacgataacgaatatggcttcgtcacacagcttattcgcaccctcgaaaaattcgctaaactctga

SEQ ID NO: 48 - DNA, 1401 bp, Escherichia coli
  source 1..1401: mol_type = genomic DNA
atgccacattcctacgattacgatgccatagtaataggttccggccccggcggcgaaggcgctgcaatgggcctggttaagcaaggtgcgcgcgtcgcagttatcgagcgttatcaaaatgttggcggcggttgcacccactggggcaccatcccgtcgaaagctctccgtcacgccgtcagccgcattatagaattcaatcaaaacccactttacagcgaccattcccgactgctccgctcttcttttgccgatatccttaaccatgccgataacgtgattaatcaacaaacgcgcatgcgtcagggattttacgaacgtaatcactgtgaaatattgcagggaaacgctcgctttgttgacgagcatacgttggcgctggattgcccggacggcagcgttgaaacactaaccgctgaaaaatttgttattgcctgcggctctcgtccatatcatccaacagatgttgatttcacccatccacgcatttacgacagcgactcaattctcagcatgcaccacgaaccgcgccatgtacttatctatggtgctggagtgatcggctgtgaatatgcgtcgatcttccgcggtatggatgtaaaagtggatctgatcaacacccgcgatcgcctgctggcatttctcgatcaagagatgtcagattctctctcctatcacttctggaacagtggcgtagtgattcgtcacaacgaagagtacgagaagatcgaaggctgtgacgatggtgtgatcatgcatctgaagtcgggtaaaaaactgaaagctgactgcctgctctatgccaacggtcgcaccggtaataccgattcgctggcgttacagaacattgggctagaaactgacagccgcggacagctgaaggtcaacagcatgtatcagaccgcacagccacacgtttacgcggtgggcgacgtgattggttatccgagcctggcgtcggcggcctatgaccaggggcgcattgccgcgcaggcgctggtaaaaggcgaagccaccgcacatctgattgaagatatccctaccggtatttacaccatcccggaaatcagctctgtgggcaaaaccgaacagcagctgaccgcaatgaaagtgccatatgaagtgggccgcgcccagtttaaacatctggcacgcgcacaaatcgtcggcatgaacgtgggcacgctgaaaattttgttccatcgggaaacaaaagagattctgggtattcactgctttggcgagcgcgctgccgaaattattcatatcggtcaggcgattatggaacagaaaggtggcggcaacactattgagtacttcgtcaacaccacctttaactacccgacgatggcggaagcctatcgggtagctgcgttaaacggtttaaaccgcctgttttaa

SEQ ID NO: 49 - protein, 466 aa, Escherichia coli
MPHSYDYDAIVIGSGPGGEGAAMGLVKQGARVAVIERYQNVGGGCTHWGTIPSKALRHAVSRIIEFNQNPLYSDHSRLLRSSFADILNHADNVINQQTRMRQGFYERNHCEILQGNARFVDEHTLALDCPDGSVETLTAEKFVIACGSRPYHPTDVDFTHPRIYDSDSILSMHHEPRHVLIYGAGVIGCEYASIFRGMDVKVDLINTRDRLLAFLDQEMSDSLSYHFWNSGVVIRHNEEYEKIEGCDDGVIMHLKSGKKLKADCLLYANGRTGNTDSLALQNIGLETDSRGQLKVNSMYQTAQPHVYAVGDVIGYPSLASAAYDQGRIAAQALVKGEATAHLIEDIPTGIYTIPEISSVGKTEQQLTAMKVPYEVGRAQFKHLARAQIVGMNVGTLKILFHRETKEILGIHCFGERAAEIIHIGQAIMEQKGGGNTIEYFVNTTFNYPTMAEAYRVAALNGLNRLF

SEQ ID NO: 50 - DNA, 2664 bp, Escherichia coli
  source 1..2664: mol_type = genomic DNA
atgtcagaacgtttcccaaatgacgtggatccgatcgaaactcgcgactggctccaggcgatcgaatcggtcatccgtgaagaaggtgttgagcgtgctcagtatctgatcgaccaactgcttgctgaagcccgcaaaggcggtgtaaacgtagccgcaggcacaggtatcagcaactacatcaacaccatccccgttgaagaacaaccggagtatccgggtaatctggaactggaacgccgtattcgttcagctatccgctggaacgccatcatgacggtgctgcgtgcgtcgaaaaaagacctcgaactgggcggccatatggcgtccttccagtcttccgcaaccatttatgatgtgtgctttaaccacttcttccgtgcacgcaacgagcaggatggcggcgacctggtttacttccagggccacatctccccgggcgtgtacgctcgtgctttcctggaaggtcgtctgactcaggagcagctggataacttccgtcaggaagttcacggcaatggcctctcttcctatccgcacccgaaactgatgccggaattctggcagttcccgaccgtatctatgggtctgggtccgattggtgctatttaccaggctaaattcctgaaatatctggaacaccgtggcctgaaagatacctctaaacaaaccgtttacgcgttcctcggtgacggtgaaatggacgaaccggaatccaaaggtgcgatcaccatcgctacccgtgaaaaactggataacctggtcttcgttatcaactgtaacctgcagcgtcttgacggcccggtcaccggtaacggcaagatcatcaacgaactggaaggcatcttcgaaggtgctggctggaacgtgatcaaagtgatgtggggtagccgttgggatgaactgctgcgtaaggataccagcggtaaactgatccagctgatgaacgaaaccgttgacggcgactaccagaccttcaaatcgaaagatggtgcgtacgttcgtgaacacttcttcggtaaatatcctgaaaccgcagcactggttgcagactggactgacgagcagatctgggcactgaaccgtggtggtcacgatccgaagaaaatctacgctgcattcaagaaagcgcaggaaaccaaaggcaaagcgacagtaatccttgctcataccattaaaggttacggcatgggcgacgcggctgaaggtaaaaacatcgcgcaccaggttaagaaaatgaacatggacggtgtgcgtcatatccgcgaccgtttcaatgtgccggtgtctgatgcagatatcgaaaaactgccgtacatcaccttcccggaaggttctgaagagcatacctatctgcacgctcagcgtcagaaactgcacggttatctgccaagccgtcagccgaacttcaccgagaagcttgagctgccgagcctgcaagacttcggcgcgctgttggaagagcagagcaaagagatctctaccactatcgctttcgttcgtgctctgaacgtgatgctgaagaacaagtcgatcaaagatcgtctggtaccgatcatcgccgacgaagcgcgtactttcggtatggaaggtctgttccgtcagattggtatttacagcccgaacggtcagcagtacaccccgcaggaccgcgagcaggttgcttactataaagaagacgagaaaggtcagattctgcaggaagggatcaacgagctgggcgcaggttgttcctggctggcagcggcgacctcttacagcaccaacaatctgccgatgatcccgttctacatctattactcgatgttcggcttccagcgtattggcgatctgtgctgggcggctggcgaccagcaagcgcgtggcttcctgatcggcggtacttccggtcgtaccaccctgaacggcgaaggtctgcagcacgaagatggtcacagccacattcagtcgctgactatcccgaactgtatctcttacgacccggcttacgcttacgaagttgctgtcatcatgcatgacggtctggagcgtatgtacggtgaaaaacaagagaacgtttactactacatcactacgctgaacgaaaactaccacatgccggcaatgccggaaggtgctgaggaaggtatccgtaaaggtatctacaaactcgaaactattgaaggtagcaaaggtaaagttcagctgctcggctccggttctatcctgcgtcacgtccgtgaagcagctgagatcctggcgaaagattacggcgtaggttctgacgtttatagcgtgacctccttcaccgagctggcgcgtgatggtcaggattgtgaacgctggaacatgctgcacccgctggaaactccgcgcgttccgtatatcgctcaggtgatgaacgacgctccggcagtggcatctaccgactatatgaaactgttcgctgagcaggtccgtacttacgtaccggctgacgactaccgcgtactgggtactgatggcttcggtcgttccgacagccgtgagaacctgcgtcaccacttcgaagttgatgcttcttatgtcgtggttgcggcgctgggcgaactggctaaacgtggcgaaatcgataagaaagtggttgctgacgcaatcgccaaattcaacatcgatgcagataaagttaacccgcgtctggcgtaa

SEQ ID NO: 51 - protein, 887 aa, Escherichia coli
MSERFPNDVDPIETRDWLQAIESVIREEGVERAQYLIDQLLAEARKGGVNVAAGTGISNYINTIPVEEQPEYPGNLELERRIRSAIRWNAIMTVLRASKKDLELGGHMASFQSSATIYDVCFNHFFRARNEQDGGDLVYFQGHISPGVYARAFLEGRLTQEQLDNFRQEVHGNGLSSYPHPKLMPEFWQFPTVSMGLGPIGAIYQAKFLKYLEHRGLKDTSKQTVYAFLGDGEMDEPESKGAITIATREKLDNLVFVINCNLQRLDGPVTGNGKIINELEGIFEGAGWNVIKVMWGSRWDELLRKDTSGKLIQLMNETVDGDYQTFKSKDGAYVREHFFGKYPETAALVADWTDEQIWALNRGGHDPKKIYAAFKKAQETKGKATVILAHTIKGYGMGDAAEGKNIAHQVKKMNMDGVRHIRDRFNVPVSDADIEKLPYITFPEGSEEHTYLHAQRQKLHGYLPSRQPNFTEKLELPSLQDFGALLEEQSKEISTTIAFVRALNVMLKNKSIKDRLVPIIADEARTFGMEGLFRQIGIYSPNGQQYTPQDREQVAYYKEDEKGQILQEGINELGAGCSWLAAATSYSTNNLPMIPFYIYYSMFGFQRIGDLCWAAGDQQARGFLIGGTSGRTTLNGEGLQHEDGHSHIQSLTIPNCISYDPAYAYEVAVIMHDGLERMYGEKQENVYYYITTLNENYHMPAMPEGAEEGIRKGIYKLETIEGSKGKVQLLGSGSILRHVREAAEILAKDYGVGSDVYSVTSFTELARDGQDCERWNMLHPLETPRVPYIAQVMNDAPAVASTDYMKLFAEQVRTYVPADDYRVLGTDGFGRSDSRENLRHHFEVDASYVVVAALGELAKRGEIDKKVVADAIAKFNIDADKVNPRLA

SEQ ID NO: 52 - DNA, 1893 bp, Escherichia coli
  source 1..1893: mol_type = genomic DNA
atggctatcgaaatcaaagtaccggacatcggggctgatgaagttgaaatcaccgagatcctggtcaaagtgggcgacaaagttgaagccgaacagtcgctgatcaccgtagaaggcgacaaagcctctatggaagttccgtctccgcaggcgggtatcgttaaagagatcaaagtctctgttggcgataaaacccagaccggcgcactgattatgattttcgattccgccgacggtgcagcagacgctgcacctgctcaggcagaagagaagaaagaagcagctccggcagcagcaccagcggctgcggcggcaaaagacgttaacgttccggatatcggcagcgacgaagttgaagtgaccgaaatcctggtgaaagttggcgataaagttgaagctgaacagtcgctgatcaccgtagaaggcgacaaggcttctatggaagttccggctccgtttgctggcaccgtgaaagagatcaaagtgaacgtgggtgacaaagtgtctaccggctcgctgattatggtcttcgaagtcgcgggtgaagcaggcgcggcagctccggccgctaaacaggaagcagctccggcagcggcccctgcaccagcggctggcgtgaaagaagttaacgttccggatatcggcggtgacgaagttgaagtgactgaagtgatggtgaaagtgggcgacaaagttgccgctgaacagtcactgatcaccgtagaaggcgacaaagcttctatggaagttccggcgccgtttgcaggcgtcgtgaaggaactgaaagtcaacgttggcgataaagtgaaaactggctcgctgattatgatcttcgaagttgaaggcgcagcgcctgcggcagctcctgcgaaacaggaagcggcagcgccggcaccggcagcaaaagctgaagccccggcagcagcaccagctgcgaaagcggaaggcaaatctgaatttgctgaaaacgacgcttatgttcacgcgactccgctgatccgccgtctggcacgcgagtttggtgttaaccttgcgaaagtgaagggcactggccgtaaaggtcgtatcctgcgcgaagacgttcaggcttacgtgaaagaagctatcaaacgtgcagaagcagctccggcagcgactggcggtggtatccctggcatgctgccgtggccgaaggtggacttcagcaagtttggtgaaatcgaagaagtggaactgggccgcatccagaaaatctctggtgcgaacctgagccgtaactgggtaatgatcccgcatgttactcacttcgacaaaaccgatatcaccgagttggaagcgttccgtaaacagcagaacgaagaagcggcgaaacgtaagctggatgtgaagatcaccccggttgtcttcatcatgaaagccgttgctgcagctcttgagcagatgcctcgcttcaatagttcgctgtcggaagacggtcagcgtctgaccctgaagaaatacatcaacatcggtgtggcggtggataccccgaacggtctggttgttccggtattcaaagacgtcaacaagaaaggcatcatcgagctgtctcgcgagctgatgactatttctaagaaagcgcgtgacggtaagctgactgcgggcgaaatgcagggcggttgcttcaccatctccagcatcggcggcctgggtactacccacttcgcgccgattgtgaacgcgccggaagtggctatcctcggcgtttccaagtccgcgatggagccggtgtggaatggtaaagagttcgtgccgcgtctgatgctgccgatttctctctccttcgaccaccgcgtgatcgacggtgctgatggtgcccgtttcattaccatcattaacaacacgctgtctgacattcgccgtctggtgatgtaa

SEQ ID NO: 53 - protein, 630 aa, Escherichia coli
MAIEIKVPDIGADEVEITEILVKVGDKVEAEQSLITVEGDKASMEVPSPQAGIVKEIKVSVGDKTQTGALIMIFDSADGAADAAPAQAEEKKEAAPAAAPAAAAAKDVNVPDIGSDEVEVTEILVKVGDKVEAEQSLITVEGDKASMEVPAPFAGTVKEIKVNVGDKVSTGSLIMVFEVAGEAGAAAPAAKQEAAPAAAPAPAAGVKEVNVPDIGGDEVEVTEVMVKVGDKVAAEQSLITVEGDKASMEVPAPFAGVVKELKVNVGDKVKTGSLIMIFEVEGAAPAAAPAKQEAAAPAPAAKAEAPAAAPAAKAEGKSEFAENDAYVHATPLIRRLAREFGVNLAKVKGTGRKGRILREDVQAYVKEAIKRAEAAPAATGGGIPGMLPWPKVDFSKFGEIEEVELGRIQKISGANLSRNWVMIPHVTHFDKTDITELEAFRKQQNEEAAKRKLDVKITPVVFIMKAVAAALEQMPRFNSSLSEDGQRLTLKKYINIGVAVDTPNGLVVPVFKDVNKKGIIELSRELMTISKKARDGKLTAGEMQGGCFTISSIGGLGTTHFAPIVNAPEVAILGVSKSAMEPVWNGKEFVPRLMLPISLSFDHRVIDGADGARFITIINNTLSDIRRLVM

SEQ ID NO: 54 - DNA, 1719 bp, Escherichia coli
  source 1..1719: mol_type = genomic DNA
atgaaacaaacggttgcagcttatatcgccaaaacactcgaatcggcaggggtgaaacgcatctggggagtcacaggcgactctctgaacggtcttagtgacagtcttaatcgcatgggcaccatcgagtggatgtccacccgccacgaagaagtggcggcctttgccgctggcgctgaagcacaacttagcggagaactggcggtctgcgccggatcgtgcggccccggcaacctgcacttaatcaacggcctgttcgattgccaccgcaatcacgttccggtactggcgattgccgctcatattccctccagcgaaattggcagcggctatttccaggaaacccacccacaagagctattccgcgaatgtagtcactattgcgagctggtttccagcccggagcagatcccacaagtactggcgattgccatgcgcaaagcggtgcttaaccgtggcgtttcggttgtcgtgttaccaggcgacgtggcgttaaaacctgcgccagaaggggcaaccatgcactggtatcatgcgccacaaccagtcgtgacgccggaagaagaagagttacgcaaactggcgcaactgctgcgttattccagcaatatcgccctgatgtgtggcagcggctgcgcgggggcgcataaagagttagttgagtttgccgggaaaattaaagcgcctattgttcatgccctgcgcggtaaagaacatgtcgaatacgataatccgtatgatgttggaatgaccgggttaatcggcttctcgtcaggtttccataccatgatgaacgccgacacgttagtgctactcggcacgcaatttccctaccgcgccttctacccgaccgatgccaaaatcattcagattgatatcaacccagccagcatcggcgctcacagcaaggtggatatggcactggtcggcgatatcaagtcgactctgcgtgcattgcttccattggtggaagaaaaagccgatcgcaagtttctggataaagcgctggaagattaccgcgacgcccgcaaagggctggacgatttagctaaaccgagcgagaaagccattcacccgcaatatctggcgcagcaaattagtcattttgccgccgatgacgctattttcacctgtgacgttggtacgccaacggtgtgggcggcacgttatctaaaaatgaacggcaagcgtcgcctgttaggttcgtttaaccacggttcgatggctaacgccatgccgcaggcgctgggtgcgcaggcgacagagccagaacgtcaggtggtcgccatgtgcggcgatggcggttttagcatgttgatgggcgatttcctctcagtagtgcagatgaaactgccagtgaaaattgtcgtctttaacaacagcgtgctgggctttgtggcgatggagatgaaagctggtggctatttgactgacggcaccgaactacacgacacaaactttgcccgcattgccgaagcgtgcggcattacgggtatccgtgtagaaaaagcgtctgaagttgatgaagccctgcaacgcgccttctccatcgacggtccggtgttggtggatgtggtggtcgccaaagaagagttagccattccaccgcagatcaaactcgaacaggccaaaggtttcagcctgtatatgctgcgcgcaatcatcagcggacgcggtgatgaagtgatcgaactggcgaaaacaaactggctaaggtaa

SEQ ID NO: 55 - protein, 572 aa, Escherichia coli
MKQTVAAYIAKTLESAGVKRIWGVTGDSLNGLSDSLNRMGTIEWMSTRHEEVAAFAAGAEAQLSGELAVCAGSCGPGNLHLINGLFDCHRNHVPVLAIAAHIPSSEIGSGYFQETHPQELFRECSHYCELVSSPEQIPQVLAIAMRKAVLNRGVSVVVLPGDVALKPAPEGATMHWYHAPQPVVTPEEEELRKLAQLLRYSSNIALMCGSGCAGAHKELVEFAGKIKAPIVHALRGKEHVEYDNPYDVGMTGLIGFSSGFHTMMNADTLVLLGTQFPYRAFYPTDAKIIQIDINPASIGAHSKVDMALVGDIKSTLRALLPLVEEKADRKFLDKALEDYRDARKGLDDLAKPSEKAIHPQYLAQQISHFAADDAIFTCDVGTPTVWAARYLKMNGKRRLLGSFNHGSMANAMPQALGAQATEPERQVVAMCGDGGFSMLMGDFLSVVQMKLPVKIVVFNNSVLGFVAMEMKAGGYLTDGTELHDTNFARIAEACGITGIRVEKASEVDEALQRAFSIDGPVLVDVVVAKEELAIPPQIKLEQAKGFSLYMLRAIISGRGDEVIELAKTNWLR

SEQ ID NO: 56 - DNA, 2802 bp, Escherichia coli
  source 1..2802: mol_type = genomic DNA
atgcagaacagcgctttgaaagcctggttggactcttcttacctctctggcgcaaaccagagctggatagaacagctctatgaagacttcttaaccgatcctgactcggttgacgctaactggcgttcgacgttccagcagttacctggtacgggagtcaaaccggatcaattccactctcaaacgcgtgaatatttccgccgcctggcgaaagacgcttcacgttactcttcaacgatctccgaccctgacaccaatgtgaagcaggttaaagtcctgcagctcattaacgcataccgcttccgtggtcaccagcatgcgaatctcgatccgctgggactgtggcagcaagataaagtggccgatctggatccgtctttccacgatctgaccgaagcagacttccaggagaccttcaacgtcggttcatttgccagcggcaaagaaaccatgaaactcggcgagctgctggaagccctcaagcaaacctactgcggcccgattggtgccgagtatatgcacattaccagcaccgaagaaaaacgctggatccaacagcgtatcgagtctggtcgcgcgactttcaatagcgaagagaaaaaacgcttcttaagcgaactgaccgccgctgaaggtcttgaacgttacctcggcgcaaaattccctggcgcaaaacgcttctcgctggaaggcggtgacgcgttaatcccgatgcttaaagagatgatccgccacgctggcaacagcggcacccgcgaagtggttctcgggatggcgcaccgtggtcgtctgaacgtgctggtgaacgtgctgggtaaaaaaccgcaagacttgttcgacgagttcgccggtaaacataaagaacacctcggcacgggtgacgtgaaataccacatgggcttctcgtctgacttccagaccgatggcggcctggtgcacctggcgctggcgtttaacccgtctcaccttgagattgtaagcccggtagttatcggttctgttcgtgcccgtctggacagacttgatgagccgagcagcaacaaagtgctgccaatcaccatccacggtgacgccgcagtgaccgggcagggcgtggttcaggaaaccctgaacatgtcgaaagcgcgtggttatgaagttggcggtacggtacgtatcgttatcaacaaccaggttggtttcaccacctctaatccgctggatgcccgttctacgccgtactgtactgatatcggtaagatggttcaggccccgattttccacgttaacgcggacgatccggaagccgttgcctttgtgacccgtctggcgctcgatttccgtaacacctttaaacgtgatgtcttcatcgacctggtgtgctaccgccgtcacggccacaacgaagccgacgagccgagcgcaacccagccgctgatgtatcagaaaatcaaaaaacatccgacaccgcgcaaaatctacgctgacaagctggagcaggaaaaagtggcgacgctggaagatgccaccgagatggttaacctgtaccgcgatgcgctggatgctggcgattgcgtagtggcagagtggcgtccgatgaacatgcactctttcacctggtcgccgtacctcaaccacgaatgggacgaagagtacccgaacaaagttgagatgaagcgcctgcaggagctggcgaaacgcatcagcacggtgccggaagcagttgaaatgcagtctcgcgttgccaagatttatggcgatcgccaggcgatggctgccggtgagaaactgttcgactggggcggtgcggaaaacctcgcttacgccacgctggttgatgaaggcattccggttcgcctgtcgggtgaagactccggtcgcggtaccttcttccaccgccacgcggtgatccacaaccagtctaacggttccacttacacgccgctgcaacatatccataacgggcagggcgcgttccgtgtctgggactccgtactgtctgaagaagcagtgctggcgtttgaatatggttatgccaccgcagaaccacgcactctgaccatctgggaagcgcagttcggtgacttcgccaacggtgcgcaggtggttatcgaccagttcatctcctctggcgaacagaaatggggccggatgtgtggtctggtgatgttgctgccgcacggttacgaagggcaggggccggagcactcctccgcgcgtctggaacgttatctgcaactttgtgctgagcaaaacatgcaggtttgcgtaccgtctaccccggcacaggtttaccacatgctgcgtcgtcaggcgctgcgcgggatgcgtcgtccgctggtcgtgatgtcgccgaaatccctgctgcgtcatccgctggcggtttccagcctcgaagaactggcgaacggcaccttcctgccagccatcggtgaaatcgacgagcttgatccgaagggcgtgaagcgcgtagtgatgtgttctggtaaggtttattacgacctgctggaacagcgtcgtaagaacaatcaacacgatgtcgccattgtgcgtatcgagcaactctacccgttcccgcataaagcgatgcaggaagtgttgcagcagtttgctcacgtcaaggattttgtctggtgccaggaagagccgctcaaccagggcgcatggtactgcagccagcatcatttccgtgaagtgattccgtttggggcttctctgcgttatgcaggccgcccggcctccgcctctccggcggtagggtatatgtccgttcaccagaaacagcaacaagatctggttaatgacgcgctgaacgtcgaataa

SEQ ID NO: 57 - protein, 933 aa, Escherichia coli
MQNSALKAWLDSSYLSGANQSWIEQLYEDFLTDPDSVDANWRSTFQQLPGTGVKPDQFHSQTREYFRRLAKDASRYSSTISDPDTNVKQVKVLQLINAYRFRGHQHANLDPLGLWQQDKVADLDPSFHDLTEADFQETFNVGSFASGKETMKLGELLEALKQTYCGPIGAEYMHITSTEEKRWIQQRIESGRATFNSEEKKRFLSELTAAEGLERYLGAKFPGAKRFSLEGGDALIPMLKEMIRHAGNSGTREVVLGMAHRGRLNVLVNVLGKKPQDLFDEFAGKHKEHLGTGDVKYHMGFSSDFQTDGGLVHLALAFNPSHLEIVSPVVIGSVRARLDRLDEPSSNKVLPITIHGDAAVTGQGVVQETLNMSKARGYEVGGTVRIVINNQVGFTTSNPLDARSTPYCTDIGKMVQAPIFHVNADDPEAVAFVTRLALDFRNTFKRDVFIDLVCYRRHGHNEADEPSATQPLMYQKIKKHPTPRKIYADKLEQEKVATLEDATEMVNLYRDALDAGDCVVAEWRPMNMHSFTWSPYLNHEWDEEYPNKVEMKRLQELAKRISTVPEAVEMQSRVAKIYGDRQAMAAGEKLFDWGGAENLAYATLVDEGIPVRLSGEDSGRGTFFHRHAVIHNQSNGSTYTPLQHIHNGQGAFRVWDSVLSEEAVLAFEYGYATAEPRTLTIWEAQFGDFANGAQVVIDQFISSGEQKWGRMCGLVMLLPHGYEGQGPEHSSARLERYLQLCAEQNMQVCVPSTPAQVYHMLRRQALRGMRRPLVVMSPKSLLRHPLAVSSLEELANGTFLPAIGEIDELDPKGVKRVVMCSGKVYYDLLEQRRKNNQHDVAIVRIEQLYPFPHKAMQEVLQQFAHVKDFVWCQEEPLNQGAWYCSQHHFREVIPFGASLRYAGRPASASPAVGYMSVHQKQQQDLVNDALNVE

SEQ ID NO: 58 - DNA, 1218 bp, Escherichia coli
  source 1..1218: mol_type = genomic DNA
atgagtagcgtagatattctggtccctgacctgcctgaatccgtagccgatgccaccgtcgcaacctggcataaaaaacccggcgacgcagtcgtacgtgatgaagtgctggtagaaatcgaaactgacaaagtggtactggaagtaccggcatcagcagacggcattctggatgcggttctggaagatgaaggtacaacggtaacgtctcgtcagatccttggtcgcctgcgtgaaggcaacagcgccggtaaagaaaccagcgccaaatctgaagagaaagcgtccactccggcgcaacgccagcaggcgtctctggaagagcaaaacaacgatgcgttaagcccggcgatccgtcgcctgctggctgaacacaatctcgacgccagcgccattaaaggcaccggtgtgggtggtcgtctgactcgtgaagatgtggaaaaacatctggcgaaagccccggcgaaagagtctgctccggcagcggctgctccggcggcgcaaccggctctggctgcacgtagtgaaaaacgtgtcccgatgactcgcctgcgtaagcgtgtggcagagcgtctgctggaagcgaaaaactccaccgccatgctgaccacgttcaacgaagtcaacatgaagccgattatggatctgcgtaagcagtacggtgaagcgtttgaaaaacgccacggcatccgtctgggctttatgtccttctacgtgaaagcggtggttgaagccctgaaacgttacccggaagtgaacgcttctatcgacggcgatgacgtggtttaccacaactatttcgacgtcagcatggcggtttctacgccgcgcggcctggtgacgccggttctgcgtgatgtcgataccctcggcatggcagacatcgagaagaaaatcaaagagctggcagtcaaaggccgtgacggcaagctgaccgttgaagatctgaccggtggtaacttcaccatcaccaacggtggtgtgttcggttccctgatgtctacgccgatcatcaacccgccgcagagcgcaattctgggtatgcacgctatcaaagatcgtccgatggcggtgaatggtcaggttgagatcctgccgatgatgtacctggcgctgtcctacgatcaccgtctgatcgatggtcgcgaatccgtgggcttcctggtaacgatcaaagagttgctggaagatccgacgcgtctgctgctggacgtgtag

SEQ ID NO: 59 - protein, 405 aa, Escherichia coli
MSSVDILVPDLPESVADATVATWHKKPGDAVVRDEVLVEIETDKVVLEVPASADGILDAVLEDEGTTVTSRQILGRLREGNSAGKETSAKSEEKASTPAQRQQASLEEQNNDALSPAIRRLLAEHNLDASAIKGTGVGGRLTREDVEKHLAKAPAKESAPAAAAPAAQPALAARSEKRVPMTRLRKRVAERLLEAKNSTAMLTTFNEVNMKPIMDLRKQYGEAFEKRHGIRLGFMSFYVKAVVEALKRYPEVNASIDGDDVVYHNYFDVSMAVSTPRGLVTPVLRDVDTLGMADIEKKIKELAVKGRDGKLTVEDLTGGNFTITNGGVFGSLMSTPIINPPQSAILGMHAIKDRPMAVNGQVEILPMMYLALSYDHRLIDGRESVGFLVTIKELLEDPTRLLLDV

SEQ ID NO: 60 - DNA, 2145 bp, Escherichia coli
  source 1..2145: mol_type = genomic DNA
gtgtcccgtattattatgctgatccctaccggaaccagcgtcggtctgaccagcgtcagccttggcgtgatccgtgcaatggaacgcaaaggcgttcgtctgagcgttttcaaacctatcgctcagccgcgtaccggtggcgatgcgcccgatcagactacgactatcgtgcgtgcgaactcttccaccacgacggccgctgaaccgctgaaaatgagctacgttgaaggtctgctttccagcaatcagaaagatgtgctgatggaagagatcgtcgcaaactaccacgctaacaccaaagacgctgaagtcgttctggttgaaggtctggtcccgacacgtaagcaccagtttgcccagtctctgaactacgaaatcgctaaaacgctgaatgcggaaatcgtcttcgttatgtctcagggcactgacaccccggaacagctgaaagagcgtatcgaactgacccgcaacagcttcggcggtgccaaaaacaccaacatcaccggcgttatcgttaacaaactgaacgcaccggttgatgaacagggtcgtactcgcccggatctgtccgagattttcgacgactcttccaaagctaaagtaaacaatgttgatccggcgaagctgcaagaatccagcccgctgccggttctcggcgctgtgccgtggagctttgacctgatcgcgactcgtgcgatcgatatggctcgccacctgaatgcgaccatcatcaacgaaggcgacatcaatactcgccgcgttaaatccgtcactttctgcgcacgcagcattccgcacatgctggagcacttccgtgccggttctctgctggtgacttccgcagaccgtcctgacgtgctggtggccgcttgcctggcagccatgaacggcgtagaaatcggtgccctgctgctgactggcggttacgaaatggacgcgcgcatttctaaactgtgcgaacgtgctttcgctaccggcctgccggtatttatggtgaacaccaacacctggcagacctctctgagcctgcagagcttcaacctggaagttccggttgacgatcacgaacgtatcgagaaagttcaggaatacgttgctaactacatcaacgctgactggatcgaatctctgactgccacttctgagcgcagccgtcgtctgtctccgcctgcgttccgttatcagctgactgaacttgcgcgcaaagcgggcaaacgtatcgtactgccggaaggtgacgaaccgcgtaccgttaaagcagccgctatctgtgctgaacgtggtatcgcaacttgcgtactgctgggtaatccggcagagatcaaccgtgttgcagcgtctcagggtgtagaactgggtgcagggattgaaatcgttgatccagaagtggttcgcgaaagctatgttggtcgtctggtcgaactgcgtaagaacaaaggcatgaccgaaaccgttgcccgcgaacagctggaagacaacgtggtgctcggtacgctgatgctggaacaggatgaagttgatggtctggtttccggtgctgttcacactaccgcaaacaccatccgtccgccgctgcagctgatcaaaactgcaccgggcagctccctggtatcttccgtgttcttcatgctgctgccggaacaggtttacgtttacggtgactgtgcgatcaacccggatccgaccgctgaacagctggcagaaatcgcgattcagtccgctgattccgctgcggccttcggtatcgaaccgcgcgttgctatgctctcctactccaccggtacttctggtgcaggtagcgacgtagaaaaagttcgcgaagcaactcgtctggcgcaggaaaaacgtcctgacctgatgatcgacggtccgctgcagtacgacgctgcggtaatggctgacgttgcgaaatccaaagcgccgaactctccggttgcaggtcgcgctaccgtgttcatcttcccggatctgaacaccggtaacaccacctacaaagcggtacagcgttctgccgacctgatctccatcgggccgatgctgcagggtatgcgcaagccggttaacgacctgtcccgtggcgcactggttgacgatatcgtctacaccatcgcgctgactgcgattcagtctgcacagcagcagtaa

SEQ ID NO: 61 - protein, 714 aa, Escherichia coli
VSRIIMLIPTGTSVGLTSVSLGVIRAMERKGVRLSVFKPIAQPRTGGDAPDQTTTIVRANSSTTTAAEPLKMSYVEGLLSSNQKDVLMEEIVANYHANTKDAEVVLVEGLVPTRKHQFAQSLNYEIAKTLNAEIVFVMSQGTDTPEQLKERIELTRNSFGGAKNTNITGVIVNKLNAPVDEQGRTRPDLSEIFDDSSKAKVNNVDPAKLQESSPLPVLGAVPWSFDLIATRAIDMARHLNATIINEGDINTRRVKSVTFCARSIPHMLEHFRAGSLLVTSADRPDVLVAACLAAMNGVEIGALLLTGGYEMDARISKLCERAFATGLPVFMVNTNTWQTSLSLQSFNLEVPVDDHERIEKVQEYVANYINADWIESLTATSERSRRLSPPAFRYQLTELARKAGKRIVLPEGDEPRTVKAAAICAERGIATCVLLGNPAEINRVAASQGVELGAGIEIVDPEVVRESYVGRLVELRKNKGMTETVAREQLEDNVVLGTLMLEQDEVDGLVSGAVHTTANTIRPPLQLIKTAPGSSLVSSVFFMLLPEQVYVYGDCAINPDPTAEQLAEIAIQSADSAAAFGIEPRVAMLSYSTGTSGAGSDVEKVREATRLAQEKRPDLMIDGPLQYDAAVMADVAKSKAPNSPVAGRATVFIFPDLNTGNTTYKAVQRSADLISIGPMLQGMRKPVNDLSRGALVDDIVYTIALTAIQSAQQQ

SEQ ID NO: 62 - DNA, 1344 bp, Escherichia coli
  source 1..1344: mol_type = genomic DNA
atggatcagacatattctctggagtcattcctcaaccatgtccaaaagcgcgacccgaatcaaaccgagttcgcgcaagccgttcgtgaagtaatgaccacactctggccttttcttgaacaaaatccaaaatatcgccagatgtcattactggagcgtctggttgaaccggagcgcgtgatccagtttcgcgtggtatgggttgatgatcgcaaccagatacaggtcaaccgtgcatggcgtgtgcagttcagctctgccatcggcccgtacaaaggcggtatgcgcttccatccgtcagttaacctttccattctcaaattcctcggctttgaacaaaccttcaaaaatgccctgactactctgccgatgggcggtggtaaaggcggcagcgatttcgatccgaaaggaaaaagcgaaggtgaagtgatgcgtttttgccaggcgctgatgactgaactgtatcgccacctgggcgcggataccgacgttccggcaggtgatatcggggttggtggtcgtgaagtcggctttatggcggggatgatgaaaaagctctccaacaataccgcctgcgtcttcaccggtaagggcctttcatttggcggcagtcttattcgcccggaagctaccggctacggtctggtttatttcacagaagcaatgctaaaacgccacggtatgggttttgaagggatgcgcgtttccgtttctggctccggcaacgtcgcccagtacgctatcgaaaaagcgatggaatttggtgctcgtgtgatcactgcgtcagactccagcggcactgtagttgatgaaagcggattcacgaaagagaaactggcacgtcttatcgaaatcaaagccagccgcgatggtcgagtggcagattacgccaaagaatttggtctggtctatctcgaaggccaacagccgtggtctctaccggttgatatcgccctgccttgcgccacccagaatgaactggatgttgacgccgcgcatcagcttatcgctaatggcgttaaagccgtcgccgaaggggcaaatatgccgaccaccatcgaagcgactgaactgttccagcaggcaggcgtactatttgcaccgggtaaagcggctaatgctggtggcgtcgctacatcgggcctggaaatggcacaaaacgctgcgcgcctgggctggaaagccgagaaagttgacgcacgtttgcatcacatcatgctggatatccaccatgcctgtgttgagcatggtggtgaaggtgagcaaaccaactacgtgcagggcgcgaacattgccggttttgtgaaggttgccgatgcgatgctggcgcagggtgtgatttaa

SEQ ID NO: 63 - protein, 447 aa, Escherichia coli
MDQTYSLESFLNHVQKRDPNQTEFAQAVREVMTTLWPFLEQNPKYRQMSLLERLVEPERVIQFRVVWVDDRNQIQVNRAWRVQFSSAIGPYKGGMRFHPSVNLSILKFLGFEQTFKNALTTLPMGGGKGGSDFDPKGKSEGEVMRFCQALMTELYRHLGADTDVPAGDIGVGGREVGFMAGMMKKLSNNTACVFTGKGLSFGGSLIRPEATGYGLVYFTEAMLKRHGMGFEGMRVSVSGSGNVAQYAIEKAMEFGARVITASDSSGTVVDESGFTKEKLARLIEIKASRDGRVADYAKEFGLVYLEGQQPWSLPVDIALPCATQNELDVDAAHQLIANGVKAVAEGANMPTTIEATELFQQAGVLFAPGKAANAGGVATSGLEMAQNAARLGWKAEKVDARLHHIMLDIHHACVEHGGEGEQTNYVQGANIAGFVKVADAMLAQGVI

SEQ ID NO: 64 - DNA, 639 bp, Escherichia coli
  source 1..639: mol_type = genomic DNA
gtgttcgctgaatacggggttctgaattactggacctatctggttggggccatttttattgtgttggtgccagggccaaataccctgtttgtactcaaaaatagcgtcagtagcggtatgaaaggcggttatcttgcggcctgcggtgtatttattggcgatgcggtattgatgtttctggcatgggctggagtggcgacattaattaagaccaccccgatattattcaacattgtacgttatcttggtgcgttttatttgctctatctggggagtaaaattctttacgcgaccctgaagggtaaaaatagcgaggccaaatccgatgagccccaatacggtgctatttttaaacgcgcgttaattttgagcctgactaatccgaaagccattttgttctatgtgtcgtttttcgtacagtttatcgatgttaatgccccacatacgggaatttcattctttattctggcggcgacgctggaactggtgagtttctgctatttgagcttcctgattatatctggtgcttttgtcacgcagtacatacgtaccaaaaagaaactggctaaagttggcaactcactgattggtttgatgttcgtgggtttcgctgcccgactggcgacgctgcaatcctga

SEQ ID NO: 65 - protein, 212 aa, Escherichia coli
MFAEYGVLNYWTYLVGAIFIVLVPGPNTLFVLKNSVSSGMKGGYLAACGVFIGDAVLMFLAWAGVATLIKTTPILFNIVRYLGAFYLLYLGSKILYATLKGKNSEAKSDEPQYGAIFKRALILSLTNPKAILFYVSFFVQFIDVNAPHTGISFFILAATLELVSFCYLSFLIISGAFVTQYIRTKKKLAKVGNSLIGLMFVGFAARLATLQS

SEQ ID NO: 66 - DNA, 492 bp, Escherichia coli
  source 1..492: mol_type = genomic DNA
atgcgccggatattatcagtcttactcgaaaatgaatcaggcgcgttatcccgcgtgattggccttttttcccagcgtggctacaacattgaaagcctgaccgttgcgccaaccgacgatccgacattatcgcgtatgaccatccagaccgtgggcgatgaaaaagtacttgagcagatcgaaaagcaattacacaaactggtcgatgtcttgcgcgtgagtgagttggggcagggcgcgcatgttgagcgggaaatcatgctggtgaaaattcaggccagcggttacgggcgtgacgaagtgaaacgtaatacggaaatattccgtgggcaaattatcgatgtcacaccctcgctttataccgttcaattagcaggcaccagcggtaagcttgatgcatttttagcatcgattcgcgatgtggcgaaaattgtggaggttgctcgctctggtgtggtcggactttcgcgcggcgataaaataatgcgttga

SEQ ID NO: 67 - protein, 163 aa, Escherichia coli
MRRILSVLLENESGALSRVIGLFSQRGYNIESLTVAPTDDPTLSRMTIQTVGDEKVLEQIEKQLHKLVDVLRVSELGQGAHVEREIMLVKIQASGYGRDEVKRNTEIFRGQIIDVTPSLYTVQLAGTSGKLDAFLASIRDVAKIVEVARSGVVGLSRGDKIMR

SEQ ID NO: 68 - DNA, 492 bp, synthetic construct
  misc_feature 1..492: note = ilvH* gene (G14D/S17F)
  source 1..492: mol_type = other DNA
atgcgccggatattatcagtcttactcgaaaatgaatcagacgcgttattccgcgtgattggccttttttcccagcgtggctacaacattgaaagcctgaccgttgcgccaaccgacgatccgacattatcgcgtatgaccatccagaccgtgggcgatgaaaaagtacttgagcagatcgaaaagcaattacacaaactggtcgatgtcttgcgcgtgagtgagttggggcagggcgcgcatgttgagcgggaaatcatgctggtgaaaattcaggccagcggttacgggcgtgacgaagtgaaacgtaatacggaaatattccgtgggcaaattatcgatgtcacaccctcgctttataccgttcaattagcaggcaccagcggtaagcttgatgcatttttagcatcgattcgcgatgtggcgaaaattgtggaggttgctcgctctggtgtggtcggactttcgcgcggcgataaaataatgcgttga

SEQ ID NO: 69 - protein, 163 aa, synthetic construct
  REGION 1..163: note = IlvH* protein (G14D/S17F)
MRRILSVLLENESDALFRVIGLFSQRGYNIESLTVAPTDDPTLSRMTIQTVGDEKVLEQIEKQLHKLVDVLRVSELGQGAHVEREIMLVKIQASGYGRDEVKRNTEIFRGQIIDVTPSLYTVQLAGTSGKLDAFLASIRDVAKIVEVARSGVVGLSRGDKIMR

SEQ ID NO: 70 - DNA, 276 bp, synthetic construct
  misc_feature 1..276: note = ilvH* gene (N29K/Q92STOP)
  source 1..276: mol_type = other DNA
atgcgccggatattatcagtcttactcgaaaatgaatcaggcgcgttatcccgcgtgattggccttttttcccagcgtggctacaaaattgaaagcctgaccgtggcgccaaccgacgatccgacattatcgcgtatgaccatccagaccgtgggcgatgaaaaagtacttgagcagatcgaaaagcaattacacaaactggtcgatgtcttgcgcgtgagtgagttggggcagggcgcgcatgttgagcgggaaatcatgctggtgaaaatttag

SEQ ID NO: 71 - protein, 91 aa, synthetic construct
  REGION 1..91: note = IlvH* protein (N29K/Q92STOP)
MRRILSVLLENESGALSRVIGLFSQRGYKIESLTVAPTDDPTLSRMTIQTVGDEKVLEQIEKQLHKLVDVLRVSELGQGAHVEREIMLVKI

SEQ ID NO: 72 - DNA, 1725 bp, Escherichia coli
  source 1..1725: mol_type = genomic DNA
atggagatgttgtctggagccgagatggtcgtccgatcgcttatcgatcagggcgttaaacaagtattcggttatcccggaggcgcagtccttgatatttatgatgcattgcataccgtgggtggtattgatcatgtattagttcgtcatgagcaggcggcggtgcatatggccgatggcctggcgcgcgcgaccggggaagtcggcgtcgtgctggtaacgtcgggtccaggggcgaccaatgcgattactggcatcgccaccgcttatatggattccattccattagttgtcctttccgggcaggtagcgacctcgttgataggttacgatgcctttcaggagtgcgacatggtggggatttcgcgaccggtggttaaacacagttttctggttaagcaaacggaagacattccgcaggtgctgaaaaaggctttctggctggcggcaagtggtcgcccaggaccagtagtcgttgatttaccgaaagatattcttaatccggcgaacaaattaccctatgtctggccggagtcggtcagtatgcgttcttacaatcccactactaccggacataaagggcaaattaagcgtgctctgcaaacgctggtagcggcaaaaaaaccggttgtctacgtaggcggtggggcaatcacggcgggctgccatcagcagttgaaagaaacggtggaggcgttgaatctgcccgttgtttgctcattgatggggctgggggcgtttccggcaacgcatcgtcaggcactgggcatgctgggaatgcacggtacctacgaagccaatatgacgatgcataacgcggatgtgattttcgccgtcggggtacgatttgatgaccgaacgacgaacaatctggcaaagtactgcccaaatgccactgttctgcatatcgatattgatcctacttccatttctaaaaccgtgactgcggatatcccgattgtgggggatgctcgccaggtcctcgaacaaatgcttgaactcttgtcgcaagaatccgcccatcaaccactggatgagatccgcgactggtggcagcaaattgaacagtggcgcgctcgtcagtgcctgaaatatgacactcacagtgaaaagattaaaccgcaggcggtgatcgagactctttggcggttgacgaagggagacgcttacgtgacgtccgatgtcgggcagcaccagatgtttgctgcactttattatccattcgacaaaccgcgtcgctggatcaattccggtggcctcggcacgatgggttttggtttacctgcggcactgggcgtcaaaatggcgttgccagaagaaaccgtggtttgcgtcactggcgacggcagtattcagatgaacatccaggaactgtctaccgcgttgcaatacgagttgcccgtactggtggtgaatctcaataaccgctatctggggatggtgaagcagtggcaggacatgatctattccggccgtcattcacaatcttatatgcaatcgctacccgatttcgtccgtctggcggaagcctatgggcatgtcgggatccagatttctcatccgcatgagctggaaagcaaacttagcgaggcgctggaacaggtgcgcaataatcgcctggtgtttgttgatgttaccgtcgatggcagcgagcacgtctacccgatgcagattcgcgggggcggaatggatgaaatgtggttaagcaaaacggagagaacctga

SEQ ID NO: 73 - protein, 574 aa, Escherichia coli
MEMLSGAEMVVRSLIDQGVKQVFGYPGGAVLDIYDALHTVGGIDHVLVRHEQAAVHMADGLARATGEVGVVLVTSGPGATNAITGIATAYMDSIPLVVLSGQVATSLIGYDAFQECDMVGISRPVVKHSFLVKQTEDIPQVLKKAFWLAASGRPGPVVVDLPKDILNPANKLPYVWPESVSMRSYNPTTTGHKGQIKRALQTLVAAKKPVVYVGGGAITAGCHQQLKETVEALNLPVVCSLMGLGAFPATHRQALGMLGMHGTYEANMTMHNADVIFAVGVRFDDRTTNNLAKYCPNATVLHIDIDPTSISKTVTADIPIVGDARQVLEQMLELLSQESAHQPLDEIRDWWQQIEQWRARQCLKYDTHSEKIKPQAVIETLWRLTKGDAYVTSDVGQHQMFAALYYPFDKPRRWINSGGLGTMGFGLPAALGVKMALPEETVVCVTGDGSIQMNIQELSTALQYELPVLVVNLNNRYLGMVKQWQDMIYSGRHSQSYMQSLPDFVRLAEAYGHVGIQISHPHELESKLSEALEQVRNNRLVFVDVTVDGSEHVYPMQIRGGGMDEMWLSKTERT

SEQ ID NO: 74 - DNA, 1476 bp, Methanocaldococcus jannaschii
  source 1..1476: mol_type = genomic DNA
atgatggtaaggatatttgatacaacacttagagatggagagcaaacaccaggagtttctttaacaccaaatgataagttagagatagcaaaaaaattggatgagcttggagttgatgttatagaggcaggttcagctataacttcaaaaggagagagagaaggaataaaattaataacaaaagaaggtttaaatgcagaaatctgctcatttgttagagctttacctgtagatattgatgctgccttagaatgtgatgtagatagtgtccatttagtagtgccaacatctccaatacacatgaaatataagcttagaaaaacagaagatgaggttttagagacagctttaaaggctgtagagtatgctaaagaacatggattgattgttgagttatctgcagaggatgcaacaagaagtgatgtaaatttcttaataaaactatttaatgaaggggaaaaggttggagcagacagagtttgtgtttgtgacacagtaggagttttaactccacaaaagagtcaggaattatttaaaaaaataactgaaaatgttaatttaccggtctcagttcattgccacaacgactttggaatggctactgctaatacttgctcagcagttttaggtggagctgttcagtgccacgtaacagttaatggtattggagagagagcaggaaatgcctcattggaagaggttgttgctgctttaaaaatactctatggctatgatactaagataaagatggaaaagttatatgaggtttcaagaattgtctcaagattgatgaaacttcctgttccaccaaataaagcaattgttggggacaatgcatttgctcatgaagcaggaatacatgttgatggattaataaaaaatactgaaacctatgagccaataaaaccagaaatggttgggaatagaagaagaattattttgggtaagcattctggtagaaaagctttaaaatacaaacttgatttgatgggcataaacgttagtgatgagcaattaaataaaatatatgaaagagttaaagaatttggggatttgggtaaatacatttcagacgctgatttgttggctatagttagagaagttactggaaaattggtagaagagaaaatcaaattagatgaattaactgttgtatctggaaataaaataacaccaattgcatctgttaaactccattataaaggagaagatataactttaatagaaactgcttatggtgttggaccggtagatgcagcaataaatgctgtgagaaaggcaataagtggagttgcagatattaagttggtagagtatagagttgaagcaattggtggaggaactgatgcgttaatagaggttgttgttaaattaagaaaaggaactgaaattgttgaagttagaaaatcagacgctgatataataagggcttctgtagatgctgtaatggaaggaatcaatatgttattgaattaa

SEQ ID NO: 75 - protein, 491 aa, Methanocaldococcus jannaschii
MMVRIFDTTLRDGEQTPGVSLTPNDKLEIAKKLDELGVDVIEAGSAITSKGEREGIKLITKEGLNAEICSFVRALPVDIDAALECDVDSVHLVVPTSPIHMKYKLRKTEDEVLETALKAVEYAKEHGLIVELSAEDATRSDVNFLIKLFNEGEKVGADRVCVCDTVGVLTPQKSQELFKKITENVNLPVSVHCHNDFGMATANTCSAVLGGAVQCHVTVNGIGERAGNASLEEVVAALKILYGYDTKIKMEKLYEVSRIVSRLMKLPVPPNKAIVGDNAFAHEAGIHVDGLIKNTETYEPIKPEMVGNRRRIILGKHSGRKALKYKLDLMGINVSDEQLNKIYERVKEFGDLGKYISDADLLAIVREVTGKLVEEKIKLDELTVVSGNKITPIASVKLHYKGEDITLIETAYGVGPVDAAINAVRKAISGVADIKLVEYRVEAIGGGTDALIEVVVKLRKGTEIVEVRKSDADIIRASVDAVMEGINMLLN

SEQ ID NO: 76 - DNA, 1119 bp, synthetic construct
  misc_feature 1..1119: note = cimA* gene
  source 1..1119: mol_type = other DNA
atgatggtaaggatatttgatacaacacttagagatggagagcaaacaccaggagtttctttaacaccaaatgataagttagagatagcaaaaaaattggatgagcttggagttgatgttatagaggcaggttcagctgtaacttcaaaaggagagagagaaggaataaaattaataacaaaagaaggtttaaatgcagaaatctgctcatttgttagagctttacctgtagatattgatgctgccttagaatgtgatgtagatagtgtccatttagtagtgccaacatctccaatacacatgaaatataagcttagaaaaacagaagatgaggttttagtgacagctttaaaggctgtagagtatgctaaagaacagggattgattgttgagttatctgcagaggatgcaacaagaagtgatgtaaatttcttaataaaactatttaatgaaggggaaaaggttggagcagacagagtttgtgtttgtgacacagtaggagttttaactccacaaaagagtcaggaattatttaaaaaaataactgaaaatgttaatttaccggtctcagttcattgccacaacgactttggaatggctactgctaatgcttgctcagcagttttaggtggagctgttcagtgccacgtaacagttaatggtattggagagagagcaggaaatgcctcattggaagaggttgttgctgctagcaaaatactctatggctatgatactaagataaagatggaaaagttatatgaggtttcaagaattgtctcaagattgatgaaacttcctgttccaccaaataaagcaattgttggggacaatgcatttgctcatgaagcaggaatacatgttgatggattaataaaaaatactgaaacctatgagccaataaaaccagaaatggttgggaatagaagaagaattattttgggtaagcattctggtagaaaagctttaaaatacaaacttgatttgatgggcataaacgttagtgatgagcaattaaataaaatatatgaaagagttaaagaatttggggatttgggtaaatacatttcagacgctgatttgttggctatagttagagaagttactggaaaattgtga

SEQ ID NO: 77 - protein, 372 aa, synthetic construct
  REGION 1..372: note = cimA* protein
MMVRIFDTTLRDGEQTPGVSLTPNDKLEIAKKLDELGVDVIEAGSAVTSKGEREGIKLITKEGLNAEICSFVRALPVDIDAALECDVDSVHLVVPTSPIHMKYKLRKTEDEVLVTALKAVEYAKEQGLIVELSAEDATRSDVNFLIKLFNEGEKVGADRVCVCDTVGVLTPQKSQELFKKITENVNLPVSVHCHNDFGMATANACSAVLGGAVQCHVTVNGIGERAGNASLEEVVAASKILYGYDTKIKMEKLYEVSRIVSRLMKLPVPPNKAIVGDNAFAHEAGIHVDGLIKNTETYEPIKPEMVGNRRRIILGKHSGRKALKYKLDLMGINVSDEQLNKIYERVKEFGDLGKYISDADLLAIVREVTGKL

SEQ ID NO: 78 - DNA, 738 bp, Escherichia coli
  source 1..738: mol_type = genomic DNA
atggaaagccctactccacagcctgctcctggttcggcgaccttcatggaaggatgcaaagacagtttaccgattgttattagttatattccggtggcctttgcgttcggtctgaatgcgacccgtctgggattctctcctctcgaaagcgtttttttctcctgcatcatttatgcaggcgcgagccagttcgtcattaccgcgatgctggcagccgggagtagtttgtggattgctgcactgaccgtcatggcaatggatgttcgccatgtgttgtatggcccgtcactgcgtagccgtattattcagcgtctgcaaaaatcgaaaaccgccctgtgggcgtttggcctgacggatgaggtttttgccgccgcaaccgcaaaactggtacgcaataatcgccgctggagcgagaactggatgatcggcattgccttcagttcatggtcatcgtgggtatttggtacggtaataggggcattctccggcagcggcttgctgcaaggttatcccgccgttgaagctgcattaggttttatgcttccggcactctttatgagtttcctgctcgcctctttccagcgcaaacaatctctttgcgttaccgcagcgttagttggtgcccttgcaggcgtaacgctattttctattcccgtcgccattctggcaggcattgtctgtggctgcctcactgcgttaatccaggcattctggcaaggagcgcccgatgagctatga

SEQ ID NO: 79 - protein, 245 aa, Escherichia coli
MESPTPQPAPGSATFMEGCKDSLPIVISYIPVAFAFGLNATRLGFSPLESVFFSCIIYAGASQFVITAMLAAGSSLWIAALTVMAMDVRHVLYGPSLRSRIIQRLQKSKTALWAFGLTDEVFAAATAKLVRNNRRWSENWMIGIAFSSWSSWVFGTVIGAFSGSGLLQGYPAVEAALGFMLPALFMSFLLASFQRKQSLCVTAALVGALAGVTLFSIPVAILAGIVCGCLTALIQAFWQGAPDEL

SEQ ID NO: 80 - DNA, 336 bp, Escherichia coli
  source 1..336: mol_type = genomic DNA
atgagctatgaggttctgctgcttgggttactagttggcgtggcgaattattgcttccgctatttgccgctgcgcctgcgtgtgggtaatgcccgcccaaccaaacgtggcgcggtaggtattttgctcgacaccattggcatcgcctcgatatgcgctctgctggttgtctctaccgcaccagaagtgatgcacgatacacgccgtttcgtgcccacgctggtcggcttcgcggtactgggtgccagtttctataaaacacgcagcattatcatcccaacactgcttagtgcgctggcctatgggctcgcctggaaagtgatggcgattatataa

SEQ ID NO: 81 - protein, 111 aa, Escherichia coli
MSYEVLLLGLLVGVANYCFRYLPLRLRVGNARPTKRGAVGILLDTIGIASICALLVVSTAPEVMHDTRRFVPTLVGFAVLGASFYKTRSIIIPTLLSALAYGLAWKVMAII

SEQ ID NO: 82 - DNA, 1203 bp, Escherichia coli
  source 1..1203: mol_type = genomic DNA
atgtcgagtaagttagtactggttctgaactgcggtagttcttcactgaaatttgccatcatcgatgcagtaaatggtgaagagtacctttctggtttagccgaatgtttccacctgcccgaagcacgtatcaaatggaaaatggacggcaataaacaggaagcggctttaggtgcaggcgccgctcacagcgaagcgctcaactttatcgttaatactattctggcacaaaaaccagaactgtctgcgcagctgactgctatcggtcaccgtatcgtacacggcggcgaaaagtataccagctccgtagtgatcgatgagtctgttattcagggtatcaaagatgcagcttcttttgcaccgctgcacaacccggctcacctgatcggtatcgaagaagctctgaaatctttcccacagctgaaagacaaaaacgttgctgtatttgacaccgcgttccaccagactatgccggaagagtcttacctctacgccctgccttacaacctgtacaaagagcacggcatccgtcgttacggcgcgcacggcaccagccacttctatgtaacccaggaagcggcaaaaatgctgaacaaaccggtagaagaactgaacatcatcacctgccacctgggcaacggtggttccgtttctgctatccgcaacggtaaatgcgttgacacctctatgggcctgaccccgctggaaggtctggtcatgggtacccgttctggtgatatcgatccggcgatcatcttccacctgcacgacaccctgggcatgagcgttgacgcaatcaacaaactgctgaccaaagagtctggcctgctgggtctgaccgaagtgaccagcgactgccgctatgttgaagacaactacgcgacgaaagaagacgcgaagcgcgcaatggacgtttactgccaccgcctggcgaaatacatcggtgcctacactgcgctgatggatggtcgtctggacgctgttgtattcactggtggtatcggtgaaaatgccgcaatggttcgtgaactgtctctgggcaaactgggcgtgctgggctttgaagttgatcatgaacgcaacctggctgcacgtttcggcaaatctggtttcatcaacaaagaaggtacccgtcctgcggtggttatcccaaccaacgaagaactggttatcgcgcaagacgcgagccgcctgactgcctga

SEQ ID NO: 83 - protein, 400 aa, Escherichia coli
MSSKLVLVLNCGSSSLKFAIIDAVNGEEYLSGLAECFHLPEARIKWKMDGNKQEAALGAGAAHSEALNFIVNTILAQKPELSAQLTAIGHRIVHGGEKYTSSVVIDESVIQGIKDAASFAPLHNPAHLIGIEEALKSFPQLKDKNVAVFDTAFHQTMPEESYLYALPYNLYKEHGIRRYGAHGTSHFYVTQEAAKMLNKPVEELNIITCHLGNGGSVSAIRNGKCVDTSMGLTPLEGLVMGTRSGDIDPAIIFHLHDTLGMSVDAINKLLTKESGLLGLTEVTSDCRYVEDNYATKEDAKRAMDVYCHRLAKYIGAYTALMDGRLDAVVFTGGIGENAAMVRELSLGKLGVLGFEVDHERNLAARFGKSGFINKEGTRPAVVIPTNEELVIAQDASRLTA

SEQ ID NO: 84 - DNA, 1959 bp, Escherichia coli
  source 1..1959: mol_type = genomic DNA
atgagccaaattcacaaacacaccattcctgccaacatcgcagaccgttgcctgataaaccctcagcagtacgaggcgatgtatcaacaatctattaacgtacctgataccttctggggcgaacagggaaaaattcttgactggatcaaaccttaccagaaggtgaaaaacacctcctttgcccccggtaatgtgtccattaaatggtacgaggacggcacgctgaatctggcggcaaactgccttgaccgccatctgcaagaaaacggcgatcgtaccgccatcatctgggaaggcgacgacgccagccagagcaaacatatcagctataaagagctgcaccgcgacgtctgccgcttcgccaataccctgctcgagctgggcattaaaaaaggtgatgtggtggcgatttatatgccgatggtgccggaagccgcggttgcgatgctggcctgcgcccgcattggcgcggtgcattcggtgattttcggcggcttctcgccggaagccgttgccgggcgcattattgattccaactcacgactggtgatcacttccgacgaaggtgtgcgtgccgggcgcagtattccgctgaagaaaaacgttgatgacgcgctgaaaaacccgaacgtcaccagcgtagagcatgtggtggtactgaagcgtactggcgggaaaattgactggcaggaagggcgcgacctgtggtggcacgacctggttgagcaagcgagcgatcagcaccaggcggaagagatgaacgccgaagatccgctgtttattctctacacctccggttctaccggtaagccaaaaggtgtgctgcatactaccggcggttatctggtgtacgcggcgctgacctttaaatatgtctttgattatcatccgggtgatatctactggtgcaccgccgatgtgggctgggtgaccggacacagttacttgctgtacggcccgctggcctgcggtgcgaccacgctgatgtttgaaggcgtacccaactggccgacgcctgcccgtatggcgcaggtggtggacaagcatcaggtcaatattctctataccgcacccacggcgatccgcgcgctgatggcggaaggcgataaagcgatcgaaggcaccgaccgttcgtcgctgcgcattctcggttccgtgggcgagccaattaacccggaagcgtgggagtggtactggaaaaaaatcggcaacgagaaatgtccggtggtcgatacctggtggcagaccgaaaccggcggtttcatgatcaccccgctgcctggcgctaccgagctgaaagccggttcggcaacacgtccgttcttcggcgtgcaaccggcgctggtcgataacgaaggtaacccgctggagggggccaccgaaggtagcctggtaatcaccgactcctggccgggtcaggcgcgtacgctgtttggcgatcacgaacgttttgaacagacctacttctccaccttcaaaaatatgtatttcagcggcgacggcgcgcgtcgcgatgaagatggctattactggataaccgggcgtgtggacgacgtgctgaacgtctccggtcaccgtctggggacggcagagattgagtcggcgctggtggcgcatccgaagattgccgaagccgccgtagtaggtattccgcacaatattaaaggtcaggcgatctacgcctacgtcacgcttaatcacggggaggaaccgtcaccagaactgtacgcagaagtccgcaactgggtgcgtaaagagattggcccgctggcgacgccagacgtgctgcactggaccgactccctgcctaaaacccgctccggcaaaattatgcgccgtattctgcgcaaaattgcggcgggcgataccagcaacctgggcgatacctcgacgcttgccgatcctggcgtagtcgagaagctgcttgaagagaagcaggctatcgcgatgccatcgtaa

SEQ ID NO: 85 - protein, 652 aa, Escherichia coli
MSQIHKHTIPANIADRCLINPQQYEAMYQQSINVPDTFWGEQGKILDWIKPYQKVKNTSFAPGNVSIKWYEDGTLNLAANCLDRHLQENGDRTAIIWEGDDASQSKHISYKELHRDVCRFANTLLELGIKKGDVVAIYMPMVPEAAVAMLACARIGAVHSVIFGGFSPEAVAGRIIDSNSRLVITSDEGVRAGRSIPLKKNVDDALKNPNVTSVEHVVVLKRTGGKIDWQEGRDLWWHDLVEQASDQHQAEEMNAEDPLFILYTSGSTGKPKGVLHTTGGYLVYAALTFKYVFDYHPGDIYWCTADVGWVTGHSYLLYGPLACGATTLMFEGVPNWPTPARMAQVVDKHQVNILYTAPTAIRALMAEGDKAIEGTDRSSLRILGSVGEPINPEAWEWYWKKIGNEKCPVVDTWWQTETGGFMITPLPGATELKAGSATRPFFGVQPALVDNEGNPLEGATEGSLVITDSWPGQARTLFGDHERFEQTYFSTFKNMYFSGDGARRDEDGYYWITGRVDDVLNVSGHRLGTAEIESALVAHPKIAEAAVVGIPHNIKGQAIYAYVTLNHGEEPSPELYAEVRNWVRKEIGPLATPDVLHWTDSLPKTRSGKIMRRILRKIAAGDTSNLGDTSTLADPGVVEKLLEEKQAIAMPS

SEQ ID NO: 86 - DNA, 1320 bp, Escherichia coli
  source 1..1320: mol_type = genomic DNA
atgacccatcaattaagatcgcgcgatatcatcgctctgggctttatgacatttgcgttgttcgtcggcgcaggtaacattattttccctccaatggtcggcttacaggcaggcgaacacgtctggactgcggcattcggcttcctcattactgccgttggcctgccggtattaacggtagtggcgctggcaaaagttggcggcggtgttgacagcctcagcacgccaatcggtaaagtcgctggcgtactgctggcaacggtttgttacctggcggtggggccgcttttcgctacgccgcgtacagctaccgtttcctttgaagtggggattgcgccgctgacgggtgattccgcgctgccgctgtttatctacagcctggtctatttcgctatcgttattctggtttcgctctatccgggcaagctgctggataccgtgggcaacttccttgcgccgctgaaaattatcgcgctggtcatcctgtctgttgccgctattgtctggccggcgggttctatcagcacggcgactgaggcttatcaaaacgctgcgttttctaacggcttcgttaacggctatctgaccatggatacgctgggcgcaatggtgtttggtatcgttattgttaacgcggcgcgttctcgtggcgttaccgaagcgcgtctgctgacccgttataccgtctgggctggcctgatggcgggtgttggtctgactctgctgtacctggcgctgttccgtctggggtcagacagcgcgtcgctggtcgatcagtctgcaaacggcgctgctattctgcatgcttacgttcagcacacctttggcggcggcggtagcttcctgctggcggcgttaatcttcatcgcctgcctggtaacggcagttggcctgacctgtgcttgtgcagaattctttgcccagtacgtaccgctctcttatcgtacgctggtgtttatcctcggcggcttctcgatggtggtttctaacctcggcttaagccagctgatccagatctccgtaccggtgctgaccgctatttatccgccgtgtatcgcactggttgtattaagttttacacgctcatggtggcataattcgtcccgcgtgattgctccgccgatgtttatcagcctgctttttggtattctcgacgggatcaaagcatctgcattcagcgatatcttaccgtcctgggcgcagcgtttaccgctggccgaacaaggtctggcgtggttaatgccaacagtggtgatggtggttctggccattatctgggatcgcgcggcaggtcgtcaggtgacctccagcgctcactaa

SEQ ID NO: 87 - protein, 439 aa, Escherichia coli
MTHQLRSRDIIALGFMTFALFVGAGNIIFPPMVGLQAGEHVWTAAFGFLITAVGLPVLTVVALAKVGGGVDSLSTPIGKVAGVLLATVCYLAVGPLFATPRTATVSFEVGIAPLTGDSALPLFIYSLVYFAIVILVSLYPGKLLDTVGNFLAPLKIIALVILSVAAIVWPAGSISTATEAYQNAAFSNGFVNGYLTMDTLGAMVFGIVIVNAARSRGVTEARLLTRYTVWAGLMAGVGLTLLYLALFRLGSDSASLVDQSANGAAILHAYVQHTFGGGGSFLLAALIFIACLVTAVGLTCACAEFFAQYVPLSYRTLVFILGGFSMVVSNLGLSQLIQISVPVLTAIYPPCIALVVLSFTRSWWHNSSRVIAPPMFISLLFGILDGIKASAFSDILPSWAQRLPLAEQGLAWLMPTVVMVVLAIIWDRAAGRQVTSSAH

SEQ ID NO: 88 - DNA, 1110 bp, Escherichia coli
  source 1..1110: mol_type = genomic DNA
atgaaacggaatgcgaaaactatcatcgcagggatgattgcactggcaatttcacacaccgctatggctgacgatattaaagtcgccgttgtcggcgcgatgtccggcccgattgcccagtggggcgatatggaatttaacggcgcgcgtcaggcaattaaagacattaatgccaaagggggaattaagggcgataaactggttggcgtggaatatgacgacgcatgcgacccgaaacaagccgttgcggtcgccaacaaaatcgttaatgacggcattaaatacgttattggtcatctgtgttcttcttctacccagcctgcgtcagatatctatgaagacgaaggtattctgatgatctcgccgggagcgaccaacccggagctgacccaacgcggttatcaacacattatgcgtactgccgggctggactcttcccaggggccaacggcggcaaaatacattcttgagacggtgaagccccagcgcatcgccatcattcacgacaaacaacagtatggcgaagggctggcgcgttcggtgcaggacgggctgaaagcggctaacgccaacgtcgtcttcttcgacggtattaccgccggggagaaagatttctccgcgctgatcgcccgcctgaaaaaagaaaacatcgacttcgtttactacggcggttactacccggaaatggggcagatgctgcgccaggcccgttccgttggcctgaaaacccagtttatggggccggaaggtgtgggtaatgcgtcgttgtcgaacattgccggtgatgccgccgaaggcatgttggtcactatgccaaaacgctatgaccaggatccggcaaaccagggcatcgttgatgcgctgaaagcagacaagaaagatccgtccgggccttatgtctggatcacctacgcggcggtgcaatctctggcgactgcccttgagcgtaccggcagcgatgagccgctggcgctggtgaaagatttaaaagctaacggtgcaaacaccgtgattgggccgctgaactgggatgaaaaaggcgatcttaagggatttgattttggtgtcttccagtggcacgccgacggttcatccacggcagccaagtga

SEQ ID NO: 89 - protein, 369 aa, Escherichia coli
MKRNAKTIIAGMIALAISHTAMADDIKVAVVGAMSGPIAQWGDMEFNGARQAIKDINAKGGIKGDKLVGVEYDDACDPKQAVAVANKIVNDGIKYVIGHLCSSSTQPASDIYEDEGILMISPGATNPELTQRGYQHIMRTAGLDSSQGPTAAKYILETVKPQRIAIIHDKQQYGEGLARSVQDGLKAANANVVFFDGITAGEKDFSALIARLKKENIDFVYYGGYYPEMGQMLRQARSVGLKTQFMGPEGVGNASLSNIAGDAAEGMLVTMPKRYDQDPANQGIVDALKADKKDPSGPYVWITYAAVQSLATALERTGSDEPLALVKDLKANGANTVIGPLNWDEKGDLKGFDFGVFQWHADGSSTAAK

SEQ ID NO: 90 - DNA, 927 bp, Escherichia coli
  source 1..927: mol_type = genomic DNA
atgtctgagcagtttttgtatttcttgcagcagatgtttaacggcgtcacgctgggcagtacctacgcgctgatagccatcggctacaccatggtttacggcattatcggcatgatcaacttcgcccacggcgaggtttatatgattggcagctacgtctcatttatgatcatcgccgcgctgatgatgatgggcattgataccggctggctgctggtagctgcgggattcgtcggcgcaatcgtcattgccagcgcctacggctggagtatcgaacgggtggcttaccgcccggtgcgtaactctaagcgcctgattgcactcatctctgcaatcggtatgtccatcttcctgcaaaactacgtcagcctgaccgaaggttcgcgcgacgtggcgctgccgagcctgtttaacggtcagtgggtggtggggcatagcgaaaacttctctgcctctattaccaccatgcaggcggtgatctggattgttaccttcctcgccatgctggcgctgacgattttcattcgctattcccgcatgggtcgcgcgtgtcgtgcctgcgcggaagatctgaaaatggcgagtctgcttggcattaacaccgaccgggtgattgcgctgacctttgtgattggcgcggcgatggcggcggtggcgggtgtgctgctcggtcagttctacggcgtcattaacccctacatcggctttatggccgggatgaaagcctttaccgcggcggtgctcggtgggattggcagcattccgggagcgatgattggcggcctgattctggggattgcggaggcgctctcttctgcctatctgagtacggaatataaagatgtggtctcattcgccctgctgattctggtgctgctggtgatgccgaccggtattctgggtcgcccggaggtagagaaagtatga

SEQ ID NO: 91 - protein, 308 aa, Escherichia coli
MSEQFLYFLQQMFNGVTLGSTYALIAIGYTMVYGIIGMINFAHGEVYMIGSYVSFMIIAALMMMGIDTGWLLVAAGFVGAIVIASAYGWSIERVAYRPVRNSKRLIALISAIGMSIFLQNYVSLTEGSRDVALPSLFNGQWVVGHSENFSASITTMQAVIWIVTFLAMLALTIFIRYSRMGRACRACAEDLKMASLLGINTDRVIALTFVIGAAMAAVAGVLLGQFYGVINPYIGFMAGMKAFTAAVLGGIGSIPGAMIGGLILGIAEALSSAYLSTEYKDVVSFALLILVLLVMPTGILGRPEVEKV

SEQ ID NO: 92 - DNA, 1278 bp, Escherichia coli
  source 1..1278: mol_type = genomic DNA
atgaaaccgatgcatattgcaatggcgctgctctctgccgcgatgttctttgtgctggcgggcgtctttatgggcgtgcaactggagctggatggcaccaaactggtggtcgacacggcttcggatgtccgttggcagtgggtgtttatcggcacggcggtggtctttttcttccagcttttgcgaccggctttccagaaagggttgaaaagcgtttccggaccgaagtttattctgcccgccattgatggctccacggtgaagcagaaactgttcctcgtggcgctgttggtgcttgcggtggcgtggccgtttatggtttcacgcgggacggtggatattgccaccctgaccatgatctacattatcctcggtctcgggctgaacgtggttgttggtctttctggtctgctggtgctggggtacggcggtttttacgccatcggcgcttacacttttgcgctgctcaatcactattacggcttgggcttctggacctgcctgccgattgctggattaatggcagcggcggcgggcttcctgctcggttttccggtgctgcgtttgcgcggtgactatctggcgatcgttaccctcggtttcggcgaaattgtgcgcatattgctgctcaataacaccgaaattaccggcggcccgaacggaatcagtcagatcccgaaaccgacactcttcggactcgagttcagccgtaccgctcgtgaaggcggctgggacacgttcagtaatttctttggcctgaaatacgatccctccgatcgtgtcatcttcctctacctggtggcgttgctgctggtggtgctaagcctgtttgtcattaaccgcctgctgcggatgccgctggggcgtgcgtgggaagcgttgcgtgaagatgaaatcgcctgccgttcgctgggcttaagcccgcgtcgtatcaagctgactgcctttaccataagtgccgcgtttgccggttttgccggaacgctgtttgcggcgcgtcagggctttgtcagcccggaatccttcacctttgccgaatcggcgtttgtgctggcgatagtggtgctcggcggtatgggctcgcaatttgcggtgattctggcggcaattttgctggtggtgtcgcgcgagttgatgcgtgatttcaacgaatacagcatgttaatgctcggtggtttgatggtgctgatgatgatctggcgtccgcagggcttgctgcccatgacgcgcccgcaactgaagctgaaaaacggcgcagcgaaaggagagcaggcatga

SEQ ID NO: 93 - protein, 425 aa, Escherichia coli
MKPMHIAMALLSAAMFFVLAGVFMGVQLELDGTKLVVDTASDVRWQWVFIGTAVVFFFQLLRPAFQKGLKSVSGPKFILPAIDGSTVKQKLFLVALLVLAVAWPFMVSRGTVDIATLTMIYIILGLGLNVVVGLSGLLVLGYGGFYAIGAYTFALLNHYYGLGFWTCLPIAGLMAAAAGFLLGFPVLRLRGDYLAIVTLGFGEIVRILLLNNTEITGGPNGISQIPKPTLFGLEFSRTAREGGWDTFSNFFGLKYDPSDRVIFLYLVALLLVVLSLFVINRLLRMPLGRAWEALREDEIACRSLGLSPRRIKLTAFTISAAFAGFAGTLFAARQGFVSPESFTFAESAFVLAIVVLGGMGSQFAVILAAILLVVSRELMRDFNEYSMLMLGGLMVLMMIWRPQGLLPMTRPQLKLKNGAAKGEQA

SEQ ID NO: 94 - DNA, 768 bp, Escherichia coli
  source 1..768: mol_type = genomic DNA
atgagtcagccattattatctgttaacggcctgatgatgcgcttcggcggcctgctggcggtgaacaacgtcaatcttgaactgtacccgcaggagatcgtctcgttaatcggccctaacggtgccggaaaaaccacggtttttaactgtctgaccggattctacaaacccaccggcggcaccattttactgcgcgatcagcacctggaaggtttaccggggcagcaaattgcccgcatgggcgtggtgcgcaccttccagcatgtgcgtctgttccgtgaaatgacggtaattgaaaacctgctggtggcgcagcatcagcaactgaaaaccgggctgttctctggcctgttgaaaacgccatccttccgtcgcgcccagagcgaagcgctcgaccgcgccgcgacctggcttgagcgcattggtttgctggaacacgccaaccgtcaggcgagtaacctggcctatggtgaccagcgccgtcttgagattgcccgctgcatggtgacgcagccggagattttaatgctcgacgaacctgcggcaggtcttaacccgaaagagacgaaagagctggatgagctgattgccgaactgcgcaatcatcacaacaccactatcttgttgattgaacacgatatgaagctggtgatgggaatttcggaccgaatttacgtggtcaatcaggggacgccgctggcaaacggtacgccggagcagatccgtaataacccggacgtgatccgtgcctatttaggtgaggcataa

SEQ ID NO: 95 - protein, 255 aa, Escherichia coli
MSQPLLSVNGLMMRFGGLLAVNNVNLELYPQEIVSLIGPNGAGKTTVFNCLTGFYKPTGGTILLRDQHLEGLPGQQIARMGVVRTFQHVRLFREMTVIENLLVAQHQQLKTGLFSGLLKTPSFRRAQSEALDRAATWLERIGLLEHANRQASNLAYGDQRRLEIARCMVTQPEILMLDEPAAGLNPKETKELDELIAELRNHHNTTILLIEHDMKLVMGISDRIYVVNQGTPLANGTPEQIRNNPDVIRAYLGEA

SEQ ID NO: 96 - DNA, 714 bp, Escherichia coli
  source 1..714: mol_type = genomic DNA
atggaaaaagtcatgttgtcctttgacaaagtcagcgcccactacggcaaaatccaggcgctgcatgaggtgagcctgcatatcaatcagggcgagattgtcacgctgattggcgcgaacggggcggggaaaaccaccttgctcggcacgttatgcggcgatccgcgtgccaccagcgggcgaattgtgtttgatgataaagacattaccgactggcagacagcgaaaatcatgcgcgaagcggtggcgattgtcccggaagggcgtcgcgtcttctcgcggatgacggtggaagagaacctggcgatgggcggtttttttgctgaacgcgaccagttccaggagcgcataaagtgggtgtatgagctgtttccacgtctgcatgagcgccgtattcagcgggcgggcaccatgtccggcggtgaacagcagatgctggcgattggtcgtgcgctgatgagcaacccgcgtttgctactgcttgatgagccatcgctcggtcttgcgccgattatcatccagcaaattttcgacaccatcgagcagctgcgcgagcaggggatgactatctttctcgtcgagcagaacgccaaccaggcgctaaagctggcggatcgcggctacgtgctggaaaacggccatgtagtgctttccgatactggtgatgcgctgctggcgaatgaagcggtgagaagtgcgtatttaggcgggtaa

SEQ ID NO: 97 - protein, 237 aa, Escherichia coli [sequence omitted]

SEQ ID NO: 98 - DNA, 1809 bp, Escherichia coli
  source 1..1809: mol_type = genomic DNA
gtgcaaacctttcaagccgatcttgccattgtaggcgccggtggcgcgggattacgtgctgcaattgctgccgcgcaggcaaatccgaatgcaaaaatcgcactaatctcaaaagtatacccgatgcgtagccataccgttgctgcagaagggggctccgccgctgtcgcgcaggatcatgacagcttcgaatatcactttcacgatacagtagcgggtggcgactggttgtgtgagcaggatgtcgtggattatttcgtccaccactgcccaaccgaaatgacccaactggaactgtggggatgcccatggagccgtcgcccggatggtagcgtcaacgtacgtcgcttcggcggcatgaaaatcgagcgcacctggttcgccgccgataagaccggcttccatatgctgcacacgctgttccagacctctctgcaattcccgcagatccagcgttttgacgaacatttcgtgctggatattctggttgatgatggtcatgttcgcggcctggtagcaatgaacatgatggaaggcacgctggtgcagatccgtgctaacgcggtcgttatggctactggcggtgcgggtcgcgtttatcgttacaacaccaacggcggcatcgttaccggtgacggtatgggtatggcgctaagccacggcgttccgctgcgtgacatggaattcgttcagtatcacccaaccggtctgccaggttccggtatcctgatgaccgaaggttgccgcggtgaaggcggtattctggtcaacaaaaatggctaccgttatctgcaagattacggcatgggcccggaaactccgctgggcgagccgaaaaacaaatatatggaactgggtccacgcgacaaagtctctcaggccttctggcacgaatggcgtaaaggcaacaccatctccacgccgcgtggcgatgtggtttatctcgacttgcgtcacctcggcgagaaaaaactgcatgaacgtctgccgttcatctgcgaactggcgaaagcgtacgttggcgtcgatccggttaaagaaccgattccggtacgtccgaccgcacactacaccatgggcggtatcgaaaccgatcagaactgtgaaacccgcattaaaggtctgttcgccgtgggtgaatgttcctctgttggtctgcacggtgcaaaccgtctgggttctaactccctggcggaactggtggtcttcggccgtctggccggtgaacaagcgacagagcgtgcagcaactgccggtaatggcaacgaagcggcaattgaagcgcaggcagctggcgttgaacaacgtctgaaagatctggttaaccaggatggcggcgaaaactgggcgaagatccgcgacgaaatgggcctggctatggaagaaggctgcggtatctaccgtacgccggaactgatgcagaaaaccatcgacaagctggcagagctgcaggaacgcttcaagcgcgtgcgcatcaccgacacttccagcgtgttcaacaccgacctgctctacaccattgaactgggccacggtctgaacgttgctgaatgtatggcgcactccgcaatggcacgtaaagagtcccgcggcgcgcaccagcgtctggacgaaggttgcaccgagcgtgacgacgtcaacttcctcaaacacaccctcgccttccgcgatgctgatggcacgactcgcctggagtacagcgacgtgaagattactacgctgccgccagctaaacgcgtttacggtggcgaagcggatgcagccgataaggcggaagcagccaataagaaggagaaggcgaatggctga

SEQ ID NO: 99 - protein, 602 aa, Escherichia coli
VQTFQADLAIVGAGGAGLRAAIAAAQANPNAKIALISKVYPMRSHTVAAEGGSAAVAQDHDSFEYHFHDTVAGGDWLCEQDVVDYFVHHCPTEMTQLELWGCPWSRRPDGSVNVRRFGGMKIERTWFAADKTGFHMLHTLFQTSLQFPQIQRFDEHFVLDILVDDGHVRGLVAMNMMEGTLVQIRANAVVMATGGAGRVYRYNTNGGIVTGDGMGMALSHGVPLRDMEFVQYHPTGLPGSGILMTEGCRGEGGILVNKNGYRYLQDYGMGPETPLGEPKNKYMELGPRDKVSQAFWHEWRKGNTISTPRGDVVYLDLRHLGEKKLHERLPFICELAKAYVGVDPVKEPIPVRPTAHYTMGGIETDQNCETRIKGLFAVGECSSVGLHGANRLGSNSLAELVVFGRLAGEQATERAATAGNGNEAAIEAQAAGVEQRLKDLVNQDGGENWAKIRDEMGLAMEEGCGIYRTPELMQKTIDKLAELQERFKRVRITDTSSVFNTDLLYTIELGHGLNVAECMAHSAMARKESRGAHQRLDEGCTERDDVNFLKHTLAFRDADGTTRLEYSDVKITTLPPAKRVYGGEADAADKAEAANKKEKANG

SEQ ID NO: 100 - DNA, 735 bp, Escherichia coli
  source 1..735: mol_type = genomic DNA
atggctgagatgaaaaacctgaaaattgaggtggtgcgctataacccggaagtcgataccgcaccgcatagcgcattctatgaagtgccttatgacgcaactacctcattactggatgcgctgggctacatcaaagacaacctggcaccggacctgagctaccgctggtcctgccgtatggcgatttgtggttcctgcggcatgatggttaacaacgtgccaaaactggcatgtaaaaccttcctgcgtgattacaccgacggtatgaaggttgaagcgttagctaacttcccgattgaacgcgatctggtggtcgatatgacccacttcatcgaaagtctggaagcgatcaaaccgtacatcatcggcaactcccgcaccgcggatcagggtactaacatccagaccccggcgcagatggcgaagtatcaccagttctccggttgcatcaactgtggtttgtgctacgccgcgtgcccgcagtttggcctgaacccagagttcatcggtccggctgccattacgctggcgcatcgttataacgaagatagccgcgaccacggtaagaaggagcgtatggcgcagttgaacagccagaacggcgtatggagctgtactttcgtgggctactgctccgaagtctgcccgaaacacgtcgatccggctgcggccattcagcagggcaaagtagaaagttcgaaagactttcttatcgcgaccctgaaaccacgctaa

SEQ ID NO: 101 - protein, 244 aa, Escherichia coli
MAEMKNLKIEVVRYNPEVDTAPHSAFYEVPYDATTSLLDALGYIKDNLAPDLSYRWSCRMAICGSCGMMVNNVPKLACKTFLRDYTDGMKVEALANFPIERDLVVDMTHFIESLEAIKPYIIGNSRTADQGTNIQTPAQMAKYHQFSGCINCGLCYAACPQFGLNPEFIGPAAITLAHRYNEDSRDHGKKERMAQLNSQNGVWSCTFVGYCSEVCPKHVDPAAAIQQGKVESSKDFLIATLKPR

SEQ ID NO: 102 - DNA, 396 bp, Escherichia coli
  source 1..396: mol_type = genomic DNA
atgacgactaaacgtaaaccgtatgtacggccaatgacgtccacctggtggaaaaaattgccgttttatcgcttttacatgctgcgcgaaggcacggcggttccggctgtgtggttcagcattgaactgattttcgggctgtttgccctgaaaaatggcccggaagcctgggcgggattcgtcgactttttacaaaacccggttatcgtgatcattaacctgatcactctggcggcagctctgctgcacaccaaaacctggtttgaactggcaccgaaagcggccaatatcattgtaaaagacgaaaaaatgggaccagagccaattatcaaaagtctctgggcggtaactgtggttgccaccatcgtaatcctgtttgttgccctgtactggtaa

SEQ ID NO: 103 - protein, 131 aa, Escherichia coli
MTTKRKPYVRPMTSTWWKKLPFYRFYMLREGTAVPAVWFSIELIFGLFALKNGPEAWAGFVDFLQNPVIVIINLITLAAALLHTKTWFELAPKAANIIVKDEKMGPEPIIKSLWAVTVVATIVILFVALYW

SEQ ID NO: 104 - DNA, 360 bp, Escherichia coli
  source 1..360: mol_type = genomic DNA
atgattaatccaaatccaaagcgttctgacgaaccggtattctggggcctcttcggggccggtggtatgtggagcgccatcattgcgccggtgatgatcctgctggtgggtattctgctgccactggggttgtttccgggtgatgcgctgagctacgagcgcgttctggcgttcgcgcagagcttcattggtcgcgtattcctgttcctgatgatcgttctgccgctgtggtgtggtttacaccgtatgcaccacgcgatgcacgatctgaaaatccacgtacctgcgggcaaatgggttttctacggtctggctgctatcctgacagttgtcacgctgattggtgtcgttacaatctaa

SEQ ID NO: 105 - protein, 119 aa, Escherichia coli
MINPNPKRSDEPVFWGLFGAGGMWSAIIAPVMILLVGILLPLGLFPGDALSYERVLAFAQSFIGRVFLFLMIVLPLWCGLHRMHHAMHDLKIHVPAGKWVFYGLAAILTVVTLIGVVTI

SEQ ID NO: 106 - DNA, 459 bp, Escherichia coli; identical to SEQ ID NO: 5
  source 1..459: mol_type = genomic DNA

SEQ ID NO: 107 - protein, 152 aa, Escherichia coli [sequence omitted]

SEQ ID NO: 108 - DNA, 741 bp, Escherichia coli
  source 1..741: mol_type = genomic DNA
atgtcagttattggtcgcattcactcctttgaatcctgtggaaccgtagacggcccaggtattcgctttatcacctttttccagggctgcctgatgcgctgcctgtattgtcataaccgcgacacctgggacacgcatggcggtaaagaagttaccgttgaagatttgatgaaggaagtggtgacctatcgccactttatgaacgcttccggcggcggcgttaccgcatccggcggtgaagcaatcctgcaagctgagtttgttcgtgactggttccgcgcctgcaaaaaagaaggcattcatacctgtctggacaccaacggttttgttcgtcgttacgatccggtgattgatgaactgctggaagtaaccgacctggtaatgctcgatctcaaacagatgaacgacgagatccaccaaaatctggttggagtttccaaccaccgcacgctggagttcgctaaatatctggcgaacaaaaatgtgaaggtgtggatccgctacgttgttgtcccaggctggtctgacgatgacgattcagcgcatcgcctcggtgaatttacccgtgatatgggcaacgttgagaaaatcgagcttctcccctaccacgagctgggcaaacacaaatgggtggcaatgggtgaagagtacaaactcgacggtgttaaaccaccgaagaaagagaccatggaacgcgtgaaaggcattcttgagcagtacggtcataaggtaatgttctaa

SEQ ID NO: 109 - protein, 246 aa, Escherichia coli
MSVIGRIHSFESCGTVDGPGIRFITFFQGCLMRCLYCHNRDTWDTHGGKEVTVEDLMKEVVTYRHFMNASGGGVTASGGEAILQAEFVRDWFRACKKEGIHTCLDTNGFVRRYDPVIDELLEVTDLVMLDLKQMNDEIHQNLVGVSNHRTLEFAKYLANKNVKVWIRYVVVPGWSDDDDSAHRLGEFTRDMGNVEKIELLPYHELGKHKWVAMGEEYKLDGVKPPKKETMERVKGILEQYGHKVMF

SEQ ID NO: 110 - DNA, 2283 bp, Escherichia coli
  source 1..2283: mol_type = genomic DNA
atgtccgagcttaatgaaaagttagccacagcctgggaaggttttaccaaaggtgactggcagaatgaagtaaacgtccgtgacttcattcagaaaaactacactccgtacgagggtgacgagtccttcctggctggcgctactgaagcgaccaccaccctgtgggacaaagtaatggaaggcgttaaactggaaaaccgcactcacgcgccagttgactttgacaccgctgttgcttccaccatcacctctcacgacgctggctacatcaacaagcagcttgagaaaatcgttggtctgcagactgaagctccgctgaaacgtgctcttatcccgttcggtggtatcaaaatgatcgaaggttcctgcaaagcgtacaaccgcgaactggatccgatgatcaaaaaaatcttcactgaataccgtaaaactcacaaccagggcgtgttcgacgtttacactccggacatcctgcgttgccgtaaatctggtgttctgaccggtctgccagatgcatatggccgtggccgtatcatcggtgactaccgtcgcgttgcgctgtacggtatcgactacctgatgaaagacaaactggcacagttcacttctctgcaggctgatctggaaaacggcgtaaacctggaacagactatccgtctgcgcgaagaaatcgctgaacagcaccgcgctctgggtcagatgaaagaaatggctgcgaaatacggctacgacatctctggtccggctaccaacgctcaggaagctatccagtggacttacttcggctacctggctgctgttaagtctcagaacggtgctgcaatgtccttcggtcgtacctccaccttcctggatgtgtacatcgaacgtgacctgaaagctggcaagatcaccgaacaagaagcgcaggaaatggttgaccacctggtcatgaaactgcgtatggttcgcttcctgcgtactccggaatacgatgaactgttctctggcgacccgatctgggcaaccgaatctatcggtggtatgggcctcgacggtcgtaccctggttaccaaaaacagcttccgtttcctgaacaccctgtacaccatgggtccgtctccggaaccgaacatgaccattctgtggtctgaaaaactgccgctgaacttcaagaaattcgccgctaaagtgtccatcgacacctcttctctgcagtatgagaacgatgacctgatgcgtccggacttcaacaacgatgactacgctattgcttgctgcgtaagcccgatgatcgttggtaaacaaatgcagttcttcggtgcgcgtgcaaacctggcgaaaaccatgctgtacgcaatcaacggcggcgttgacgaaaaactgaaaatgcaggttggtccgaagtctgaaccgatcaaaggcgatgtcctgaactatgatgaagtgatggagcgcatggatcacttcatggactggctggctaaacagtacatcactgcactgaacatcatccactacatgcacgacaagtacagctacgaagcctctctgatggcgctgcacgaccgtgacgttatccgcaccatggcgtgtggtatcgctggtctgtccgttgctgctgactccctgtctgcaatcaaatatgcgaaagttaaaccgattcgtgacgaagacggtctggctatcgacttcgaaatcgaaggcgaatacccgcagtttggtaacaatgatccgcgtgtagatgacctggctgttgacctggtagaacgtttcatgaagaaaattcagaaactgcacacctaccgtgacgctatcccgactcagtctgttctgaccatcacttctaacgttgtgtatggtaagaaaacgggtaacaccccagacggtcgtcgtgctggcgcgccgttcggaccgggtgctaacccgatgcacggtcgtgaccagaaaggtgcagtagcctctctgacttccgttgctaaactgccgtttgcttacgctaaagatggtatctcctacaccttctctatcgttccgaacgcactgggtaaagacgacgaagttcgtaagaccaacctggctggtctgatggatggttacttccaccacgaagcatccatcgaaggtggtcagcacctgaacgttaacgtgatgaaccgtgaaatgctgctcgacgcgatggaaaacccggaaaaatatccgcagctgaccatccgtgtatctggctacgcagtacgtttcaactcgctgactaaagaacagcagcaggacgttattactcgtaccttcactcaatctatgtaa

SEQ ID NO: 111 - protein, 760 aa, Escherichia coli
MSELNEKLATAWEGFTKGDWQNEVNVRDFIQKNYTPYEGDESFLAGATEATTTLWDKVMEGVKLENRTHAPVDFDTAVASTITSHDAGYINKQLEKIVGLQTEAPLKRALIPFGGIKMIEGSCKAYNRELDPMIKKIFTEYRKTHNQGVFDVYTPDILRCRKSGVLTGLPDAYGRGRIIGDYRRVALYGIDYLMKDKLAQFTSLQADLENGVNLEQTIRLREEIAEQHRALGQMKEMAAKYGYDISGPATNAQEAIQWTYFGYLAAVKSQNGAAMSFGRTSTFLDVYIERDLKAGKITEQEAQEMVDHLVMKLRMVRFLRTPEYDELFSGDPIWATESIGGMGLDGRTLVTKNSFRFLNTLYTMGPSPEPNMTILWSEKLPLNFKKFAAKVSIDTSSLQYENDDLMRPDFNNDDYAIACCVSPMIVGKQMQFFGARANLAKTMLYAINGGVDEKLKMQVGPKSEPIKGDVLNYDEVMERMDHFMDWLAKQYITALNIIHYMHDKYSYEASLMALHDRDVIRTMACGIAGLSVAADSLSAIKYAKVKPIRDEDGLAIDFEIEGEYPQFGNNDPRVDDLAVDLVERFMKKIQKLHTYRDAIPTQSVLTITSNVVYGKKTGNTPDGRRAGAPFGPGANPMHGRDQKGAVASLTSVAKLPFAYAKDGISYTFSIVPNALGKDDEVRKTNLAGLMDGYFHHEASIEGGQHLNVNVMNREMLLDAMENPEKYPQLTIRVSGYAVRFNSLTKEQQQDVITRTFTQSM

SEQ ID NO: 112 - DNA, 1476 bp, Escherichia coli
  source 1..1476: mol_type = genomic DNA
atggcggtaacgcaaacagcccaggcctgtgacctggtcattttcggcgcgaaaggcgaccttgcgcgtcgtaaattgctgccttccctgtatcaactggaaaaagccggtcagctcaacccggacacccggattatcggcgtagggcgtgctgactgggataaagcggcatataccaaagttgtccgcgaggcgctcgaaactttcatgaaagaaaccattgatgaaggtttatgggacaccctgagtgcacgtctggatttttgtaatctcgatgtcaatgacactgctgcattcagccgtctcggcgcgatgctggatcaaaaaaatcgtatcaccattaactactttgccatgccgcccagcacttttggcgcaatttgcaaagggcttggcgaggcaaaactgaatgctaaaccggcacgcgtagtcatggagaaaccgctggggacgtcgctggcgacctcgcaggaaatcaatgatcaggttggcgaatacttcgaggagtgccaggtttaccgtatcgaccactatcttggtaaagaaacggtgctgaacctgttggcgctgcgttttgctaactccctgtttgtgaataactgggacaatcgcaccattgatcatgttgagattaccgtggcagaagaagtggggatcgaagggcgctggggctattttgataaagccggtcagatgcgcgacatgatccagaaccacctgctgcaaattctttgcatgattgcgatgtctccgccgtctgacctgagcgcagacagcatccgcgatgaaaaagtgaaagtactgaagtctctgcgccgcatcgaccgctccaacgtacgcgaaaaaaccgtacgcgggcaatatactgcgggcttcgcccagggcaaaaaagtgccgggatatctggaagaagagggcgcgaacaagagcagcaatacagaaactttcgtggcgatccgcgtcgacattgataactggcgctgggccggtgtgccattctacctgcgtactggtaaacgtctgccgaccaaatgttctgaagtcgtggtctatttcaaaacacctgaactgaatctgtttaaagaatcgtggcaggatctgccgcagaataaactgactatccgtctgcaacctgatgaaggcgtggatatccaggtactgaataaagttcctggccttgaccacaaacataacctgcaaatcaccaagctggatctgagctattcagaaacctttaatcagacgcatctggcggatgcctatgaacgtttgctgctggaaaccatgcgtggtattcaggcactgtttgtacgtcgcgacgaagtggaagaagcctggaaatgggtagactccattactgaggcgtgggcgatggacaatgatgcgccgaaaccgtatcaggccggaacctggggacccgttgcctcggtggcgatgattacccgtgatggtcgttcctggaatgagtttgagtaa

SEQ ID NO: 113 - protein, 491 aa, Escherichia coli
MAVTQTAQACDLVIFGAKGDLARRKLLPSLYQLEKAGQLNPDTRIIGVGRADWDKAAYTKVVREALETFMKETIDEGLWDTLSARLDFCNLDVNDTAAFSRLGAMLDQKNRITINYFAMPPSTFGAICKGLGEAKLNAKPARVVMEKPLGTSLATSQEINDQVGEYFEECQVYRIDHYLGKETVLNLLALRFANSLFVNNWDNRTIDHVEITVAEEVGIEGRWGYFDKAGQMRDMIQNHLLQILCMIAMSPPSDLSADSIRDEKVKVLKSLRRIDRSNVREKTVRGQYTAGFAQGKKVPGYLEEEGANKSSNTETFVAIRVDIDNWRWAGVPFYLRTGKRLPTKCSEVVVYFKTPELNLFKESWQDLPQNKLTIRLQPDEGVDIQVLNKVPGLDHKHNLQITKLDLSYSETFNQTHLADAYERLLLETMRGIQALFVRRDEVEEAWKWVDSITEAWAMDNDAPKPYQAGTWGPVASVAMITRDGRSWNEFE

SEQ ID NO: 114 - DNA, 1812 bp, Escherichia coli
  source 1..1812: mol_type = genomic DNA
atgaatccacaattgttacgcgtaacaaatcgaatcattgaacgttcgcgcgagactcgctctgcttatctcgcccggatagaacaagcgaaaacttcgaccgttcatcgttcgcagttggcatgcggtaacctggcacacggtttcgctgcctgccagccagaagacaaagcctctttgaaaagcatgttgcgtaacaatatcgccatcatcacctcctataacgacatgctctccgcgcaccagccttatgaacactatccagaaatcattcgtaaagccctgcatgaagcgaatgcggttggtcaggttgcgggcggtgttccggcgatgtgtgatggtgtcacccaggggcaggatggaatggaattgtcgctgctaagccgcgaagtgatagcgatgtctgcggcggtggggctgtcccataacatgtttgatggtgctctgttcctcggtgtgtgcgacaagattgtcccgggtctgacgatggcagccctgtcgtttggtcatttgcctgcggtgtttgtgccgtctggaccgatggcaagcggtttgccaaataaagaaaaagtgcgtattcgccagctttatgccgaaggtaaagtggaccgcatggccttactggagtcagaagccgcgtcttaccatgcgccgggaacatgtactttctacggtactgccaacaccaaccagatggtggtggagtttatggggatgcagttgccaggctcttcttttgttcatccggattctccgctgcgcgatgctttgaccgccgcagctgcgcgtcaggttacacgcatgaccggtaatggtaatgaatggatgccgatcggtaagatgatcgatgagaaagtggtggtgaacggtatcgttgcactgctggcgaccggtggttccactaaccacaccatgcacctggtggcgatggcgcgcgcggccggtattcagattaactgggatgacttctctgacctttctgatgttgtaccgctgatggcacgtctctacccgaacggtccggccgatattaaccacttccaggcggcaggtggcgtaccggttctggtgcgtgaactgctcaaagcaggcctgctgcatgaagatgtcaatacggtggcaggttttggtctgtctcgttatacccttgaaccatggctgaataatggtgaactggactggcgggaaggggcggaaaaatcactcgacagcaatgtgatcgcttccttcgaacaacctttctctcatcatggtgggacaaaagtgttaagcggtaacctgggccgtgcggttatgaaaacctctgccgtgccggttgagaaccaggtgattgaagcgccagcggttgtttttgaaagccagcatgacgttatgccggcctttgaagcgggtttgctggaccgcgattgtgtcgttgttgtccgtcatcaggggccaaaagcgaacggaatgccagaattacataaactcatgccgccacttggtgtattattggaccggtgtttcaaaattgcgttagttaccgatggacgactctccggcgcttcaggtaaagtgccgtcagctatccacgtaacaccagaagcctacgatggcgggctgctggcaaaagtgcgcgacggggacatcattcgtgtgaatggacagacaggcgaactgacgctgctggtagacgaagcggaactggctgctcgcgaaccgcacattcctgacctgagcgcgtcacgcgtgggaacaggacgtgaattattcagcgccttgcgtgaaaaactgtccggtgccgaacagggcgcaacctgtatcactttttaa

SEQ ID NO: 115 - protein, 603 aa, Escherichia coli
MNPQLLRVTNRIIERSRETRSAYLARIEQAKTSTVHRSQLACGNLAHGFAACQPEDKASLKSMLRNNIAIITSYNDMLSAHQPYEHYPEIIRKALHEANAVGQVAGGVPAMCDGVTQGQDGMELSLLSREVIAMSAAVGLSHNMFDGALFLGVCDKIVPGLTMAALSFGHLPAVFVPSGPMASGLPNKEKVRIRQLYAEGKVDRMALLESEAASYHAPGTCTFYGTANTNQMVVEFMGMQLPGSSFVHPDSPLRDALTAAAARQVTRMTGNGNEWMPIGKMIDEKVVVNGIVALLATGGSTNHTMHLVAMARAAGIQINWDDFSDLSDVVPLMARLYPNGPADINHFQAAGGVPVLVRELLKAGLLHEDVNTVAGFGLSRYTLEPWLNNGELDWREGAEKSLDSNVIASFEQPFSHHGGTKVLSGNLGRAVMKTSAVPVENQVIEAPAVVFESQHDVMPAFEAGLLDRDCVVVVRHQGPKANGMPELHKLMPPLGVLLDRCFKIALVTDGRLSGASGKVPSAIHVTPEAYDGGLLAKVRDGDIIRVNGQTGELTLLVDEAELAAREPHIPDLSASRVGTGRELFSALREKLSGAEQGATCITF

SEQ ID NO: 116 - DNA, 642 bp, Escherichia coli
  source 1..642: mol_type = genomic DNA
atgaaaaactggaaaacaagtgcagaatcaatcctgaccaccggcccggttgtaccggttatcgtggtaaaaaaactggaacacgcggtgccgatggcaaaagcgttggttgctggtggggtgcgcgttctggaagtgactctgcgtaccgagtgtgcagttgacgctatccgtgctatcgccaaagaagtgcctgaagcgattgtgggtgccggtacggtgctgaatccacagcagctggcagaagtcactgaagcgggtgcacagttcgcaattagcccgggtctgaccgagccgctgctgaaagctgctaccgaagggactattcctctgattccggggatcagcactgtttccgaactgatgctgggtatggactacggtttgaaagagttcaaattcttcccggctgaagctaacggcggcgtgaaagccctgcaggcgatcgcgggtccgttctcccaggtccgtttctgcccgacgggtggtatttctccggctaactaccgtgactacctggcgctgaaaagcgtgctgtgcatcggtggttcctggctggttccggcagatgcgctggaagcgggcgattacgaccgcattactaagctggcgcgtgaagctgtagaaggcgctaagctgtaa

SEQ ID NO: 117 - protein, 213 aa, Escherichia coli
MKNWKTSAESILTTGPVVPVIVVKKLEHAVPMAKALVAGGVRVLEVTLRTECAVDAIRAIAKEVPEAIVGAGTVLNPQQLAEVTEAGAQFAISPGLTEPLLKAATEGTIPLIPGISTVSELMLGMDYGLKEFKFFPAEANGGVKALQAIAGPFSQVRFCPTGGISPANYRDYLALKSVLCIGGSWLVPADALEAGDYDRITKLAREAVEGAKL

SEQ ID NO: 118 - DNA, 1407 bp, Escherichia coli
  source 1..1407: mol_type = genomic DNA
atgtccaagcaacagatcggcgtagtcggtatggcagtgatgggacgcaaccttgcgctcaacatcgaaagccgtggttataccgtctctattttcaaccgttcccgtgagaagacggaagaagtgattgccgaaaatccaggcaagaaactggttccttactatacggtgaaagagtttgtcgaatctctggaaacgcctcgtcgcatcctgttaatggtgaaagcaggtgcaggcacggatgctgctattgattccctcaaaccatatctcgataaaggagacatcatcattgatggtggtaacaccttcttccaggacactattcgtcgtaatcgtgagctttcagcagagggctttaacttcatcggtaccggtgtttctggcggtgaagagggggcgctgaaaggtccttctattatgcctggtggccagaaagaagcctatgaattggtagcaccgatcctgaccaaaatcgccgccgtagctgaagacggtgaaccatgcgttacctatattggtgccgatggcgcaggtcactatgtgaagatggttcacaacggtattgaatacggcgatatgcagctgattgctgaagcctattctctgcttaaaggtggcctgaacctcaccaacgaagaactggcgcagacctttaccgagtggaataacggtgaactgagcagttacctgatcgacatcaccaaagatatcttcaccaaaaaagatgaagacggtaactacctggttgatgtgatcctggatgaagcggctaacaaaggtaccggtaaatggaccagccagagcgcgctggatctcggcgaaccgctgtcgctgattaccgagtctgtgtttgcacgttatatctcttctctgaaagatcagcgtgttgccgcatctaaagttctctctggtccgcaagcacagccagcaggcgacaaggctgagttcatcgaaaaagttcgtcgtgcgctgtatctgggcaaaatcgtttcttacgcccagggcttctctcagctgcgtgctgcgtctgaagagtacaactgggatctgaactacggcgaaatcgcgaagattttccgtgctggctgcatcatccgtgcgcagttcctgcagaaaatcaccgatgcttatgccgaaaatccacagatcgctaacctgttgctggctccgtacttcaagcaaattgccgatgactaccagcaggcgctgcgtgatgtcgttgcttatgcagtacagaacggtattccggttccgaccttctccgcagcggttgcctattacgacagctaccgtgctgctgttctgcctgcgaacctgatccaggcacagcgtgactattttggtgcgcatacttataagcgtattgataaagaaggtgtgttccataccgaatggctggattaa

SEQ ID NO: 119 - protein, 468 aa, Escherichia coli
MSKQQIGVVGMAVMGRNLALNIESRGYTVSIFNRSREKTEEVIAENPGKKLVPYYTVKEFVESLETPRRILLMVKAGAGTDAAIDSLKPYLDKGDIIIDGGNTFFQDTIRRNRELSAEGFNFIGTGVSGGEEGALKGPSIMPGGQKEAYELVAPILTKIAAVAEDGEPCVTYIGADGAGHYVKMVHNGIEYGDMQLIAEAYSLLKGGLNLTNEELAQTFTEWNNGELSSYLIDITKDIFTKKDEDGNYLVDVILDEAANKGTGKWTSQSALDLGEPLSLITESVFARYISSLKDQRVAASKVLSGPQAQPAGDKAEFIEKVRRALYLGKIVSYAQGFSQLRAASEEYNWDLNYGEIAKIFRAGCIIRAQFLQKITDAYAENPQIANLLLAPYFKQIADDYQQALRDVVAYAVQNGIPVPTFSAAVAYYDSYRAAVLPANLIQAQRDYFGAHTYKRIDKEGVFHTEWLD

SEQ ID NO: 120 - DNA, 1533 bp, Escherichia coli
  source 1..1533: mol_type = genomic DNA
atgcgaattggcataccaagagaacggttaaccaatgaaacccgtgttgcagcaacgccaaaaacagtggaacagctgctgaaactgggttttaccgtcgcggtagagagcggcgcgggtcaactggcaagttttgacgataaagcgtttgtgcaagcgggcgctgaaattgtagaagggaatagcgtctggcagtcagagatcattctgaaggtcaatgcgccgttagatgatgaaattgcgttactgaatcctgggacaacgctggtgagttttatctggcctgcgcagaatccggaattaatgcaaaaacttgcggaacgtaacgtgaccgtgatggcgatggactctgtgccgcgtatctcacgcgcacaatcgctggacgcactaagctcgatggcgaacatcgccggttatcgcgccattgttgaagcggcacatgaatttgggcgcttctttaccgggcaaattactgcggccgggaaagtgccaccggcaaaagtgatggtgattggtgcgggtgttgcaggtctggccgccattggcgcagcaaacagtctcggcgcgattgtgcgtgcattcgacacccgcccggaagtgaaagaacaagttcaaagtatgggcgcggaattcctcgagctggattttaaagaggaagctggcagcggcgatggctatgccaaagtgatgtcggacgcgttcatcaaagcggaaatggaactctttgccgcccaggcaaaagaggtcgatatcattgtcaccaccgcgcttattccaggcaaaccagcgccgaagctaattacccgtgaaatggttgactccatgaaggcgggcagtgtgattgtcgacctggcagcccaaaacggcggcaactgtgaatacaccgtgccgggtgaaatcttcactacggaaaatggtgtcaaagtgattggttataccgatcttccgggccgtctgccgacgcaatcctcacagctttacggcacaaacctcgttaatctgctgaaactgttgtgcaaagagaaagacggcaatatcactgttgattttgatgatgtggtgattcgcggcgtgaccgtgatccgtgcgggcgaaattacctggccggcaccgccgattcaggtatcagctcagccgcaggcggcacaaaaagcggcaccggaagtgaaaactgaggaaaaatgtacctgctcaccgtggcgtaaatacgcgttgatggcgctggcaatcattctttttggctggatggcaagcgttgcgccgaaagaattccttgggcacttcaccgttttcgcgctggcctgcgttgtcggttattacgtggtgtggaatgtatcgcacgcgctgcatacaccgttgatgtcggtcaccaacgcgatttcagggattattgttgtcggagcactgttgcagattggccagggcggctgggttagcttccttagttttatcgcggtgcttatagccagcattaatattttcggtggcttcaccgtgactcagcgcatgctgaaaatgttccgcaaaaattaa

SEQ ID NO: 121 - protein, 510 aa, Escherichia coli
MRIGIPRERLTNETRVAATPKTVEQLLKLGFTVAVESGAGQLASFDDKAFVQAGAEIVEGNSVWQSEIILKVNAPLDDEIALLNPGTTLVSFIWPAQNPELMQKLAERNVTVMAMDSVPRISRAQSLDALSSMANIAGYRAIVEAAHEFGRFFTGQITAAGKVPPAKVMVIGAGVAGLAAIGAANSLGAIVRAFDTRPEVKEQVQSMGAEFLELDFKEEAGSGDGYAKVMSDAFIKAEMELFAAQAKEVDIIVTTALIPGKPAPKLITREMVDSMKAGSVIVDLAAQNGGNCEYTVPGEIFTTENGVKVIGYTDLPGRLPTQSSQLYGTNLVNLLKLLCKEKDGNITVDFDDVVIRGVTVIRAGEITWPAPPIQVSAQPQAAQKAAPEVKTEEKCTCSPWRKYALMALAIILFGWMASVAPKEFLGHFTVFALACVVGYYVVWNVSHALHTPLMSVTNAISGIIVVGALLQIGQGGWVSFLSFIAVLIASINIFGGFTVTQRMLKMFRKN

SEQ ID NO: 122 - DNA, 1389 bp, Escherichia coli
  source 1..1389: mol_type = genomic DNA
atgtctggaggattagttacagctgcatacattgttgccgcgatcctgtttatcttcagtctggccggtctttcgaaacatgaaacgtctcgccagggtaacaacttcggtatcgccgggatggcgattgcgttaatcgcaaccatttttggaccggatacgggtaatgttggctggatcttgctggcgatggtcattggtggggcaattggtatccgtctggcgaagaaagttgaaatgaccgaaatgccagaactggtggcgatcctgcatagcttcgtgggtctggcggcagtgctggttggctttaacagctatctgcatcatgacgcgggaatggcaccgattctggtcaatattcacctgacggaagtgttcctcggtatcttcatcggggcggtaacgttcacgggttcggtggtggcgttcggcaaactgtgtggcaagatttcgtctaaaccattgatgctgccaaaccgtcacaaaatgaacctggcggctctggtcgtttccttcctgctgctgattgtatttgttcgcacggacagcgtcggcctgcaagtgctggcattgctgataatgaccgcaattgcgctggtattcggctggcatttagtcgcctccatcggtggtgcagatatgccagtggtggtgtcgatgctgaactcgtactccggctgggcggctgcggctgcgggctttatgctcagcaacgacctgctgattgtgaccggtgcgctggtcggttcttcgggggctatcctttcttacattatgtgtaaggcgatgaaccgttcctttatcagcgttattgcgggtggtttcggcaccgacggctcttctactggcgatgatcaggaagtgggtgagcaccgcgaaatcaccgcagaagagacagcggaactgctgaaaaactcccattcagtgatcattactccggggtacggcatggcagtcgcgcaggcgcaatatcctgtcgctgaaattactgagaaattgcgcgctcgtggtattaatgtgcgtttcggtatccacccggtcgcggggcgtttgcctggacatatgaacgtattgctggctgaagcaaaagtaccgtatgacatcgtgctggaaatggacgagatcaatgatgactttgctgataccgataccgtactggtgattggtgctaacgatacggttaacccggcggcgcaggatgatccgaagagtccgattgctggtatgcctgtgctggaagtgtggaaagcgcagaacgtgattgtctttaaacgttcgatgaacactggctatgctggtgtgcaaaacccgctgttcttcaaggaaaacacccacatgctgtttggtgacgccaaagccagcgtggatgcaatcctgaaagctctgtaa

SEQ ID NO: 123 - protein, 462 aa, Escherichia coli
MSGGLVTAAYIVAAILFIFSLAGLSKHETSRQGNNFGIAGMAIALIATIFGPDTGNVGWILLAMVIGGAIGIRLAKKVEMTEMPELVAILHSFVGLAAVLVGFNSYLHHDAGMAPILVNIHLTEVFLGIFIGAVTFTGSVVAFGKLCGKISSKPLMLPNRHKMNLAALVVSFLLLIVFVRTDSVGLQVLALLIMTAIALVFGWHLVASIGGADMPVVVSMLNSYSGWAAAAAGFMLSNDLLIVTGALVGSSGAILSYIMCKAMNRSFISVIAGGFGTDGSSTGDDQEVGEHREITAEETAELLKNSHSVIITPGYGMAVAQAQYPVAEITEKLRARGINVRFGIHPVAGRLPGHMNVLLAEAKVPYDIVLEMDEINDDFADTDTVLVIGANDTVNPAAQDDPKSPIAGMPVLEVWKAQNVIVFKRSMNTGYAGVQNPLFFKENTHMLFGDAKASVDAILKAL

SEQ ID NO: 124 - DNA, 1014 bp, Streptococcus mutans
  source 1..1014: mol_type = genomic DNA
atggtagttaaagttggtattaacggttttggtcgtattggacgtcttgctttccgtcgtatccaaaacgtcgaaggtgttgaagttacacgtatcaatgaccttacagatccaaacatgcttgcacacttgttgaaatatgattcaactcaaggtcgttttgacggtaatgtcgaagttaaagaaggcggatttgaagttaatggtaaattcgttaaggtttcagctgaacgtgatccagaacagattgactgggctgctgatggtgtagaaatcgttcttgaagcaactggcttctttgcaagcaaagcagctgctgaaaaacacttacatgctaatggtggagctaaaaaggttgttatcactgcaccaggtggtaatgatattaagacaatcgtctttaacaccaaccacgatgttcttgatggtactgaaacagttatttcaggtgcttcatgtactacaaactgtcttgcaccaatggcgaaagctttacatgataactttagtatcaaagaaggtttgatgacaactatccatgcatacactggtgatcaaatggttcttgatggaccacaccgtaaaggcgaccttcgtcgtgcacgtgctgctgcagcaaacatcgttcctaactcaactggtgctgctaaagctattggtcttgttattcctgaattgaatggtaaacttgacggtgctgcacaacgtgttccagttccaacaggttcagttactgaattagttgctgtacttgataaaaaggtaactgttgatgaagtgaatgctgctatgaaagctgctgcaaatgaatcttacggatatactgaagatccaatcgtatcatcagatattgtaggtatgtcttttggttcattgtttgatgcaactcaaacaaaagttcttgatgttgatggcaagcaattggttaaagttgtttcatggtatgataacgaaatgtcatatacttcacaacttgtacgtactcttgagtactttgcaaaaatcgctaaataa

SEQ ID NO: 125 - protein, 337 aa, Streptococcus mutans
MVVKVGINGFGRIGRLAFRRIQNVEGVEVTRINDLTDPNMLAHLLKYDSTQGRFDGNVEVKEGGFEVNGKFVKVSAERDPEQIDWAADGVEIVLEATGFFASKAAAEKHLHANGGAKKVVITAPGGNDIKTIVFNTNHDVLDGTETVISGASCTTNCLAPMAKALHDNFSIKEGLMTTIHAYTGDQMVLDGPHRKGDLRRARAAAANIVPNSTGAAKAIGLVIPELNGKLDGAAQRVPVPTGSVTELVAVLDKKVTVDEVNAAMKAAANESYGYTEDPIVSSDIVGMSFGSLFDATQTKVLDVDGKQLVKVVSWYDNEMSYTSQLVRTLEYFAKIAK

SEQ ID NO: 126 - DNA, 1005 bp, Clostridium acetobutylicum
  source 1..1005: mol_type = genomic DNA
atggcaaagatagctattaatggttttggaagaataggaagattagctttaagaagaattcttgaagtacctggattggaagttgttgcaataaacgacttaactgatgcaaaaatgttagcacacttatttaaatatgattcatcacaaggaagattcaatggagaaattgaagttaaagaaggagctttcgtagtaaacggaaaagaagttaaagttttcgctgaagcagatcctgaaaaattaccttggggagatcttggaatagacgttgttcttgagtgcacaggtttcttcacaaagaaagaaaaagcagaagctcacgtaagagcaggcgctaaaaaagttgttatatcagctccagctggaaacgacttaaagacaatagttttcaacgttaataatgaagatcttgatggaacagaaacagttatatcaggtgcatcatgcacaactaactgcttagctccaatggctaaagtattaaatgataaatttggaatagaaaaaggattcatgactacaattcatgcgttcactaatgaccaaaacacattagatggtccacacagaaaaggagatttaagaagagctagagctgctgctgtaagtatcatccctaactcaactggtgctgctaaagctataagccaagttattcctgacttagctggaaaattagacggaaacgctcaaagagttccagttccaactggttcaataactgaattagtttcagttcttaagaaaaaagttacagttgaagaaatcaacgctgctatgaaagaagctgctgatgaatcatttggatacactgaagatccaatcgtttcagctgacgtagtaggaatcaactacggatcattatttgatgcaactttaactaaaattgttgatgttaacggatcacaattagttaaaacagctgcttggtatgataatgaaatgtcatacacttcacaattagttagaactttagcttactttgcaaaaatagcaaaatag

SEQ ID NO: 127 - protein, 334 aa, Clostridium acetobutylicum
MAKIAINGFGRIGRLALRRILEVPGLEVVAINDLTDAKMLAHLFKYDSSQGRFNGEIEVKEGAFVVNGKEVKVFAEADPEKLPWGDLGIDVVLECTGFFTKKEKAEAHVRAGAKKVVISAPAGNDLKTIVFNVNNEDLDGTETVISGASCTTNCLAPMAKVLNDKFGIEKGFMTTIHAFTNDQNTLDGPHRKGDLRRARAAAVSIIPNSTGAAKAISQVIPDLAGKLDGNAQRVPVPTGSITELVSVLKKKVTVEEINAAMKEAADESFGYTEDPIVSADVVGINYGSLFDATLTKIVDVNGSQLVKTAAWYDNEMSYTSQLVRTLAYFAKIAK

SEQ ID NO: 128 - DNA, 1017 bp, Pisum sativum
  source 1..1017: mol_type = genomic DNA
atgggtgccaagatcaagatcggaatcaacggattcggaagaatcggacgtttagttgctagagtagctttgaagagagatgatgttgaactcgttgcagttaacgatcctttcatcaccactgattacatgacgtatatgtttaagtacgacagtgttcacggacagtggaagaacgacgaactcaccgtcaaggactctaacactcttctcttcggtcagaagccagttactgtctttgcacacaggaacccagaagagatcccatgggccagcactggtgctgatatcattgttgagtctactggtgtttttactgataaggacaaggctgctgctcatttgaagggtggtgccaagaaggtcatcatttctgctcccagtaaagatgctcccatgtttgttgttggtgttaatgagaatgaatacaagccagagtttgacattatttccaatgctagctgcaccaccaactgccttgcaccacttgcaaaggttattaatgacaggtttggcattgttgagggtctcatgaccactgtccattccatcaccgccacccagaagactgttgatggaccatcaagcaaggactggagaggtggaagagctgcttcatttaacatcattcccagcagtaccggagctgctaaggctgtcggcaaagtgcttcctgctttgaatggaaagttgaccggtatgtcattccgtgtcccaactgtggatgtctccgttgttgaccttacagtgaggcttgagaaggccgccacctatgatgaaatcaaagctgctatcaaggaagagtctgagggcaagttgaaaggaatccttggttacactgaagacgatgtggtctccactgactttattggtgacaccaggtcaagtatctttgatgccaaggcaggaattgccttaaatgacaagtttgttaagcttgtctcatggtatgacaacgagttgggttacagtacccgtgtggttgacctcattgttcacattgctaaacaactttaa

SEQ ID NO: 129 - protein, 338 aa, Pisum sativum
MGAKIKIGINGFGRIGRLVARVALKRDDVELVAVNDPFITTDYMTYMFKYDSVHGQWKNDELTVKDSNTLLFGQKPVTVFAHRNPEEIPWASTGADIIVESTGVFTDKDKAAAHLKGGAKKVIISAPSKDAPMFVVGVNENEYKPEFDIISNASCTTNCLAPLAKVINDRFGIVEGLMTTVHSITATQKTVDGPSSKDWRGGRAASFNIIPSSTGAAKAVGKVLPALNGKLTGMSFRVPTVDVSVVDLTVRLEKAATYDEIKAAIKEESEGKLKGILGYTEDDVVSTDFIGDTRSSIFDAKAGIALNDKFVKLVSWYDNELGYSTRVVDLIVHIAKQL

(19) United States
(12) Patent Application Publication
(10) Pub. No.: US 2025/0340828 A1
(43) Pub. Date: Nov. 6, 2025

(54) MICROORGANISM AND METHOD FOR THE IMPROVED PRODUCTION OF LEUCINE AND/OR ISOLEUCINE

(71) Applicant: METABOLIC EXPLORER, SAINT BEAUZIRE (FR)

(72) Inventors: Thomas DESFOUGERES, DISSAY (FR); Céline RAYNAUD, SAINT BEAUZIRE (FR); Philippe SOUCAILLE, DEYME (FR); Perrine VASSEUR, SAINT-LAURE (FR)

(73) Assignee: METABOLIC EXPLORER, SAINT BEAUZIRE (FR)

(21) Appl. No.: 18/843,159

(22) PCT Filed: Mar. 1, 2023
(86) PCT No.: PCT/EP2023/055125

§ 371 Date: Aug. 30, 2024

(30) Foreign Application Priority Data
Mar. 1, 2022 (EP) 22305234.1

(51) Int. Cl. C12N 1/20; C12N 9/02; C12R 1/19

(52) U.S. Cl. CPC C12N 1/20; C12N 9/0008; C12R 2001/19; C12Y 102/01012

(57) ABSTRACT

The present invention relates to a microorganism genetically modified for the production of leucine and/or isoleucine, wherein said microorganism comprises the expression of a heterologous gapN gene coding an NADP-dependent glyceralde-O hyde-3-phosphate dehydrogenase, and the attenuation of the expression of gapA and gltA genes as compared to an unmodified microorganism. The present invention also relates to a method for the production of leucine and/or isoleucine using said microorganism.

### REFERENCE TO ELECTRONIC SEQUENCE LISTING

The application contains a Sequence Listing which has been submitted electronically in .XML format and is hereby incorporated by reference in its entirety. Said .XML copy, created on Nov. 18, 2024, is named “3493-1010PUS1.xml” and is 220,813 bytes in size. The sequence listing contained in this .XML file is part of the specification and is hereby incorporated by reference herein in its entirety.

### FIELD OF THE INVENTION

The present invention relates to a microorganism genetically modified for the improved production of leucine and/or isoleucine and to a method for the improved production of leucine and/or isoleucine using said microorganism.

### BACKGROUND OF THE INVENTION

Amino acids are used in many industrial fields, including the food, animal feed, cosmetics, pharmaceutical, and chemical industries and have an annual worldwide market growth rate of an estimated 5 to 7% (Leuchtenberger, et al., 2005). Among these, leucine and isoleucine are particularly important for the nutrition of humans and a number of livestock species as they are essential amino acids that cannot be synthesized in mammals. As such, they are commonly used as food additives and in dietary supplements, with leucine also being used as a flavor enhancer. Leucine and isoleucine also notably function as precursors in the synthesis of antibiotics, such as polyketides.

Leucine and isoleucine may be produced via chemical synthesis, extraction from protein hydrolysates, or microbial fermentation. Of these techniques, fermentation is the most commonly used today, due to the associated economic and environmental advantages. In particular, fermentation provides a useful way of using abundant, renewable, and/or inexpensive materials as the main source of carbon. Furthermore, while both D- and L-enantiomers are generated in equimolar amounts when using chemical synthesis, requiring additional downstream isolation of the L-enantiomer, fermentation produces only the L-enantiomer. Biosynthesis of leucine and isoleucine by fermentation is generally performed using microorganisms of the Corynebacterium or Escherichia genera, such as Corynebacterium glutamicum or Escherichia coli.

Originally, leucine and isoleucine producing strains were isolated by random mutagenesis. However, more recently, microorganisms have been subject to rational metabolic engineering, with strategies to improve amino acid production focusing mainly on removing feedback inhibition, modifying upstream central carbon flux, and reducing downstream synthesis of undesired by-products (see e.g., Yamamato et al., 2017, Park et al., 2010).

As an example, amino acid production may be improved by incorporation of feedback-resistant threonine dehydratase and aspartate kinase Ill (encoded by ilvA and lysC, respectively, in E. coli) for isoleucine, while removal of feedback inhibition of leuA may improve leucine production. As a further example, production may be improved by overexpressing the leuE gene encoding an L-leucine specific exporter or deleting the livK gene encoding an L-leucine specific transporter in E. coli (Park et al., 2010).

In view of the ever-increasing demand for leucine and isoleucine in industrial applications, there remains a need for further improvements in the production of these amino acids. In particular, there remains a need for improved microorganisms that are able to produce leucine or isoleucine with high levels of productivity, titer, and yield, in particular from an inexpensive and/or abundant carbon source such as glucose. There also remains a need for improved methods for the production of leucine or isoleucine on an industrial scale, ideally wherein the productivity, titer, and yield of leucine or isoleucine is at least similar to that obtained with current methods.

### BRIEF DESCRIPTION OF THE INVENTION

The present invention addresses the above needs, providing a microorganism genetically modified for the production of leucine and/or isoleucine and methods for the production of leucine and/or isoleucine using said microorganism. The microorganism genetically modified for the production of leucine and/or isoleucine notably expresses a heterologous gapN gene coding an NADP-dependent glyceraldehyde-3-phosphate dehydrogenase and has attenuated expression of gapA coding glyceraldehyde-3-phosphate dehydrogenase A and gltA coding citrate synthase as compared to an unmodified microorganism. Indeed, the inventors have found that by such a microorganism advantageously shows improved production of leucine or isoleucine as productivity, titer and yield are increased.

Preferably, the gapN gene codes an NADP-dependent glyceraldehyde-3-phosphate dehydrogenase having at least 80% identity with GapN from Streptococcus mutans.

Preferably, the gapA gene is deleted.

Preferably, the microorganism further comprises an attenuation of the expression of the gapB and/or gapC genes as compared to an unmodified microorganism, preferably a deletion of the gapB and gapC genes.

Preferably, the microorganism further comprises an overexpression of at least one gene selected from among ackA, pta, and acs, as compared to an unmodified microorganism.

Preferably, the microorganism is genetically modified for the production of leucine and comprises an overexpression of the following genes: ilvBN, ilvC, ilvD, leuA*, leuB, leuC, leuD, and ilvE, as compared to an unmodified microorganism.

Preferably, the microorganism is genetically modified for the production of isoleucine and comprises:
a) the expression of a heterologous cimA* gene,
b) an overexpression of the following genes: ilvIH*, ilvC, ilvD, leuB, leuC, leuD, and ilvE, as compared to an unmodified microorganism, and
c) an attenuation of the leuA gene.

Preferably, the microorganism further comprises:
a) an attenuation of the expression of at least one gene selected from among udhA, aceEF, sucAB, poxB, brnQ, livKHMGF, adhE, IdhA, frdABCD, mgsA, pflAB, zwf, edd, eda, and gnd, and/or
b) an overexpression of at least one gene selected from among pntAB, gdhA, leuE, and ygaZH,
   as compared to an unmodified microorganism.

Preferably, in the microorganism, at least one gene selected from among udhA, aceEF, sucAB, poxB, brnQ, livKHMGF, adhE, IdhA, frdABCD, mgsA, pflAB, zwf, edd, eda, and gnd is deleted.

Preferably, the microorganism belongs to the Escherichia genus, more preferably wherein the microorganism is E. coli, the Corynebacterium genus, more preferably wherein the microorganism is C. glutamicum, or the Streptococcus genus, more preferably wherein the microorganism is chosen among S. thermophilus and S. salivarius, most preferably wherein the microorganism is E. coli.

The invention further relates to a method for the production of leucine and/or isoleucine comprising the steps of:
a) culturing a microorganism genetically modified for the production of leucine and/or isoleucine as provided herein in an appropriate culture medium comprising a source of carbon, and
b) recovering leucine and/or isoleucine from the culture medium.

Preferably, the culture medium further comprises acetate.

Preferably, the source of carbon is glucose, fructose, galactose, lactose, and/or sucrose.

Preferably step b) of the method comprises a step of crystallization.

### DETAILED DESCRIPTION

Before describing the present invention in detail, it is to be understood that the invention is not limited to particularly exemplified microorganism and/or methods and may, of course, vary. Indeed, various modifications, substitutions, omissions, and changes may be made without departing from the scope of the invention. It shall also be understood that the terminology used herein is for the purpose of describing particular embodiments of the invention only, and is not intended to be limiting.

All publications, patents and patent applications cited herein, whether supra or infra, are hereby incorporated by reference in their entirety. Furthermore, the practice of the present invention employs, unless otherwise indicated, conventional microbiological and molecular biological techniques that are within the skill of the art. Such techniques are well-known to the skilled person, and are fully explained in the literature (see e.g., Prescott et al. (1999) and Sambrook and Russell (2001)).

Unless defined otherwise, all technical and scientific terms used herein have the same meanings as are commonly understood by one of ordinary skill in the art to which this invention belongs. Although any materials and methods similar or equivalent to those described herein can be used to practice or test the present invention, preferred material and methods are provided.

It must be noted that as used herein and in the appended claims, the singular forms “a,” “an,” and “the,” include plural reference unless the context clearly dictates otherwise. Thus, for example, a reference to “a microorganism” includes a plurality of such microorganisms, and so forth.

The terms “comprise,” “contain,” “include,” and variations thereof such as “comprising” are used herein in an inclusive sense, i.e., to specify the presence of the stated features but not to preclude the presence or addition of further features in various embodiments of the invention.

A first aspect of the invention relates to a microorganism genetically modified for the production of leucine and/or isoleucine. The term “microorganism,” as used herein, refers to a living microscopic organism, which may be a single cell or a multicellular organism and which can generally be found in nature. The microorganism provided herein is preferably a bacterium.

Preferably, the microorganism is selected within the Enterobacteriaceae, Streptococcaceae, or Corynebacteriaceae family. More preferably, the microorganism is a species of the Escherichia, Streptococcus, or Corynebacterium genus. Even more preferably, said Enterobacteriaceae bacterium is Escherichia coli, said Streptococcaceae bacterium is Streptococcus thermophilus or Streptococcus salivarius, and said Corynebacteriaceae bacterium is Corynebacterium glutamicum. Most preferably, the microorganism is E. coli.

The terms “recombinant microorganism,” “genetically modified microorganism,” or “microorganism genetically modified” are used interchangeably herein and refer to a microorganism or a strain of microorganism that has been genetically modified or genetically engineered. This means, according to the usual meaning of these terms, that the microorganism of the invention is not found in nature and is genetically modified when compared to the “parental” microorganism from which it is derived. The “parental” microorganism may occur in nature (i.e., a wild-type microorganism) or may have been previously modified. The recombinant microorganism of the invention may notably be modified by the introduction, deletion, and/or modification of genetic elements. Such modifications can be performed, e.g., by genetic engineering or by adaptation, wherein a microorganism is cultured in conditions that apply a specific stress on the microorganism and induce mutagenesis and/or by forcing the development and evolution of metabolic pathways by combining directed mutagenesis and evolution under specific selection pressure.

A microorganism genetically modified for the increased production of leucine and/or isoleucine means that said microorganism is a recombinant microorganism that has increased production of leucine and/or isoleucine as compared to a parent microorganism which does not comprise the genetic modification. In other words, said microorganism has been genetically modified for increased production of leucine and/or isoleucine as compared to a corresponding unmodified microorganism.

A microorganism may notably be modified to modulate the expression level of an endogenous gene or the level of production of the corresponding protein or the activity of the corresponding enzyme. The term “endogenous gene” means that the gene was present in the microorganism before any genetic modification. Endogenous genes may be overexpressed by introducing heterologous sequences in addition to, or to replace, endogenous regulatory elements. Endogenous genes may also be overexpressed by introducing one or more supplementary copies of the gene into the chromosome or on a plasmid. In this case, the endogenous gene initially present in the microorganism may be deleted. Endogenous gene expression levels, protein production levels, or the activity of the encoded protein, can also be increased or attenuated by introducing mutations into the coding sequence of a gene or into non-coding sequences. These mutations may be synonymous, when no modification in the corresponding amino acid occurs, or non-synonymous, when the corresponding amino acid is altered. Synonymous mutations do not have any impact on the function of translated proteins, but may have an impact on the regulation of the corresponding genes or even of other genes, if the mutated sequence is located in a binding site for a regulator factor. Non-synonymous mutations may have an impact on the function or activity of the translated protein as well as on regulation, depending the nature of the mutated sequence.

In particular, mutations in non-coding sequences may be located upstream of the coding sequence (i.e., in the promoter region, in an enhancer, silencer, or insulator region, in a specific transcription factor binding site) or downstream of the coding sequence. Mutations introduced in the promoter region may be in the core promoter, proximal promoter or distal promoter. Mutations may be introduced by site-directed mutagenesis using, for example, Polymerase Chain Reaction (PCR), by random mutagenesis techniques for example via mutagenic agents (Ultra-Violet rays or chemical agents like nitrosoguanidine (NTG) or ethylmethanesulfonate (EMS)) or DNA shuffling or error-prone PCR or using culture conditions that apply a specific stress on the microorganism and induce mutagenesis. The insertion of one or more supplementary nucleotide(s) in the region located upstream of a gene can notably modulate gene expression.

A particular way of modulating endogenous gene expression is to exchange the endogenous promoter of a gene (e.g., wild-type promoter) with a stronger or weaker promoter to upregulate or downregulate expression of the endogenous gene. The promoter may be endogenous (i.e., originating from the same species) or exogenous (i.e., originating from a different species). It is well within the ability of the person skilled in the art to select an appropriate promoter for modulating the expression of an endogenous gene. Such a promoter be, for example, a Ptrc, Ptac, Ptet, or Plac promoter, or a lambda P_{L} (PL) or lambda P_{R} (PR) promoter. The promoter may be “inducible” by a particular compound or by specific external conditions, such as temperature or light or a small molecule, such as an antibiotic.

A particular way of modulating endogenous protein activity is to introduce nonsynonymous mutations in the coding sequence of the corresponding gene, e.g., according to any of the methods described above. A non-synonymous amino acid mutation that is present in a transcription factor may notably alter binding affinity of the transcription factor toward a cis-element, alter ligand binding to the transcription factor, etc.

A microorganism may also be genetically modified to express one or more exogenous or heterologous genes so as to overexpress the corresponding gene product (e.g., an enzyme). An “exogenous” or “heterologous” gene as used herein refers to a gene encoding a protein or polypeptide that is introduced into a microorganism in which said gene does not naturally occur. The gapN and cimA genes are notably heterologous genes in the context of the present invention. In particular, a heterologous gene may be directly integrated into the chromosome of the microorganism, or be expressed extra-chromosomally within the microorganism by plasmids or vectors. For successful expression, the heterologous gene(s) must be introduced into the microorganism with all of the regulatory elements necessary for their expression or be introduced into a microorganism that already comprises all of the regulatory elements necessary for their expression. The genetic modification or transformation of microorganisms with one or more exogenous genes is a routine task for those skilled in the art.

One or more copies of a given heterologous gene can be introduced on a chromosome by methods well-known in the art, such as by genetic recombination. When a gene is expressed extra-chromosomally, it can be carried by a plasmid or a vector. Different types of plasmid are notably available, which may differ in respect to their origin of replication and/or on their copy number in the cell. For example, a microorganism transformed by a plasmid can contain 1 to 5 copies of the plasmid, about 20 copies, or even up to 500 copies, depending on the nature of the selected plasmid. A variety of plasmids having different origins of replication and/or copy numbers are well-known in the art and can be easily selected by the skilled practitioner for such purposes, including, for example, pTrc, pACYC184, pBR322, pUC18, pUC19, pKC30, pRep4, pHS1, pHS2, pPLc236, or pCL1920.

It should be understood that, in the context of the present invention, when a heterologous gene encoding a protein of interest is expressed in a microorganism, such as E. coli, a synthetic version of this gene is preferably constructed by replacing non-preferred codons or less preferred codons with preferred codons of said microorganism which encode the same amino acid. Indeed, it is well-known in the art that codon usage varies between microorganism species, and that this may impact the recombinant production level of a protein of interest. To overcome this issue, codon optimization methods have been developed, and are extensively described by Graf et al. (2000), Deml et al. (2001) and Davis & Olsen (2011). Several software programs have notably been developed for codon optimization determination such as the GeneOptimizer® software (Lifetechnologies) or the OptimumGene™ software of (GenScript). In other words, the heterologous gene encoding a protein of interest is preferably codon-optimized for production in the chosen microorganism. As a particular example, the heterologous gapN gene may be codon optimized for expression in a microorganism such as E. coli.

On the basis of a given amino acid sequence, the skilled person is furthermore able to identify an appropriate polynucleotide coding for said polypeptide (e.g., in the available databases, such as Uniprot), or to synthesize the corresponding polypeptide or a polynucleotide coding for said polypeptide. De novo synthesis of a polynucleotide can be performed, for example, by initially synthesizing individual nucleic acid oligonucleotides and hybridizing these with oligonucleotides complementary thereto, such that they form a double-stranded DNA molecule, and then ligating the individual double-stranded oligonucleotides such that the desired nucleic acid sequence is obtained.

The terms “production,” “overproducting,” or “overproduction” of a protein of interest, such as an enzyme, refer herein to an increase in the production level and/or activity of said protein in a microorganism, as compared to the corresponding parent microorganism that does not comprise the modification present in the genetically modified microorganism (i.e., in the unmodified microorganism). A heterologous gene or protein can be considered to be respectively “expressed” or “overexpressed” and “produced” or “overproduced” in a genetically modified microorganism when compared with a corresponding parent microorganism in which said heterologous gene or protein is absent. In contrast, the terms “attenuating” or “attenuation” of the synthesis of a protein of interest refer to a decrease in the production level and/or activity of said protein in a microorganism, as compared to the parent microorganism. Similarly, an “attenuation” of gene expression refers to a decrease in the level of gene expression as compared to the parent microorganism. An attenuation of expression can notably be due to either the exchange of the wild-type promoter for a weaker natural or synthetic promoter or the use of an agent reducing gene expression, such as antisense RNA or interfering RNA (RNAi), and more particularly small interfering RNAs (siRNAs) or short hairpin RNAs (shRNAs). Promoter exchange may notably be achieved by the technique of homologous recombination (Datsenko & Wanner, 2000). The complete attenuation of the production level and/or activity of a protein of interest means that production and/or activity is abolished; thus, the production level of said protein is null. The complete attenuation of the production level and/or activity of a protein of interest may be due to the complete suppression of the expression of a gene. This suppression can be either an inhibition of the expression of the gene, a deletion of all or part of the promoter region necessary for expression of the gene, or a deletion of all or part of the coding region of the gene. A deleted gene can notably be replaced by a selection marker gene that facilitates the identification, isolation and purification of the modified microorganism. As a non-limiting example, suppression of gene expression may be achieved by the technique of homologous recombination, which is well-known to the person skilled in the art (Datsenko & Wanner, 2000).

Modulating the production level of one or more proteins may thus occur by altering the expression of one or more endogenous genes that encode said protein within the microorganism as described above and/or by introducing one or more heterologous genes that encode said protein(s) into the microorganism.

The term “production level” as used herein, refers to the amount (e.g., relative amount, concentration) of a protein of interest (or of the gene encoding said protein) expressed in a microorganism, which is measurable by methods well-known in the art. The level of gene expression can be measured by various known methods including Northern blotting, quantitative RT-PCR, and the like. Alternatively, the level of production of the protein coded by said gene may be measured, for example by SDS-PAGE, HPLC, LC/MS and other quantitative proteomic techniques (Bantscheff et al., 2007), or, when antibodies against said protein are available, by Western Blot-Immunoblot (Burnette, 1981), Enzyme-linked immunosorbent assay (e.g., ELISA) (Engvall and Perlman, 1971), protein immunoprecipitation, immunoelectrophoresis, and the like. The copy number of an expressed gene can be quantified, for example, by restricting chromosomal DNA followed by Southern blotting using a probe based on the gene sequence, fluorescence in situ hybridization (FISH), qPCR, and the like.

Overexpression of a given gene or overproduction of the corresponding protein may be verified by comparing the expression level of said gene or the level of synthesis of said protein in the genetically modified organism to the expression level of the same gene or the level of synthesis of the same protein, respectively, in a control microorganism that does not have the genetic modification (i.e., the parental strain or unmodified microorganism).

The microorganism genetically modified for the production of leucine and/or isoleucine provided herein comprises
a heterologous enzyme having NADP-dependent glyceraldehyde-3-phosphate dehydrogenase activity, and
an attenuation of the activity of glyceraldehyde-3-phosphate dehydrogenase A (GapA) and citrate synthase (GltA) as compared to an unmodified microorganism.

Indeed, the inventors have shown that the above genetic modifications advantageously improve leucine and isoleucine titer, productivity, and yield, as compared to a microorganism that does not comprise these modifications.

The “activity” or “function” of an enzyme designates the reaction that is catalyzed by said enzyme for converting its corresponding substrate(s) into another molecule(s) (i.e., product(s)). As is well-known in the art, the activity of an enzyme may be assessed by measuring its catalytic efficiency and/or Michaelis constant. Such an assessment is described for example in Segel, 1993, in particular on pages 44-54 and 100-112, incorporated herein by reference.

The enzyme having NADP-dependent glyceraldehyde-3-phosphate dehydrogenase activity may be either a phosphorylating or a non-phosphorylating enzyme. It is preferably GapN. GapN may be of bacterial, archaeal, or eukaryotic origin. Preferably, GapN is of bacterial origin. GapN may notably be one of those described in FIG. 4 of Iddar et al., 2005, incorporated herein by reference. In particular, the GapN enzyme may be from a species of the Streptococcus genus (e.g., from S. mutans, S. pyogenes), a species of the Bacillus genus (e.g., B. cereus, B. licheniformis, B. thuringiensis), a species of the Clostridium genus (e.g., C. acetobutylicum), or from Pisum savitum. Preferably, the GapN enzyme is from S. mutans, S. pyogenes, C. acetobutylicum, B. cereus, or P. sativum, more preferably from S. mutans. GapN preferably has at least 80%, 90%, 95%, or 100% sequence similarity or sequence identity with the GapN enzyme having the sequence of SEQ ID NO: 36, 38, 40, 42, or 44. More preferably, GapN has the sequence of SEQ ID NO: 36. GapN may be a functional variant or functional fragment of one of the GapN enzymes described herein. The corresponding gapN gene, which codes GapN, preferably has at least 80%, 90%, 95%, or 100% sequence identity with SEQ ID NO: 35, 37, 39, 41, or 43, more preferably SEQ ID NO: 35.

A “functional fragment” of an enzyme, as used herein, refers to parts of the amino acid sequence of an enzyme comprising at least all the regions essential for exhibiting the biological activity of said enzyme. These parts of sequences can be of various lengths, provided that the biological activity of the amino acid sequence of the enzyme of reference is retained by said parts. In other words, a functional fragment of an enzyme as provided herein is enzymatically active.

A “functional variant” as used herein refers to a protein that is structurally different from the amino acid sequence of a reference protein but that generally retains all the essential functional characteristics of said reference protein. A variant of a protein may be a naturally-occurring variant or a non-naturally occurring variant. Such non-naturally occurring variants of the reference protein can be made, for example, by mutagenesis techniques on the coding nucleic acids or genes, for example by random mutagenesis or site-directed mutagenesis.

Structural differences may be limited in such a way that the amino acid sequence of reference protein and the amino acid sequence of the variant may be closely similar overall, and identical in many regions. Structural differences may result from conservative or non-conservative amino acid substitutions, deletions and/or additions between the amino acid sequence of the reference protein and the variant. The only proviso is that, even if some amino acids are substituted, deleted and/or added, the biological activity of the amino acid sequence of the reference protein is retained by the variant. As a non-limiting example, such a variant of GapN conserves its NADP-dependent glyceraldehyde-3-phosphate dehydrogenase activity. The capacity of the variants to exhibit such activity can be assessed according to in vitro tests known to the person skilled in the art. It should be noted that the activity of said variants may differ in efficiency as compared to the activity of the amino acid sequences of the enzymes of reference provided herein (e.g., the genes/enzymes provided herein of a particular species of microorganism or having particular sequences as provided in the corresponding SEQ ID NO).

A “functional variant” of an enzyme as described herein includes, but is not limited to, enzymes having amino acid sequences which are at least 60% similar or identical after alignment to the amino acid sequence encoding an enzyme as provided herein. According to the present invention, such a variant preferably has at least 70%, 80%, 85%, 90%, 95%, 96%, 97%, 98%, 99%, or 100% amino acid sequence similarity or identity to the protein described herein. Said functional variant furthermore has the same enzymatic function as the enzyme provided herein. As a non-limiting example, a functional variant of GapN of SEQ ID NO: 36 has at least 70%, 80%, 85%, 90%, 95%, 96%, 97%, 98%, 99%, or 100% sequence identity to said sequence. As a non-limiting example, means of determining sequence identity are further provided below.

Preferably, the attenuation of GapA and GltA activity results from an inhibition of expression of the gapA and gltA genes as compared to an unmodified microorganism. The activity of the GapA and/or GltA enzymes may be completely attenuated. Complete attenuation is preferably due to a partial or complete deletion of the gene coding for the enzyme. Preferably, GapA has at least 80%, 90%, 95%, or 100% sequence similarity or sequence identity with the sequence of SEQ ID NO: 34. Preferably, the gapA gene has at least 80%, 90%, 95%, or 100% sequence identity with the sequence of SEQ ID NO: 33. Preferably, the gapA gene is deleted. Preferably, GltA has at least 80%, 90%, 95%, or 100% sequence similarity or sequence identity with the sequence of SEQ ID NO: 32. Preferably, the gltA gene has at least 80%, 90%, 95%, or 100% sequence identity with the sequence of SEQ ID NO: 31.

The microorganism genetically modified for the production of leucine and/or isoleucine microorganism of the present invention preferably comprises
the expression of a heterologous gapN gene coding an NADP-dependent glyceraldehyde-3-phosphate dehydrogenase, and
an attenuation of the expression of the gapA and gltA genes as compared to an unmodified microorganism.

In addition to the modifications described above, the genetically modified microorganism for production of leucine and/or isoleucine may comprise one or more additional modifications among those described below.

In particular, said microorganism may further comprise an attenuation of D-erythrose-4-phosphate dehydrogenase (GapB) activity. Preferably, production of GapB is partially or completely attenuated. Preferably, GapB has at least 80%, 90%, 95%, or 100% sequence similarity or sequence identity with the sequence of SEQ ID NO: 46. Preferably, attenuation of GapB activity results from an inhibition of the expression of the gapB gene coding said enzyme. Preferably, attenuation of expression results from a partial or complete deletion of the gapB gene. Preferably, the gapB gene has at least 80%, 90%, 95%, or 100% sequence identity with the sequence of SEQ ID NO: 45.

Said microorganism may further comprise an attenuation of glyceraldehyde-3-phosphate dehydrogenase (GapC) activity. Preferably, production of GapC is partially or completely attenuated. Preferably, GapC has at least 80%, 90%, 95%, or 100% sequence similarity or sequence identity with the sequence of SEQ ID NO: 125, 127, or 129. Preferably, said attenuation results from an inhibition of expression of the gapC gene coding said enzyme. Preferably, attenuation of expression results from a partial or complete deletion of the gapC gene. In some microorganisms, the gapC gene is a pseudogene. Thus, “gapC” as used herein may refer to a functional gene or to a pseudogene. The gapC pseudogene or functional gene, preferably has at least 80%, 90%, 95%, or 100% sequence identity with the sequence of SEQ ID NO: 47, 124, 126, or 128. In cases where gapC is a pseudogene, said pseudogene is advantageously deleted in order to avoid reversion of pseudogene into functional gene.

Preferably, the microorganism comprises an attenuation of the expression of the gapB gene and deletion of gapC pseudogene as compared to an unmodified microorganism, more preferably a deletion of the gapB and gapC genes.

The microorganism genetically modified for the production of leucine and/or isoleucine preferably further comprises increased activity of at least one of the following enzymes: acetate kinase (AckA), phosphate acetyltransferase (Pta), and acetyl-coenzyme A synthetase (Acs), as compared to an unmodified microorganism. Preferably, the microorganism comprises an overproduction of at least one protein selected from among AckA, Pta, and Acs, as compared to an unmodified microorganism. More preferably, the Pta protein is overproduction as compared to an unmodified microorganism.

Preferably, AckA has at least 80%, 90%, 95%, or 100% sequence similarity or sequence identity with the sequence of SEQ ID NO: 83. Preferably, Pta has at least 80%, 90%, 95%, or 100% sequence similarity or sequence identity with the sequence of SEQ ID NO: 61. Preferably, Acs has at least 80%, 90%, 95%, or 100% sequence similarity or sequence identity with the sequence of SEQ ID NO: 85.

Preferably, the overproduction of said one or more proteins results from an overexpression of the gene coding said protein (i.e., at least one of the genes selected from among ackA, pta, and acs). Preferably, the ackA gene has at least 80%, 90%, 95%, or 100% sequence identity with the sequence of SEQ ID NO: 82. Preferably, the pta gene has at least 80%, 90%, 95%, or 100% sequence identity with the sequence of SEQ ID NO: 60. Preferably, the acs genes has at least 80%, 90%, 95%, or 100% sequence identity with the sequence of SEQ ID NO: 84.

Preferably, the microorganism comprises an overexpression of at least one gene selected from among ackA, pta, and acs, as compared to an unmodified microorganism, more preferably an overexpression of the pta gene as compared to an unmodified microorganism.

The microorganism for the production of leucine may comprise an increased activity of at least one of the following enzymes: acetohydroxy acid synthase I (IlvBN), ketol-acid reductoisomerase (NADP(+)) (IlvC), dihydroxy-acid dehydratase (IlvD), 2-isopropylmalate synthase (LeuA*), 3-isopropylmalate dehydrogenase (LeuB), 3-isopropylmalate dehydratase (LeuCD), and branched-chain-amino-acid aminotransferase IlvE, as compared to an unmodified microorganism. Preferably, the microorganism for the production of leucine comprises an overproduction of at least one of the following proteins: IlvBN, IlvC, IlvD, LeuA*, LeuB, LeuC, LeuD, and IlvE. LeuA* is a feedback resistant (FBR) protein. The acetohydroxy acid synthase I may also be feedback resistant (IlvBN*).

The term “feedback resistant protein” as used herein refers to a protein which has been modified such that feedback inhibition of the protein (i.e., the reduction in enzyme activity mediated by the binding of the product to the enzyme) is reduced or even eliminated.

Preferably, IlvB and IlvN have at least 80%, 90%, 95%, or 100% sequence similarity or sequence identity with the sequences of SEQ ID NOs: 8 and 10, respectively. When IlvN* is overproduced rather than IlvN, said protein preferably has at least 80%, 90%, 95%, or 100% sequence similarity or sequence identity with the sequence of SEQ ID NO: 12. IlvN* comprises the substitutions G20D, V21 D and M22F when compared to SEQ ID NO: 10. Preferably, IlvC has at least 80%, 90%, 95%, or 100% sequence similarity or sequence identity with the sequence of SEQ ID NO: 20. Preferably, IlvD has at least 80%, 90%, 95%, or 100% sequence similarity or sequence identity with the sequence of SEQ ID NO: 18. Preferably, LeuA*, LeuB, LeuC, and LeuD have at least 80%, 90%, 95%, or 100% sequence similarity or sequence identity with the sequences of SEQ ID NOs: 24, 26, 28, and 30, respectively. LeuA* comprises the substitution G462D when compared to SEQ ID NO: 22. Preferably, IlvE has at least 80%, 90%, 95%, or 100% sequence similarity or sequence identity with the sequence of SEQ ID NO: 16.

Preferably, the overproduction of said one or more proteins results from an overexpression of the gene coding said protein (i.e., ilvBN (or ilvBN*), ilvC, ilvD, leuA*, leuB, leuC, and/or leuD genes). Preferably, the ilvB and ilvN genes have at least 80%, 90%, 95%, or 100% sequence identity with the sequences of SEQ ID NOs: 7 and 9, respectively. Preferably, the ilvN* gene has at least 80%, 90%, 95%, or 100% sequence identity with the sequence of SEQ ID NO: 11, wherein ilvN* codes for a protein having the substitutions G20D, V21 D, and M22F with reference to the wild-type protein having the sequence SEQ ID NO: 10. Preferably, the ilvC gene has at least 80%, 90%, 95%, or 100% sequence identity with the sequence of SEQ ID NO: 19. Preferably, the ilvD gene has at least 80%, 90%, 95%, or 100% sequence identity with the sequence of SEQ ID NO: 17. Preferably, the leuA*BCD genes have at least 80%, 90%, 95%, or 100% sequence identity with the sequences of SEQ ID NOs: 23, 25, 27, and 29, respectively, wherein the leuA* gene codes for a protein having the substitution G462D with reference to the wild-type protein having the sequence SEQ ID NO: 22. Preferably, the ilvE gene has at least 80%, 90%, 95%, or 100% sequence identity with the sequence of SEQ ID NO: 15.

Preferably, the microorganism is genetically modified for the production of leucine and comprises an overexpression of the following genes: ilvBN, ilvC, ilvD, leuA*, leuB, leuC, leuD, and ilvE, as compared to an unmodified microorganism.

Preferably, overexpression occurs by replacing the native promoter with an artificial promoter, such as the Ptrc promoter. Alternatively, a vector comprising one or more genes under the control of a strong or inducible promoter (e.g., the pCL1920 vector) may be introduced into the microorganism and the gene(s) overexpressed.

Preferably, the microorganism is genetically modified for the production of isoleucine and comprises:
the expression of a heterologous enzyme having citramalate synthase activity,
an increased activity of at least one of the following enzymes: acetolactate synthase Ill (IlvlH*), IlvC, IlvD, LeuB, LeuCD, and IlvE, as compared to an unmodified microorganism, and
attenuation of 2-isopropylmalate synthase (LeuA) activity.

IlvH* is a FBR protein.

The heterologous enzyme having citramalate synthase activity preferably has at least 80%, 90%, 95%, or 100% sequence similarity or sequence identity with the CimA enzyme having the sequence of SEQ ID NO: 75. Preferably, the heterologous enzyme having citramalate synthase activity is CimA of Methanocaldococcus jannaschii, or a functional fragment or functional variant thereof, more preferably a functional variant thereof that is feedback resistant (CimA*). Preferably, the FBR citramalate synthase has at least 80%, 90%, 95%, or 100% sequence similarity or sequence identity with the CimA enzyme having the sequence of SEQ ID NO: 77, with CimA* comprising the substitutions I47V, E114V, H126Q, T204A, L238S, and V373STOP where the sequence is not 100% identical to SEQ ID NO: 77.

The cimA gene preferably codes a CimA enzyme having at least 80%, 90%, 95%, or 100% sequence similarity or sequence identity with SEQ ID NO: 75. The cimA* gene preferably codes a CimA* enzyme having at least 80%, 90%, 95%, or 100% sequence similarity or sequence identity with SEQ ID NO: 77. Preferably, the cimA gene has at least 80%, 90%, 95%, or 100% sequence identity with SEQ ID NO: 74. Preferably, the cimA* gene has at least 80%, 90%, 95%, or 100% sequence identity with SEQ ID NO: 76, more preferably wherein the cimA* gene codes for a protein having the substitutions I47V, E114V, H126Q, T204A, L238S, and V373STOP in cases where the sequence is not 100% identical to the sequence of SEQ ID NO: 76.

Preferably, the microorganism for the production of isoleucine comprises an overproduction of at least one of the following proteins: IlvlH*, IlvC, IlvD, LeuB, LeuCD, and IlvE. Preferably, IlvH* has at least 80%, 90%, 95%, or 100% sequence similarity or sequence identity with the sequence of SEQ ID NO: 69 or 71, respectively, with IlvH* comprising the substitutions G14D and S17F compared to SEQ ID NO: 67 or the substitutions N29K and Q92STOP when compared to SEQ ID NO: 67. Preferably, IlvC has at least 80%, 90%, 95%, or 100% sequence similarity or sequence identity with the sequence of SEQ ID NO: 20. Preferably, IlvD has at least 80%, 90%, 95%, or 100% sequence similarity or sequence identity with the sequence of SEQ ID NO: 18. Preferably, LeuB, LeuC, and LeuD have at least 80%, 90%, 95%, or 100% sequence similarity or sequence identity with the sequence of SEQ ID NO: 26, 28, and 30, respectively. Preferably, IlvE has at least 80%, 90%, 95%, or 100% sequence similarity or sequence identity with the sequence of SEQ ID NO: 16.

Preferably, the overproduction of said one or more proteins results from an overexpression of the gene coding said protein (i.e., ilvIH*, ilvC, ilvD, leuC, leuD, leuB, and/or ilvE genes).

Preferably, the ilvI gene has at least 80%, 90%, 95%, or 100% sequence identity with the sequence of SEQ ID NO: 72. Preferably, the ilvH* gene has at least 80%, 90%, 95%, or 100% sequence identity with the sequence of SEQ ID NO: 68 or 70, wherein the ilvH* gene codes for a protein having the substitutions G14D and S17F or the substitutions N29K and Q92STOP in cases where the sequence is not 100% identical to the sequence of SEQ ID NO: 68 or 70, respectively. Preferably, the ilvC gene has at least 80%, 90%, 95%, or 100% sequence identity with the sequence of SEQ ID NO: 19. Preferably, the ilvD gene has at least 80%, 90%, 95%, or 100% sequence identity with the sequence of SEQ ID NO: 17. Preferably, the leuB, leuC, and leuD genes have at least 80%, 90%, 95%, or 100% sequence identity with the sequences of SEQ ID NOs: 25, 27, and 29, respectively. Preferably, the ilvE gene has at least 80%, 90%, 95%, or 100% sequence identity with the sequence of SEQ ID NO: 15. Preferably, the microorganism is genetically modified for the production of isoleucine and comprises an overexpression of the following genes: ilvIH*, ilvC, ilvD, leuC, leuD, leuB, and/or ilvE genes, as compared to an unmodified microorganism.

Preferably, LeuA has at least 80%, 90%, 95%, or 100% sequence similarity or sequence identity with the sequence of SEQ ID NO: 22. Preferably, expression of LeuA is partially or completely attenuated. Preferably, attenuation of LeuA activity results from an inhibition of expression of the leuA gene coding said enzyme. Preferably, attenuation of expression results from a partial or complete deletion of the leuA gene. Preferably, the leuA gene has at least 80%, 90%, 95%, or 100% sequence identity with the sequence of SEQ ID NO: 21.

Preferably, the microorganism is genetically modified for the production of isoleucine and comprises:
the expression of a heterologous cimA* gene,
an overexpression of the following genes: ilvIH*, ilvC, ilvD, leuC, leuD, leuB, and ilvE, as compared to an unmodified microorganism, and
an attenuation of the leuA gene.

Preferably, overexpression of an endogenous gene occurs by replacing the native promoter with an artificial promoter, such as the Ptrc promoter. Alternatively, a vector comprising one or more genes under the control of a strong or inducible promoter (e.g., the pCL1920 vector) may be introduced into the microorganism and the gene(s) overexpressed.

Preferably, one or more of any of the above FBR proteins replaces the corresponding wild-type protein in the microorganism when said protein is endogenous (e.g., IlvH* replaces wild-type IlvH in the microorganism). As a non-limiting example, the wild-type protein may be replaced with the FBR mutant by deleting the gene coding for the wild-type protein in the microorganism and incorporating the gene coding for the FBR mutant (e.g., by transforming the microorganism with a plasmid which overexpresses the gene) or by directly mutating the wild-type gene present in the microorganism such that it becomes feedback resistant.

Preferably, the microorganism is genetically modified for the production of leucine or isoleucine and further comprises:
an attenuation of the overproduction of at least one of the following proteins: soluble pyridine nucleotide transhydrogenase (UdhA), pyruvate dehydrogenase (AceEF), 2-oxoglutarate dehydrogenase (SucAB), pyruvate oxidase (PoxB), branched-chain amino acid transport system 2 carrier protein (BrnQ), branched chain amino acid/phenylalanine transport system (LivKHMGF), lactate dehydrogenase (LdhA), alcohol dehydrogenase (AdhE), methylglyoxal synthase (MgsA), fumarate reductase enzyme complex (FrdABCD), pyruvate formate lyase (PfIAB), glucose-6-phosphate 1-dehydrogenase (Zwf), phosphogluconate dehydratase (Edd), KHG/KDPG aldolase (Eda), and 6-phosphogluconate dehydrogenase (Gnd), and/or
an increase in the expression of at least one of the following proteins: NAD(P) transhydrogenase (PntAB), glutamate dehydrogenase (GdhA), leucine exporter (LeuE), and valine exporter (YgaZH),
as compared to an unmodified microorganism.

Preferably, UdhA has at least 80%, 90%, 95%, or 100% sequence similarity or sequence identity with the sequence of SEQ ID NO: 49. Preferably, AceE and AceF have at least 80%, 90%, 95%, or 100% sequence similarity or sequence identity with the sequences of SEQ ID NO: 51 and 53, respectively. Preferably, SucA and SucB have at least 80%, 90%, 95%, or 100% sequence similarity or sequence identity with the sequences of SEQ ID NO: 57 and 59, respectively. Preferably, PoxB has at least 80%, 90%, 95%, or 100% sequence similarity or sequence identity with the sequence of SEQ ID NO: 55. Preferably, BrnQ has at least 80%, 90%, 95%, or 100% sequence similarity or sequence identity with the sequence of SEQ ID NO: 87. Preferably, LivK, LivH, LivM, LivG, and LivF have at least 80%, 90%, 95%, or 100% sequence similarity or sequence identity with the sequences of SEQ ID NO: 89, 91, 93, 95, and 97, respectively. Preferably, LdhA has at least 80%, 90%, 95%, or 100% sequence similarity or sequence identity with the sequence of SEQ ID NO: 2. Preferably, AdhE has at least 80%, 90%, 95%, or 100% sequence similarity or sequence identity with the sequence of SEQ ID NO: 4. Preferably, MgsA has at least 80%, 90%, 95%, or 100% sequence similarity or sequence identity with the sequence of SEQ ID NO: 107. Preferably, FrdA, FrdB, FrdC, and FrdD have at least 80%, 90%, 95%, or 100% sequence similarity or sequence identity with the sequences of SEQ ID NO: 99, 101, 103, and 105, respectively. Preferably, PfIA and PfIB have at least 80%, 90%, 95%, or 100% sequence similarity or sequence identity with the sequences of SEQ ID NO: 109 and 111, respectively. Preferably, Zwf has at least 80%, 90%, 95%, or 100% sequence similarity or sequence identity with the sequence of SEQ ID NO: 113. Preferably Edd has at least 80%, 90%, 95%, or 100% sequence similarity or sequence identity with the sequence of SEQ ID NO: 115. Preferably Eda has at least 80%, 90%, 95%, or 100% sequence similarity or sequence identity with the sequence of SEQ ID NO: 117. Preferably Gnd has at least 80%, 90%, 95%, or 100% sequence similarity or sequence identity with the sequence of SEQ ID NO: 119.

Preferably, attenuation of expression results from a partial or complete deletion of the gene encoding said protein (i.e., at least one of the udhA, aceEF, sucAB, poxB, brnQ, livKHMGF, adhE, IdhA, frdABCD, mgsA, pflAB, zwf, edd, eda, and gnd genes).

Preferably, the udhA gene has at least 80%, 90%, 95%, or 100% sequence identity with the sequence of SEQ ID NO: 48. Preferably, the aceEF genes have at least 80%, 90%, 95%, or 100% sequence identity with the sequences of SEQ ID NOs: 50 and 52, respectively. Preferably, the sucAB genes have at least 80%, 90%, 95%, or 100% sequence identity with the sequences of SEQ ID NOs: 56 and 58, respectively. Preferably, the poxB gene has at least 80%, 90%, 95%, or 100% sequence identity with the sequence of SEQ ID NO: 54. Preferably, the brnQ gene has at least 80%, 90%, 95%, or 100% sequence identity with the sequence of SEQ ID NO: 86. Preferably, the livKHMGF genes have at least 80%, 90%, 95%, or 100% sequence identity with the sequences of SEQ ID NOs: 88, 90, 92, 94, and 96, respectively. Preferably, the IdhA gene has at least 80%, 90%, 95%, or 100% sequence identity with the sequence of SEQ ID NO: 1. Preferably, the adhE gene has at least 80%, 90%, 95%, or 100% sequence identity with the sequence of SEQ ID NO: 3. Preferably, the mgsA gene has at least 80%, 90%, 95%, or 100% sequence identity with the sequence of SEQ ID NO: 106. Preferably, the frdABCD genes have at least 80%, 90%, 95%, or 100% sequence identity with the sequences of SEQ ID NOs: 98, 100, 102, and 104, respectively. Preferably, the pflAB genes have at least 80%, 90%, 95%, or 100% sequence identity with the sequences of SEQ ID NOs: 108 and 110, respectively. Preferably, the zwf gene has at least 80%, 90%, 95%, or 100% sequence identity with the sequence of SEQ ID NO: 112. Preferably, the edd gene has at least 80%, 90%, 95%, or 100% sequence identity with the sequence of SEQ ID NO: 114. Preferably, the eda gene has at least 80%, 90%, 95%, or 100% sequence identity with the sequence of SEQ ID NO: 116. Preferably, the gnd gene has at least 80%, 90%, 95%, or 100% sequence identity with the sequence of SEQ ID NO: 118. Preferably, at least one gene selected from among udhA, aceEF, sucAB, poxB, brnQ, livKHMGF, adhE, IdhA, frdABCD, mgsA, pflAB, zwf, edd, eda, and gnd is deleted. Preferably, the genes udhA, aceEF, sucAB, and poxB are attenuated as compared to an unmodified microorganism, more preferably deleted.

Preferably, PntAB has at least 80%, 90%, 95%, or 100% sequence similarity or sequence identity with the sequences of SEQ ID NO: 121 (PntA) and 123 (PntB). Preferably, GdhA has at least 80%, 90%, 95%, or 100% sequence similarity or sequence identity with the sequence of SEQ ID NO: 63. Preferably, LeuE has at least 80%, 90%, 95%, or 100% sequence similarity or sequence identity with the sequence of SEQ ID NO: 65. Preferably, YgaZH has at least 80%, 90%, 95%, or 100% sequence similarity or sequence identity with the sequences of SEQ ID NOs: 79 (YagZ) and 81 (YagH). Preferably, the GdhA and LeuE proteins are overexpressed as compared to an unmodified microorganism.

Preferably, the overproduction of said one or more proteins results from an overexpression of the gene coding said protein (i.e., at least one of the pntAB, gdhA, leuE, and ygaZH genes). Preferably, the pntAB genes have at least 80%, 90%, 95%, or 100% sequence identity with the sequence of SEQ ID NO: 120 (pntA) and 122 (pntB). Preferably, the gdhA gene has at least 80%, 90%, 95%, or 100% sequence identity with the sequence of SEQ ID NO: 62. Preferably, the leuE gene has at least 80%, 90%, 95%, or 100% sequence identity with the sequence of SEQ ID NO: 64. Preferably, the ygaZH genes have at least 80%, 90%, 95%, or 100% sequence identity with the sequence of SEQ ID NO: 78 (ygaZ) and SEQ ID NO: 80 (ygaH).

Preferably, the gdhA and leuE genes are overexpressed as compared to an unmodified microorganism.

Preferably, the microorganism further comprises:
a) an attenuation of the expression of at least one gene selected from among udhA, aceEF, sucAB, poxB, brnQ, livKHMGF, adhE, IdhA, frdABCD, mgsA, pflAB, zwf, edd, eda, and gnd, and/or
b) an overexpression of at least one gene selected from among pntAB, gdhA, leuE, and ygaZH,
   as compared to an unmodified microorganism

According to a particularly preferred embodiment, the microorganism further comprises an attenuation of the udhA, aceEF, sucAB, and poxB genes and an overexpression of the pta, gdhA, and leuE genes.

In a further aspect, the microorganism genetically modified for the production of leucine or isoleucine as described herein is further modified to be able to use sucrose as a carbon source. Preferably, proteins involved in the import and metabolism of sucrose are overproduced. Preferably, the following proteins are overproduced:
CscB sucrose permease, CscA sucrose hydrolase, CscK fructokinase, and CscR csc-specific repressor, or
ScrA Enzyme II of the phosphoenolpyruvate-dependent phosphotransferase system and, said ScrK gene encodes ATP-dependent fructokinase, said ScrB sucrose 6-phosphate hydrolase (invertase), said ScrY sucrose porine, ScrR sucrose operon repressor.

Preferably, genes coding for said proteins are overexpressed according to one of the methods provided herein. Preferably, the microorganism overexpresses:
the heterologous cscBKAR genes of E. coli EC3132, or
the heterologous scrKYABR genes of Salmonella sp.

Genes and proteins are identified herein using the denominations of the corresponding genes in E. coli (e.g., E. coli K12 MG1655 having the Genbank accession number U00096.3) unless otherwise specified. However, in some cases use of these denominations has a more general meaning according to the invention and covers all of the corresponding genes and proteins in microorganisms. This is notably the case for the genes and proteins described herein that are not present in the microorganism (i.e., that are heterologous) such as GapN, CimA*, etc. Reference provided herein to any protein (e.g., enzyme) or gene further comprises functional fragments, mutants, and functional variants thereof. As provided herein, said functional fragments, mutants, and functional variants preferably have at least 90% similarity to said protein or gene, or alternatively, at least 80%, 90%, 95%, or even 100%, identity to said protein or gene.

A degree of sequence identity between proteins is a function of the number of identical amino acid residues or nucleotides at positions shared by the sequences of said proteins. The term “sequence identity” or “identity” as used herein in the context of two nucleotide or amino acid sequences more particularly refers to the residues in the two sequences that are identical when aligned for maximum correspondence. When percentage of sequence identity is used in reference to amino acid sequences, it is recognized that positions at which amino acids are not identical often differ by conservative amino acid substitutions, where amino acid residues are substituted for other amino acid residues having similar chemical properties (e.g., charge or hydrophobicity). When sequences differ due to conservative substitutions, percent identity between sequences may be adjusted upwards to correct for the conservative nature of the substitution. Sequences that differ by such conservative substitutions are said to have “sequence similarity” or “similarity”. Thus, the degree of sequence similarity between polypeptides is a function of the number of similar amino acid residues at positions shared by the sequences of said proteins. The means of identifying similar sequences and their percent similarity or their percent identities are well-known to those skilled in the art, and include in particular the BLAST programs, which can be used from the website http://www.ncbi.nlm.nih.gov/BLAST/ with the default parameters indicated on that website. The sequences obtained can then be exploited (e.g., aligned) using, for example, the programs CLUSTALW (http://www.ebi.ac.uk/clustalw/) or MULTALIN (http://prodes.toulouse.inra.fr/multalin/cgi-bin/multalin.pl), with the default parameters indicated on those websites.

Using the references given in GenBank for known genes, the person skilled in the art is able to determine the equivalent genes in other organisms, bacterial strains, yeasts, fungi, mammals, plants, etc. This routine work is advantageously done using consensus sequences that can be determined by carrying out sequence alignments with genes derived from other microorganisms, and designing degenerate probes to clone the corresponding gene in another organism. These routine methods of molecular biology are well-known to those skilled in the art.

Specifically, sequence similarity and sequence identity between amino acid sequences can be determined by comparing a position in each of the sequences which may be aligned for the purposes of comparison. When a position in the compared sequences is occupied by a similar amino acid or by the same amino acid then the sequences are, respectively, similar or identical at that position.

Sequence similarity may notably be expressed as the percent similarity of a given amino acid sequence to that of another amino acid sequence. This refers to the similarity between sequences on the basis of a “similarity score” that is obtained using a particular amino acid substitution matrix. Such matrices and their use in quantifying the similarity between two sequences are well-known in the art and described, for example in Dayhoff et al., 1978, and in Henikoff and Henikoff, 1992. Sequence similarity may be calculated from the alignment of two sequences, and is based on a substitution score matrix and a gap penalty function. As a non-limiting example, the similarity score is determined using the BLOSUM62 matrix, a gap existence penalty of 10, and a gap extension penalty of 0.1 or the BLOSUM62 matrix, a gap existence penalty of 11, and a gap extension penalty of 1. Preferably, no compositional adjustments are made to compensate for the amino acid compositions of the sequences being compared and no filters or masks (e.g., to mask off segments of the sequence having low compositional complexity) are applied when determining sequence similarity using web-based programs, such as BLAST. The maximum similarity score obtainable for a given amino acid sequence is that obtained when comparing a sequence with itself. The skilled person is able to determine such maximum similarity scores on the basis of the above-described parameters for any amino acid sequence. A statistically relevant similarity can furthermore be indicated by a “bit score” as described, for example, in Durbin et al., Biological Sequence Analysis, Cambridge University Press (1998).

To determine if a given amino acid sequence has at least 80% similarity with a protein provided herein, said amino acid sequence can be optimally aligned as provided above, preferably using the BLOSUM62 matrix, a gap existence penalty of 10, and a gap extension penalty of 0.1. Two sequences are “optimally aligned” when they are aligned for similarity scoring using a defined amino acid substitution matrix (e.g., BLOSUM62), gap existence penalty and gap extension penalty so as to arrive at the highest score possible for that pair of sequences. The skilled person is able to determine 80% similarity with a maximum score determined on the basis of the above-described parameters for any amino acid sequence.

Percent similarity or percent identities as referred to herein are determined after optimal alignment of the sequences to be compared, which may therefore comprise one or more insertions, deletions, truncations and/or substitutions. This percent identity may be calculated by any sequence analysis method well-known to the person skilled in the art. The percent similarity or percent identity may be determined after global alignment of the sequences to be compared of the sequences taken in their entirety over their entire length. In addition to manual comparison, it is possible to determine global alignment using the algorithm of Needleman and Wunsch (1970). Optimal alignment of sequences may preferably be conducted by the global alignment algorithm of Needleman and Wunsch (1970), by computerized implementations of this algorithm (such as CLUSTAL W) or by visual inspection.

For nucleotide sequences, the sequence comparison may be performed using any software well-known to a person skilled in the art, such as the Needle software. The parameters used may notably be the following: “Gap open” equal to 10.0, “Gap extend” equal to 0.5, and the EDNAFULL matrix (NCBI EMBOSS Version NUC4.4).

For amino acid sequences, the sequence comparison may be performed using any software well-known to a person skilled in the art, such as the Needle software. The parameters used may notably be the following: “Gap open” equal to 10, “Gap extend” equal to 0.1, and the BLOSUM62 matrix.

Preferably, the percent similarity or identity as defined herein is determined via the global alignment of sequences compared over their entire length.

As a particular example, to determine the percentage of similarity or identity between two amino acid sequences, the sequences are aligned for optimal comparison. For example, gaps can be introduced in the sequence of a first amino acid sequence for optimal alignment with the second amino acid sequence. The amino acid residues at corresponding amino acid positions are then compared. When a position in the first sequence is occupied by a different but conserved amino acid residue, the molecules are similar at that position, and accorded a particular score (e.g., as provided in a given amino acid substitution matrix, discussed previously). When a position in the first sequence is occupied by the same amino acid residue as the corresponding position in the second sequence, the molecules are identical at that position.

The percentage of identity between the two sequences is a function of the number of identical positions shared by the sequences. Hence % identity=number of identical positions/total number of overlapping positions×100.

In other words, the percentage of sequence identity is calculated by comparing two optimally aligned sequences, determining the number of positions at which the identical amino acid occurs in both sequences to yield the number of matched positions, dividing the number of matched positions by the total number of positions and multiplying the result by 100 to yield the percentage of sequence identity.

PFAM (protein family database of alignments and hidden Markov models; http://www.sanger.ac.uk/Software/Pfam/) represents a large collection of protein sequence alignments which may also be consulted by the skilled person. Each PFAM makes it possible to visualize multiple alignments, see protein domains, evaluate distribution among organisms, gain access to other databases, and visualize known protein structures.

Finally, COGs (clusters of orthologous groups of proteins; http://www.ncbi.nlm.nih.gov/COG/) may be obtained by comparing protein sequences from 43 fully sequenced genomes representing 30 major phylogenic lines. Each COG is defined from at least three lines, which permits the identification of former conserved domains.

The above definitions and preferred embodiments related to the functional fragments and functional variants of proteins apply mutatis mutandis to nucleotide sequences, such as genes, encoding said proteins.

According to a further aspect, the present invention relates to a method for the production of leucine and/or isoleucine using the microorganism described herein. Said method comprises the steps of:
a) culturing a microorganism genetically modified for the production of leucine and/or isoleucine as provided herein in an appropriate culture medium comprising a source of carbon, and
b) recovering leucine and/or isoleucine from the culture medium.

According to the invention, the terms “fermentative process,” “fermentative production,” “fermentation,” or “culture” are used interchangeably to denote the growth of microorganism. This growth is generally conducted in fermenters with an appropriate growth medium adapted to the microorganism being used.

An “appropriate culture medium” or a “culture medium” refers to a culture medium optimized for the growth of the microorganism and the synthesis of leucine or isoleucine by the cells. The culture medium (e.g., a sterile, liquid media) comprises nutrients essential or beneficial to the maintenance and/or growth of the microorganism such as carbon sources or carbon substrates, nitrogen sources; phosphorus sources, for example, monopotassium phosphate or dipotassium phosphate; trace elements (e.g., metal salts, for example magnesium salts, cobalt salts and/or manganese salts); as well as growth factors such as amino acids and vitamins. The fermentation process is generally conducted in reactors with a synthetic, particularly inorganic, culture medium of known defined composition adapted to the microorganism, e.g., E. coli. In particular, the inorganic culture medium can be of identical or similar composition to an M9 medium (Anderson, 1946), an M63 medium (Miller, 1992) or a medium such as defined by Schaefer et al. (1999). “Synthetic medium” refers to a culture medium comprising a chemically defined composition on which organisms are grown.

The term “source of carbon,” “carbon source,” or “carbon substrate” according to the present invention refers to any carbon source capable of being metabolized by a microorganism wherein the substrate contains at least one carbon atom. According to the present invention, said source of carbon is preferably at least one carbohydrate, and in some cases a mixture of at least two carbohydrates. The term “carbohydrate” refers to any carbon source capable of being metabolized by a microorganism and containing at least three carbon atoms, two atoms of hydrogen. The one or more carbohydrates may be selected from among the group consisting of: monosaccharides such as glucose, fructose, mannose, galactose, and the like, disaccharides such as sucrose, cellobiose, maltose, lactose, and the like, oligosaccharides such as raffinose, stacchyose, maltodextrins, and the like, polysaccharides such as cellulose, starch, or glycerol. Preferred carbon sources are fructose, galactose, glucose, lactose, maltose, sucrose, or any combination thereof, more preferably glucose, fructose, galactose, lactose, and/or sucrose, most preferably glucose.

The culture medium preferably comprises a nitrogen source capable of being used by the microorganism. Said source of nitrogen may be inorganic (e.g., (NH₄)₂SO₄) or organic (e.g., urea or glutamate). Preferably, said source of nitrogen is in the form of ammonium or ammoniac. Preferably, said source of nitrogen is either an ammonium salt, such as ammonium sulfate, ammonium chloride, ammonium nitrate, ammonium hydroxide and ammonium phosphate, or ammoniac gas, corn steep liquor, peptone (e.g., Bacto™ peptone), yeast extract, meat extract, malt extract, urea, or glutamate, or any combination of two or more thereof. In some cases, the nitrogen source may be derived from renewable biomass of microbial origin (such as beer yeast autolysate, waste yeast autolysate, baker's yeast, hydrolyzed waste cells, algae biomass), vegetal origin (such as cotton seed meal, soy peptone, soybean peptide, soy flour, soybean flour, soy molasses, rapeseed meal, peanut meal, wheat bran hydro lysate, rice bran and defatted rice bran, malt sprout, red lentil flour, black gram, bengal gram, green gram, bean flour, flour of pigeon pea, protamylasse) or animal origin (such as fish waste hydrolysate, fish protein hydrolysate, chicken feather; feather hydrolysate, meat and bone meal, silk worm larvae, silk fibroin powder, shrimp wastes, beef extract), or any other nitrogen containing waste. More preferably, said source of nitrogen is peptone and/or yeast extract.

According to a particularly preferred embodiment, the culture medium comprises at least one carbohydrate, such as glucose, as well as acetate and/or yeast extract and/or peptone, more preferably glucose and acetate.

The person skilled in the art is able to define the culture conditions for the microorganisms according to the invention. In particular the bacteria are fermented at a temperature between 20° C. and 55° C., preferably between 25° C. and 40° C., more preferably between about 30° C. to 39° C., even more preferably about 37° C. In cases, where a thermo-inducible promoter is comprised in the microorganism provided herein, said microorganism is preferably fermented at about 39° C.

This process can be carried out either in a batch process, in a fed-batch process or in a continuous process. It can be carried out under aerobic, micro-aerobic or anaerobic conditions, or a combination thereof (for example, aerobic conditions followed by anaerobic conditions).

“Under aerobic conditions” means that oxygen is provided to the culture by dissolving the gas into the liquid phase. This could be obtained by (1) sparging oxygen containing gas (e.g., air) into the liquid phase or (2) shaking the vessel containing the culture medium in order to transfer the oxygen contained in the head space into the liquid phase. The main advantage of the fermentation under aerobic conditions is that the presence of oxygen as an electron acceptor improves the capacity of the strain to produce more energy under the form of ATP for cellular processes. Therefore, the strain has its general metabolism improved.

Micro-aerobic conditions are defined as culture conditions wherein low percentages of oxygen (e.g., using a mixture of gas containing between 0.1 and 15% of oxygen, completed to 100% with inert gas such as nitrogen, helium or argon, etc.), is dissolved into the liquid phase.

Anaerobic conditions are defined as culture conditions wherein no oxygen is provided to the culture medium. Strictly anaerobic conditions are obtained by sparging an inert gas like nitrogen into the culture medium to remove traces of other gas. Nitrate can be used as an electron acceptor to improve ATP production by the strain and improve its metabolism.

The term “recovering” as used herein designates the process of separating or isolating the produced leucine or isoleucine using conventional laboratory techniques known to the person skilled in the art. Preferably, step b) of the method comprises a step of filtration, ion exchange, crystallization, and/or distillation, more preferably a step of crystallization. Leucine or isoleucine may by recovered from the culture medium and/or from the microorganism itself. Preferably, leucine or isoleucine is recovered from at least the culture medium.

### EXAMPLES

The present invention is further defined in the following examples. It should be understood that these examples, while indicating preferred embodiments of the invention, are given by way of illustration only. The person skilled in the art will readily understand that these examples are not limitative and that various modifications, substitutions, omissions, and changes may be made without departing from the scope of the invention.

### Methods

The protocols used in the following examples are:
Protocol 1 (Chromosomal modifications by homologous recombination, selection of recombinants and antibiotic cassette excision flanked by FRT sequences) and protocol 2 (Transduction of phage P1) used in this invention have been fully described in patent application WO2013/001055 (see in particular the “Examples Protocols” section and Examples 1 to 8, incorporated herein by reference).
Protocol 3: Construction of recombinant plasmids.

Recombinant DNA technology is well described and known to the person skilled in the art. Briefly, DNA fragments were PCR amplified using oligonucleotides (that the person skilled in the art will be able to define) and E. coli MG1655 genomic DNA or an adequate synthetically synthesized fragment was used as a matrix. The DNA fragments and chosen plasmid were digested with compatible restriction enzymes (that the person skilled in the art is able to define), then ligated and transformed into competent cells. Transformants were analysed and recombinant plasmids of interest were verified by DNA sequencing.
Protocol 4: Evaluation of L-leucine and L-isoleucine fermentation performance.

Production strains were evaluated in 500 mL Erlenmeyer baffled flasks using medium MM_LEF5 (Table 1) for leucine fermentation or MM_ILF3 (Table 2) for isoleucine fermentation adjusted to pH 6.8. A 5 mL preculture was grown at 30° C. for 16 hours in a rich medium (LB medium (10 g/L bactopeptone, 5 g/L yeast extract, 5 g/L NaCl) supplemented with 10 g/L glycerol, 1 g/L acetate, 1.5 g/L glutamate and 6 g/L succinate). It was used to inoculate a 50 mL culture to an OD₆₀₀ of 0.2. When necessary, antibiotics were added to the medium (spectinomycin at a final concentration of 50 mg·L⁻¹). The temperature of the cultures was 37° C. When the culture had reached an absorbance at 600 nm of 2 to 5 uOD/mL, extracellular amino acids were quantified by HPLC after OPA/Fmoc (Agilent Technologies) derivatization and other relevant metabolites were analysed using HPLC with refractometric detection (organic acids and glucose).

TABLE 1
Composition of MM_LEF5 medium

| Compound | Concentration (g · L⁻¹) |
|---|---|
| Citric acid•H₂O | 1.00 |
| MgSO₄•7H₂O | 1.00 |
| CaCl₂•2H₂O | 0.04 |
| CoCl₂•6H₂O | 0.0080 |
| MnSO₄•H₂O | 0.0200 |
| CuCl₂•2H₂O | 0.0020 |
| H₃BO₃ | 0.0010 |
| Na₂MoO₄•2H₂O | 0.0004 |
| ZnSO₄•7H₂O | 0.0040 |
| Na₂HPO₄ | 2.00 |
| K₂HPO₄ | 2.00 |
| (NH₄)₂HPO₄ | 8.00 |
| (NH₄)₂SO4 | 5.00 |
| NH₄Cl | 0.13 |
| FeSO₄•7H₂O | 0.01 |
| Thiamin | 0.0116 |
| MOPS | 40 |
| Glucose | 20 |
| Acetic acid | 2.5 |
| Glutamic acid | 1.5 |
| Succinic acid | 0.6 |
| Yeast Extract | 2 |

TABLE 2
Composition of MM_ILF3 medium

| Compound | Concentration (g · L⁻¹) |
|---|---|
| Citric acid•H₂O | 1.00 |
| MgSO₄•7H₂O | 1.00 |
| CaCl₂•2H₂O | 0.04 |
| CoCl₂•6H₂O | 0.0080 |
| MnSO₄•H₂O | 0.0200 |
| CuCl₂•2H₂O | 0.0020 |
| H₃BO₃ | 0.0010 |
| Na₂MoO₄•2H₂O | 0.0004 |
| ZnSO₄•7H₂O | 0.0040 |
| Na₂HPO₄ | 2.00 |
| K₂HPO₄ | 2.00 |
| (NH₄)₂HPO₄ | 8.00 |
| (NH₄)₂SO4 | 5.00 |
| NH₄Cl | 0.13 |
| FeSO₄•7H₂O | 0.01 |
| Thiamin | 0.0116 |
| MOPS | 40 |
| Glucose | 20 |
| Acetic acid | 2.5 |
| Glutamic acid | 1.5 |
| Succinic acid | 0.6 |
| Yeast Extract | 2 |
| Leucine | 0.02 |

In these cultures, leucine yield (Y_{leucine}) was expressed as followed:

$$Yleucine ( \frac{g}{g} ) = \frac{leucine produced ( g )}{glucose + acetate consumed ( g )}$$

and the isoleucine yield (Y_{isoleucine}) was expressed as followed:

$$Yisoleucine ( \frac{g}{g} ) = \frac{isoleucine produced ( g )}{glucose + acetate consumed ( g )}$$

In these cultures, leucine productivity (P_{leucine}) was expressed as followed:

$$Pleucine ( \frac{g / L}{h} ) = \frac{final leucine concentration ( g / L )}{time from inoculation ( h )}$$

and the isoleucine productivity (P_{isoleucine}) was expressed as followed:

$$Pisoleucine ( \frac{g / L}{h} ) = \frac{final isoleucine concentration ( g / L )}{time from inoculation ( h )}$$

Protocol 5: Biomass estimation.

Biomass quantity variation is monitored using a spectrophotometer (Nicolet Evolution 100 UV-Vis, THERMO®). The biomass production increases the turbidity of the growth medium. It is assayed by measuring the absorbance at a 600 nm wavelength. Each unit of absorbance corresponds to 2.2×10⁹+/−2×10⁸ cells/mL.

### Example 1: Strain Constructions

Leucine producing strains: Strains 1 to 6.

### Strain 1:

According to protocols 1, 2 and 3, strain 1 was obtained by sequentially modifying the E. coli MG1655 strain as follows:
by knocking out the lactate dehydrogenase (IdhA gene, SEQ ID NO: 1, coding LdHA of SEQ ID NO: 2), the alcohol dehydrogenase (adhE gene, SEQ ID NO: 3, coding AdhE of SEQ ID NO:4) and the methylglyoxal synthase (mgsA gene, SEQ ID NO: 5 coding MgsA of SEQ ID NO: 6),
by replacing the native promoter of the acetohydroxy acid synthase I small regulatory subunit (ilvBN genes: ilvB: SEQ ID NO: 7 coding IlvB of SEQ ID NO: 8; ilvN: SEQ ID NO: 9 coding IlvN of SEQ ID NO: 10) with an artificial Ptrc promoter (Brosius et al., 1985),
by overexpressing in the pCL1920 vector (Lerner & Inouye, 1990) the following genes organized in 2 operons under the control of the PR or PL promoter together with the cI857 allele of the thermosensitive repressor of lambda phage (SEQ ID NO: 13 coding the thermosensitive repressor protein of SEQ ID NO:14) (amplified from the pFC1 vector, Mermet-Bouvier & Chauvat, 1994):
   the ilvE gene coding the branched-chain-amino-acid aminotransferase (SEQ ID NO: 15 and 16, respectively),
   the ilvD gene coding IlvD dihydroxy-acid dehydratase (SEQ ID NO: 17 and 18, respectively),
   the ilvC gene coding IlvC ketol-acid reductoisomerase (SEQ ID NO: 19 and 20, respectively),
   the ilvBN genes coding both subunits of the acetohydroxy acid synthase I (ilvB: SEQ ID NO: 7 and 8; ilvN: SEQ ID NO: 9 and 10),
   organized into an operon under the control of the PR promoter, and
   the leuA* allele coding the leucine feedback resistant (FBR) 2-isopropylmalate synthase carrying the amino substitution G462D (IeuA: SEQ ID NO: 21 coding LeuA of SEQ ID NO 22; leuA*: SEQ ID NO: 23 coding LeuA* of SEQ ID NO: 24)
   the leuB gene coding 3-isopropylmalate dehydrogenase (SEQ ID NO: 25 and 26, respectively), and
   the leuCD genes (leuC: SEQ ID NO: 27 and leuD: SEQ ID NO: 29) coding 3-isopropylmalate dehydratase subunits (LeuC: SEQ ID NO: 28 and LeuD: SEQ ID NO: 30), organized in operon under the control of the PL promoter.

### Strain 2:

According to protocols 1 and 2, strain 2 was obtained by knocking out the citrate synthase (gltA gene, SEQ ID NO:31 coding GltA of SEQ ID NO: 32) in strain 1.

### Strain 3:

According to protocols 1, 2 and 3, strain 3 was obtained by sequentially modifying strain 1 as follows:
by knocking out the glyceraldehyde-3-phosphate dehydrogenase A (gapA gene, SEQ ID NO: 33 coding GapA of SEQ ID NO: 34),
by overexpressing the heterologous gapN gene (SEQ ID NO: 35) coding the NADP-dependent glyceraldehyde-3-phosphate dehydrogenase from Streptococcus mutans (SEQ ID NO: 36, Uniprot Q59931) by cloning it on the pCL1920 vector of strain 1, directly downstream of the PR promoter and upstream of the ilvE gene.

### Strain 4:

According to protocols 1, 2 and 3, strain 4 was obtained by sequentially modifying strain 2 as follows:
by knocking out the glyceraldehyde-3-phosphate dehydrogenase A (gapA gene, SEQ ID NO: 33 coding GapA of SEQ ID NO: 34),
by overexpressing the heterologous gapN gene (SEQ ID NO: 35) coding the NADP-dependent glyceraldehyde-3-phosphate dehydrogenase from Streptococcus mutans (SEQ ID NO: 36, Uniprot Q59931) by cloning it on the pCL1920 vector of strain 1, directly downstream of the PR promoter and upstream of the ilvE gene.

### Strain 5:

According to protocols 1 and 2, strain 5 was obtained by sequentially knocking out the gapB gene (SEQ ID NO: 45 coding GapB of SEQ ID NO: 46) coding the D-erythrose-4-phosphate dehydrogenase and the gapC pseudogene (SEQ ID NO: 47) coding the glyceraldehyde-3-phosphate dehydrogenase when this gene is intact, in strain 4.

### Strain 6:

According to protocols 1, 2 and 3, strain 6 was obtained by sequentially modifying strain 5 as follows:
by knocking out the soluble pyridine nucleotide transhydrogenase (udhA gene, SEQ ID NO:48 coding UdhA of SEQ ID NO: 49), the pyruvate dehydrogenase subunits AceE and AceF (aceEF genes, SEQ ID NOs: 50 and 52, coding AceEF of SEQ ID NOs: 51 and 53), the pyruvate oxidase (poxB gene, SEQ ID NO: 54 coding PoxB of SEQ ID NO: 55) and the 2-oxoglutarate dehydrogenase subunits SucA and SucB (sucA gene: SEQ ID NO: 56 coding SucA of SEQ ID NO: 57; sucB gene: SEQ ID NO: 58 coding SucB of SEQ ID NO: 59)
by overproducing the phosphate acetyltransferase by adding an artificial Ptrc promoter (Brosius et al., 1985) in front of the pta gene on the chromosome (pta gene, SEQ ID NO: 60 coding Pta of SEQ ID NO: 61),
by overexpressing the gdhA gene coding glutamate dehydrogenase (gdhA gene, SEQ ID NO: 62 coding GdhA of SEQ ID NO: 63) and the leuE gene coding leucine exporter (leuE gene, SEQ ID NO: 64 coding LeuE of SEQ ID NO: 65) by cloning them on the pCL1920 vector of strain 4, under the control of the PR promoter and the leuE promoter, respectively.

Isoleucine producing strains: Strains 7 to 12.

### Strain 7:

According to protocols 1, 2 and 3, strain 7 was obtained by sequentially modifying the E. coli MG1655 strain as follows:
knocking out the lactate dehydrogenase (IdhA gene, SEQ ID NO:1, coding LdhA of SEQ ID NO:2), the alcohol dehydrogenase (adhE gene, SEQ ID NO: 3, coding AdhE of SEQ ID NO:4), the methylglyoxal synthase (mgsA gene, SEQ ID NO: 5 coding MgsA of SEQ ID NO: 6), and 2-isopropylmalate synthase (leuA gene, SEQ ID NO: 21 coding LeuA of SEQ ID NO: 22)
replacing the acetolactate synthase Ill small subunit (ilvH gene, SEQ ID NO: 66 coding IlvH of SEQ ID NO: 67) with a valine and isoleucine FBR ilvH* allele coding the IlvH* protein having amino acid substitutions G14D and S17F (Park et al., 2012), and overexpressing the ilvIH*genes (ilvI gene: SEQ ID NO: 72 coding Ilvl of SEQ ID NO: 73 and ilvH* gene of SEQ ID NO: 68 coding IlvH* of SEQ ID NO: 69) by adding an artificial Ptrc promoter (Brosius et al., 1985) in front of the ilvI gene on the chromosome,
overexpressing the following genes organized in 2 operons under the control of the PR or PL promoter together with the cI857 allele of the thermosensitive repressor of lambda phage (SEQ ID NO: 13 coding the thermosensitive repressor protein of SEQ ID NO:14) (amplified from the pFC1 vector, Mermet-Bouvier & Chauvat, 1994) on the pCL1920 vector (Lerner & Inouye, 1990):
the ilvE gene coding the branched-chain-amino-acid aminotransferase (SEQ ID NO: 15 and 16, respectively),
the ilvD gene coding IlvD dihydroxy-acid dehydratase (SEQ ID NO: 17 and 18, respectively),
the ilvC gene coding IlvC ketol-acid reductoisomerase (SEQ ID NO: 19 and 20, respectively),
the ilvIH genes coding both subunits of the acetolactate synthase Ill (ilvI: SEQ ID NO: 72 coding Ilvl of SEQ ID NO: 73; ilvH: SEQ ID NO: 66 coding IlvH of SEQ ID NO: 67); more precisely, the ilvH* FBR allele coding the IlvH protein having amino acid substitutions G14D and S17F was used (ilvH* gene SEQ ID NO: 68 coding IlvH* of SEQ ID NO: 69), organized into an operon under the control of the PR promoter, and
the cimA* allele (SEQ ID NO: 76)) coding the isoleucine feedback resistant (FBR) citramalate synthase CimA* having the amino acid substitutions I47V, E114V, H126Q, T204A, L238S, V373STOP (SEQ ID NO: 77) (cimA gene coding citramalate synthase CimA originally from Methanococcus jannaschii, SEQ ID NO: 74 and 75, respectively; Uniprot: Q58787),
the leuB gene coding 3-isopropylmalate dehydrogenase (SEQ ID NO: 25 and 26, respectively), and
the leuCD genes (leuC: SEQ ID NO: 27 and leuD: SEQ ID NO: 29) coding 3-isopropylmalate dehydratase subunits (LeuC: SEQ ID NO: 28 and LeuD: SEQ ID NO: 30), organized into an operon under the control of under the PL promoter.

### Strain 8:

According to protocols 1 and 2, strain 8 was obtained by knocking out the citrate synthase (gltA gene, SEQ ID NO: 31 coding GltA of SEQ ID NO: 32) in strain 7.

### Strain 9:

According to protocols 1, 2, and 3, strain 9 was obtained by sequentially modifying strain 7 as follows:
by knocking out the glyceraldehyde-3-phosphate dehydrogenase A (gapA gene, SEQ ID NO:33 coding GapA of SEQ ID NO: 34),
by overexpressing the heterologous gapN gene (SEQ ID NO: 35) coding the NADP-dependent glyceraldehyde-3-phosphate dehydrogenase from Streptococcus mutans (SEQ ID NO: 36, Uniprot Q59931) by cloning it on the pCL1920 vector of strain 7, directly downstream of PR promoter and upstream of ilvE gene.

### Strain 10:

According to protocols 1, 2, and 3, strain 10 was obtained by sequentially modifying strain 8 as follows:
by knocking out the glyceraldehyde-3-phosphate dehydrogenase A (gapA gene, SEQ ID NO: 33 coding GapA of SEQ ID NO: 34),
by overexpressing the heterologous gapN gene (SEQ ID NO: 35) coding the NADP-dependent glyceraldehyde-3-phosphate dehydrogenase from Streptococcus mutans (SEQ ID NO: 36, Uniprot Q59931) by cloning it on the pCL1920 vector of strain 7, directly downstream of PR promoter and upstream of ilvE gene.

### Strain 11:

According to protocols 1 and 2, strain 5 was obtained by sequentially knocking out gapB gene coding the D-erythrose-4-phosphate dehydrogenase (SEQ ID NO: 45 and 46, respectively) and gapC pseudogene (SEQ ID NO: 47) coding the glyceraldehyde-3-phosphate dehydrogenase when this gene is intact, in strain 10, giving rise to strain 11 Strain 12:

According to protocols 1, 2, and 3, strain 12 was obtained by sequentially modifying strain 11 as follows:
by knocking out the soluble pyridine nucleotide transhydrogenase (udhA gene, SEQ ID NO: 48 coding UdhA of SEQ ID NO: 49), the pyruvate dehydrogenase subunits AceE and AceF (aceEF genes, SEQ ID NOs: 50 and 52, coding AceEF of SEQ ID NOs: 51and 53), the pyruvate oxidase (poxB gene, SEQ ID NO: 54 coding PoxB of SEQ ID NO: 55) and the 2-oxoglutarate dehydrogenase subunits SucA and SucB (sucA gene: SEQ ID NO: 56 coding SucA of SEQ ID NO: 57; sucB gene: SEQ ID NO: 58 coding SucB of SEQ ID NO: 59)
by overproducing the phosphate acetyltransferase by adding an artificial Ptrc promoter (Brosius et al., 1985) in front of the pta gene on the chromosome (pta gene, SEQ ID NO:60 coding Pta of SEQ ID NO: 61),
by overexpressing gdhA gene coding glutamate dehydrogenase (SEQ ID NOs: 62 and 63, respectively) and ygaZH genes coding valine exporter (ygaZ gene: SEQ ID NO: 78 coding YgaZ of SEQ ID NO: 79; ygaH gene: SEQ ID NO: 80 coding YgaH of SEQ ID NO: 81) by cloning them on the pCL1920 vector of strain 10, under the control of PR promoter and the ygaZ promoter, respectively.

### Example 2: Strain Performance

### Leucine Production:

TABLE 3
Biomass production, leucine titer, productivity and yield for
the different strains grown on the medium described in Table 1.

| | Biomass | Final leucine | | |
|---|---|---|---|---|
| | production | titer | Productivity | Y_{leucine} |
| Strain | (g/g) | (g/L) | (g/L · h) | (g/g) |
| Strain 1 | Reference | Reference | Reference | Reference |
| Strain 2 | − | + | + | + |
| Strain 3 | −− | + | ++ | + |
| Strain 4 | −− | ++ | +++ | ++ |
| Strain 5 | −− | ++ | +++ | ++ |
| Strain 6 | −− | +++ | +++ | +++ |

The symbol “+” indicates an increase of a factor up to 2, the symbol “++” an increase by a factor between 2 and 5 and “+++” an increase by a factor greater than 5, as compared to the values of reference strain 1. The symbol “−” indicates a decrease of a factor up to 2, the symbol “−−” a decrease by a factor between 2 and 4 as compared to the values of reference strain 1.

Results obtained using strain 2 (gltA deletion) show the interesting effect of inhibiting the carbon flux into both the tricarboxylic acid cycle and the biomass. An increased amount of carbon is used for leucine production and leucine yield is thus improved.

As can be seen in Table 3, the functional replacement of gapA by a gene coding an enzyme reducing NADP⁺ (gapN) leads to an improvement in strain performance. Leucine productivity is particularly increased. However, these modifications affect biomass production. This is clearly observable for strain 3.

Results obtained using strain 4 (gltA deletion and replacement of gapA by gapN) show that limitation of carbon flux into the tricarboxylic acid cycle and use of an NADPH generating enzyme advantageously improve leucine production, as leucine productivity, titer and yield are increased. Similar results were obtained with strains carrying an attenuation of the expression of the gltA gene rather than a deletion of the gltA gene, both in strain 2 and strain 4 backgrounds (data not shown).

In order to ensure the inability of strain 4 to produce NADH through the glycolysis pathway, gapB and gapC genes were deleted in strain 5. This advantageously leads to an increase in the metabolic stability.

Strain 6 (deletion of udhA, aceEF, poxB, sucAB and overexpression of pta, gdhA, leuE) exhibits a further improvement in leucine production—specifically in the final leucine titer and yield. Advantageously, the suppression of genes coding enzymes consuming NADP⁺ or leucine precursors combined with improved acetylCoA synthesis increases leucine production. In particular, the deletion of aceEF genes and the use of acetate are beneficial for leucine production.

### Isoleucine Production:

TABLE 4
Biomass production, isoleucine titer, productivity and yield, for
the different strains grown on the medium described in Table 2.

| | Biomass | Final isoleucine | | |
|---|---|---|---|---|
| | production | titer | Productivity | Y_{isoleucine} |
| Strain | (g/g) | (g/L) | (g/L · h) | (g/g) |
| Strain 7 | Reference | Reference | Reference | Reference |
| Strain 8 | − | + | + | + |
| Strain 9 | −− | + | ++ | + |
| Strain 10 | −− | ++ | +++ | ++ |
| Strain 11 | −− | ++ | +++ | ++ |
| Strain 12 | −− | +++ | +++ | +++ |

The symbol “+” indicates an increase of a factor up to 2, the symbol “++” an increase by a factor between 2 and 5, and “+++” an increase by a factor greater than 5, as compared to the values of reference strain 7. The symbol “−” indicates a decrease of a factor up to 2, the symbol “−−” a decrease by a factor between 2 and 4, as compared to the values of reference strain 7.

Results obtained using strain 8 (gltA deletion) show the interesting effect of inhibiting the carbon flux into both the tricarboxylic acid cycle and the biomass. An increased amount of carbon is used for isoleucine production and isoleucine yield is thus improved. As can be seen in Table 4, the functional replacement of gapA by a gene coding an enzyme reducing NADP⁺ (gapN) leads to improvement in strain performance. Isoleucine productivity is particularly increased. However, these modifications affect biomass production. This is clearly observable for strain 9.

Results obtained using strain 10 (gltA deletion and replacement of gapA by gapN) show that limitation of carbon flux into the tricarboxylic acid cycle and use of an NADPH generating enzyme advantageously improve isoleucine production. Indeed, isoleucine productivity, titer, and yield are increased

Similar results were obtained with strains carrying an attenuation of the expression of the gltA gene rather than a deletion of the gltA gene, both in strain 8 and strain 10 backgrounds (data not shown).

In order to ensure the inability of strain 10 to produce NADH through the glycolysis pathway, gapB and gapC genes were deleted in strain 11. This leads to an increase in the metabolic stability. Strain 12 (deletion of udhA, aceEF, poxB, sucAB and overexpression of pta, gdhA, ygaZH) exhibits a further improvement in isoleucine production. Advantageously, the final leucine titer and yield are further increased. The suppression of genes coding enzymes consuming NADP⁺ or isoleucine precursors combined with improved acetylCoA synthesis increases isoleucine production. In particular, the deletion of the aceEF genes and the use of acetate are beneficial for isoleucine production.

### REFERENCES

Anderson, (1946), Proc. Natl. Acad. Sci. USA., 32:120-128.
Bantscheff et al., (2007), Analytical and Bioanalytical Chemistry, vol. 389(4): 1017-1031.
Brosius et al, (1985), J Biol Chem, 260(6): 3539-3541
Burnette, (1981), Analytical Biochemistry, 112(2): 195-203.
Datsenko and Wanner, (2000), Proc Natl Acad Sci USA., 97: 6640-6645.
Davis & Olsen., (2011), Mol. Biol. Evol., 28(1):211-221.
Dayhoff et al. (1978), “A model of evolutionary change in proteins,” in “Atlas of Protein Sequence and Structure,” Vol. 5, Suppl. 3 (ed. M. O. Dayhoff), p.345-352. Natl. Biomed. Res. Found., Washington, D.C.
Deml et al., (2011), J. Virol., 75(22): 10991-11001.
Durbin et al., (1998), Biological Sequence Analysis, Cambridge University Press.
Engvall and Perlman (1981), Immunochemistry, 8: 871-874.
Graf et al., (2000), J. Virol., 74(22): 10/22-10826.
Henikoff and Henikoff (1992), Proc. Natl. Acad. Sci. USA, 89:10915-10919
Iddar et al. (2005), Int Microbiol., 8(4):251-8.
Lerner & Inouye, (1990), Nucleic Acids Research, 18(15): 4631
Leuchtenberger, et al, (2005) Appl. Microbiol. Biotechnol. 69, 1-8
Mermet-Bouvier & Chauvat, (1994), Current Microbiology, 28: 145-148
Miller, (1992) “A Short Course in Bacterial Genetics: A Laboratory Manual and Handbook for Escherichia coli and Related Bacteria”, Cold Spring Harbor Laboratory Press, Cold Spring Harbor, N.Y.
Needleman and Wunsch (1970), J. Mol. Biol., 48(3), 443-453.
Park et al., (2012), ACS synthetic biology, 1(11): 532-540
Park and Lee, (2010), Appl Microbiol Biotechnol 85(3):491-506
Prescott et al., (1999), “Microbiology” 4th Edition, WCB McGraw-Hill.
Sambrook and Russell, (2001), Molecular Cloning: 3rd edition, Cold Spring Harbor Laboratory Press, NY, Vol 1, 2, 3.
Schaefer et al., (1999), Anal. Biochem. 270: 88-96.
Segel (1993), Enzyme kinetics, John Wiley & Sons, pp. 44-54 and 100-112.
Yamamoto et al, (2017), Adv Biochem Eng Biotechnol. 159:103-128.

1. Microorganism genetically modified for the production of leucine and/or isoleucine, wherein said microorganism comprises the following modifications:
a) expression of a heterologous gapN gene coding an NADP-dependent glyceraldehyde-3-phosphate dehydrogenase, and
b) attenuation of the expression of gapA and gltA genes as compared to an unmodified microorganism.

2. Microorganism of claim 1, wherein the gapN gene codes an NADP-dependent glyceraldehyde-3-phosphate dehydrogenase having at least 80% identity with GapN from Streptococcus mutans.

3. Microorganism of claim 1, wherein the gapA gene is deleted.

4. Microorganism of claim 1, further comprising an attenuation of the expression of the gapB and/or gapC genes as compared to an unmodified microorganism.

5. Microorganism of claim 1, further comprising an overexpression of at least one gene selected from the group consisting of ackA, pta, and acs, as compared to an unmodified microorganism.

6. Microorganism of claim 1, wherein the microorganism is further genetically modified for the production of leucine and comprises an overexpression of the following genes: ilvBN, ilvC, ilvD, leuA*, leuB, leuC, leuD, and ilvE, as compared to an unmodified microorganism.

7. Microorganism of claim 1, wherein the microorganism is further genetically modified for the production of isoleucine and comprises:
a) the expression of a heterologous cimA* gene,
b) an overexpression of the following genes: ilvIH*, ilvC, ilvD, leuB, leuC, leuD, and ilvE, as compared to an unmodified microorganism, and
c) an attenuation of the leuA gene.

8. Microorganism of claim 1, further comprising:
c) an attenuation of the expression of at least one gene selected from among udhA, aceEF, sucAB, poxB, brnQ, livKHMGF, adhE, ldhA, frdABCD, mgsA, pflAB, zwf, edd, eda, and gnd, and/or
d) an overexpression of at least one gene selected from among pntAB, gdhA, leuE, and ygaZH,
as compared to an unmodified microorganism.

9. Microorganism of claim 8, wherein at least one gene selected from among udhA, aceEF, sucAB, poxB, brnQ, livKHMGF, adhE, ldhA, frdABCD, mgsA, pflAB, zwf, edd, eda, and gnd is deleted.

10. Microorganism of claim 1, wherein said microorganism belongs to the Escherichia genus, the Corynebacterium genus or the Streptococcus genus.

11. Method for the production of leucine and/or isoleucine comprising the steps of:
a) culturing a microorganism genetically modified for the production of leucine and/or isoleucine in an appropriate culture medium comprising a source of carbon, the microorganism genetically modified comprising expression of a heterologous gapN gene coding an NADP-dependent glyceraldehyde-3-phosphate dehydrogenase, and attenuation of the expression of gapA and gltA genes as compared to an unmodified microorganism, and
b) recovering leucine and/or isoleucine from the culture medium.

12. Method of claim 11, wherein the culture medium further comprises acetate.

13. Method of claim 11, wherein the source of carbon is glucose, fructose, galactose, lactose- and/or sucrose.

14. Method of claim 11, wherein step b) comprises a step of crystallization.

15. Microorganism of claim 4, comprising a deletion of the gapB gene and a deletion of gapC gene.

16. Microorganism of claim 10, wherein the microorganism is Escherichia coli.

17. Microorganism of claim 10, wherein the microorganism is Corynebacterium glutamicum.

18. Microorganism of claim 10, wherein the microorganism is Streptococcus thermophilus or Streptococcus salivarius.
